SEQUENCE LISTING (14 sequences)

SEQ ID NO: 1 - DNA, 14 bp, Artificial Sequence
  Synthetic construct
actgtaagtactgg

SEQ ID NO: 2 - DNA, 107 bp, Artificial Sequence
  Synthetic construct
tttctgttggtgctgatattgcattccgatctctctaaggaagtgctacacgattgaagcgctgaaccaattcgatcactcctctgaagatagagcgacaggcaagt

SEQ ID NO: 3 - DNA, 107 bp, Artificial Sequence
  Synthetic construct
tttctgttggtgctgatattgcgttccaggacactatcaactatgctacaactggctcaccacgtacacatagtactaggtcaatgaagatagagcgacaggcaagt

SEQ ID NO: 4 - DNA, 107 bp, Artificial Sequence
  Synthetic construct
tttctgttggtgctgatattgcctcctctgtatatgctgactaagaagagaaggaagtgtgtatgatcgaccgtacatgacactagaagatagagcgacaggcaagt

SEQ ID NO: 5 - DNA, 107 bp, Artificial Sequence
  Synthetic construct
tttctgttggtgctgatattgcatggtattcgtagtcgtcatacaccacggtagcacagtactatcgcgacttctccttaaggctgaagatagagcgacaggcaagt

SEQ ID NO: 6 - DNA, 107 bp, Artificial Sequence
  Synthetic construct
tttctgttggtgctgatattgcgattagtctggtcgtggaacttccagagctgactactatagtctgactaacaacattagttgagaagatagagcgacaggcaagt

SEQ ID NO: 7 - DNA, 107 bp, Artificial Sequence
  Synthetic construct
tttctgttggtgctgatattgcttggttactaatcatcacagctcggctgatcaactaaccaatcatgcgtagcatcagaccagcgaagatagagcgacaggcaagt

SEQ ID NO: 8 - DNA, 107 bp, Artificial Sequence
  Synthetic construct
tttctgttggtgctgatattgcctagctggactgattccgagactatagttctctcttgattagaggtctctcttctcgctaactgaagatagagcgacaggcaagt

SEQ ID NO: 9 - DNA, 107 bp, Artificial Sequence
  Synthetic construct
tttctgttggtgctgatattgcgaactctcgtgtgctacacataggctctcgtgtgcacacatagtatatggtatatggaactctgaagatagagcgacaggcaagt

SEQ ID NO: 10 - DNA, 107 bp, Artificial sequence
  Synthetic construct
tttctgttggtgctgatattgcagctagttcttctctgattagtcacagcgattagtgactgatatggtatgtggtgcctacgacgaagatagagcgacaggcaagt

SEQ ID NO: 11 - DNA, 107 bp, Artificial Sequence
  Synthetic construct
tttctgttggtgctgatattgcctaatcaactaatccgaccagctgtgatacacatatcgtagtggctaacgaactcttaccatagaagatagagcgacaggcaagt

SEQ ID NO: 12 - DNA, 107 bp, Artificial Sequence
  Synthetic construct
tttctgttggtgctgatattgcaggtctcactaatcttgcaataagaggactacgactatcgcatcatgatctaatcagatatcagaagatagagcgacaggcaagt

SEQ ID NO: 13 - DNA, 107 bp, Artificial Sequence
  Synthetic construct
tttctgttggtgctgatattgcgctggtctcttctcgaggagataccataaagaggactcctctcgtgtgcatgcacgctgtgatgaagatagagcgacaggcaagt

SEQ ID NO: 14 - DNA, 10 bp, Artificial sequence
  Synthetic construct
acgatagatc

(19) United States
(12) Patent Application Publication
(10) Pub. No.: US 2024/0257147 A1
(43) Pub. Date: Aug. 1, 2024

(54) SYSTEMS AND METHODS FOR IDENTIFYING A PRODUCTS IDENTITY

(71) Applicant: Nucleotrace Pty. Ltd., Victoria (AU)

(72) Inventor: Nicholas Owen, Victoria (AU)

(73) Assignee: Nucleotrace Pty. Ltd., Victoria (AU)

(21) Appl. No.: 17/266,385

(22) PCT Filed: Aug. 9, 2019
(86) PCT No.: PCT/AU2019/050835

§ 371 Date: Feb. 5, 2021

(30) Foreign Application Priority Data
Aug. 10, 2018 (AU) 2018902928
Dec. 21, 2018 (AU) 2018904900

(51) Int. Cl. G06Q 30/018; C12Q 1/6869; H03M 13/15

(52) U.S. Cl. CPC G06Q 30/018; C12Q 1/6869; H03M 13/1515

(57) ABSTRACT

This disclosure relates to verifying a product's identity. In particular. this disclosure relates to a method for verifying a product's identity. The method comprises a labelling step that comprises generating a first oligonucleotide sequence and calculating a first hash value of the first oligonucleotide sequence. The first hash value is associated with the product. The method then comprises synthesising the first oligonucleotide sequence and adding the synthesised oligonucleotide sequence to the product. Then follows a testing step. which comprises sequencing a second oligonucleotide sequence from the product. calculating a second hash value of the sequenced oligonucleotide sequence. and comparing the second hash value to the first hash value associated with the product to verify the product's identity.

### CROSS-REFERENCE TO RELATED APPLICATIONS

The present application claims priority from Australian provisional applications 2018902928 and 2018904900 the contents of which are incorporated herein by reference in their entirety.

### SEQUENCE LISTING

The instant application contains a Sequence Listing which has been filed electronically in ASCII format and is hereby incorporated by reference in its entirety. Said ASCII copy, created on Sep. 15, 2021, is named 529503_ST25.txt and is 3,867 bytes in size.

### TECHNICAL FIELD

This disclosure relates to verifying a product's identity. For example, but not limited to, this disclosure relates to verifying that a product's identity within a supply chain.

### BACKGROUND

Counterfeiting and piracy has increased substantially over the last two decades, with counterfeit and pirated products found in almost every country across the globe and in virtually all sectors of the economy. Estimates of the levels of counterfeiting and the value of such products vary. However, the value of global trade in counterfeit and pirated products in 2013 was estimated at $461 billion (OECD and EUIPO, 2016, Trade in Counterfeit and Pirated Goods: Mapping the Economic Impact). For example, counterfeit drugs are responsible for one million deaths and cost the industry $100 billion each year. Recent studies estimate that 10% of drugs sold each year are counterfeit, a number that is anticipated to increase with the rise of online pharmacies and 3D-printed medicines. The rapidly expanding medicinal and recreational cannabis markets are also particularly exposed to counterfeiters who may produce compositionally similar but substandard products with basic equipment.

Product serialisation and next generation blockchain-based supply chain monitoring technologies have attempted to address this threat. However, unlike crypto currencies, the blockchain is only a proxy for whatever physical goods change hands in a supply chain. Fundamentally, these ‘next generation’ solutions still rely on insecure package technologies such as inks, dyes, barcode, QR codes, RFIDs, holograms, and/or IoT devices. Existing package technologies additionally only permit traceability from the point of finished product manufacture to the point when an item is unpackaged. The capacity to trace all ingredients upstream from the point of finished product manufacture as well as downstream from the point where a product is unpackaged remains a significant challenge. Downstream tracing and identification is particularly important in circumstances where products are sold unpackaged, or two or more products are recombined and repackaged to form a third product. This capability is also permits all ingredients in a product that is suspected to be sub-standard to be rapidly traced back to their origin.

The disclosed invention described herein is a system for product tracing and verification where supply chain information is stored in physical oligonucleotide tags that are integrated into a product and backed up on an immutable blockchain. Core capabilities of the disclosed invention include full unbroken supply chain coverage, high resolution tracing (at the level of an ingredient and product unit), automatic transfer of chain information upon product mixing (no requirement to authenticate each transaction), last legitimate node traceback capabilities, protection against counterfeiting, and product authentication.

Applications include but are not limited to: certified products (sustainable, fair trade, Kosher and Halal), palm oil, pharmaceuticals, cannabis (plant to product tracing), misused products (ie. products that may be used as illicit drug precursors), milk products and infant milk formula, wine, cosmetics, precious stones, chemicals, fertilizers, bank notes, casino chips, luxury items and ammunition.

### SUMMARY

A method for verifying a product's identity comprises:
generating a first oligonucleotide sequence:
calculating a first hash value of the first oligonucleotide sequence, the first hash value being associated with the product:
synthesising the first oligonucleotide sequence:
adding the synthesised oligonucleotide sequence to the product:
sequencing a second oligonucleotide sequence from the product:
calculating a second hash value of the sequenced oligonucleotide sequence: and
comparing the second hash value to the first hash value associated with the product to verify the product's identity.

The labels “first” and “second” do not necessarily denote an order in a supply chain, so that, for example, the first hash value is not necessarily the hash value at the very beginning of the supply chain but can be anywhere within the chain. In this sense, the first hash value may also be referred to as original, new or generated hash value. Similarly, the second hash value may be referred to as sampled, sample or test hash value.

The first hash value may be incorporated onto a package containing the product, as a hash value, barcode of the hash value, QR code or the hash value or other identifier associated with the hash value.

The first hash value may be stored in a block chain. The block chain may be part of a public, distributed ledger.

Calculating the first hash value and the second hash value may be based on additional data and the additional data may comprise one or more of:
product identifier;
entity identifier;
shared secret;
padding data;
public key;
time stamp;
counter; and
product-unique product identifier.

The method may further comprise generating the first oligonucleotide sequence by encoding a digital word into the oligonucleotide sequence.

Encoding the digital word may be based on an error-correcting code and may comprises:
generating Hamming code words;
mapping sets of the Hamming code words to a Galois field, and
generating a Reed Solomon (RS) code word to thereby generate a robust code word that is robust against sequencing and synthesis errors.

The digital code word may be private to an entity performing the method.

Calculating the first hash value may comprise storing the first hash value on a database, and comparing the second hash value to the first hash value may comprise retrieving the first hash value from the database.

The method may further comprise amplifying the second oligonucleotide sequence by a polymerase chain reaction (PCR) using a secret set of primers which hybridise to primer sites on the second oligonucleotide sequence.

An entity downstream in a supply chain may add a third oligonucleotide sequence to the product.

Adding the third oligonucleotide sequence to the product may comprise calculating a third hash value associated with the product. The third oligonucleotide sequence may be another/second original, new or generated hash value.

The third hash value may be calculated based on one or more upstream hash values.

The third hash value may be calculated based on the one or more upstream hash values to thereby represent an order of added oligonucleotide sequences forming a chain of hash values.

The method may further comprise:
sequencing the third oligonucleotide sequence:
calculating a fourth hash value for each of multiple combinations of the second hash value and the fourth hash value: and
comparing the fourth hash value for each of the multiple combinations to the third hash value to identify the product's identity where one of the multiple combinations provides a match.

The fourth hash value may be another/second sample, sampled or test hash value.

The method may comprise identifying an upstream node for which the fourth hash value for one of the multiple combinations matches and calculating hash values only for combinations that relate to nodes downstream from the identified upstream node.

Adding the third oligonucleotide sequence to the product may comprise facilitating ligation of the third oligonucleotide sequence to the first oligonucleotide sequence.

The third oligonucleotide sequence added by the entity downstream in the supply chain may be indicative of a position of the entity within the supply chain.

Sequencing the second oligonucleotide sequence may comprise amplifying the oligonucleotide from the product using locked nucleic acids (LNA) primers.

Calculating the second hash value may comprise decoding the sequenced oligonucleotide sequence in one direction and upon unsuccessful decoding, decoding the sequenced oligonucleotide sequence in an opposite direction.

The method may further comprise aligning the sequenced second oligonucleotide sequence against a stored oligonucleotide sequence, wherein calculating the second hash value is based on the aligned nucleotide sequence.

Generating the first oligonucleotide sequence may be based on multiple code symbols and the method may comprise aligning the sequenced second oligonucleotide sequence against the multiple code symbols.

Generating the first oligonucleotide sequence may comprise generating multiple codewords and the method may comprise aligning the sequenced second oligonucleotide sequence against previously decoded codewords or a database of codewords.

The method may further comprise determining a sequencing error and selectively, based on the sequencing error, performing alignment against multiple code symbols or against multiple codewords.

A method for manufacturing an identifiable product comprises:
manufacturing the product:
generating a first oligonucleotide sequence:
calculating a first hash value of the first oligonucleotide sequence, the first hash value being associated with the product:
synthesising the first oligonucleotide sequence: and
adding the synthesised oligonucleotide sequence to the product to allow sequencing and comparing a second hash value of the sequencing result to the first hash value to verify the product's identity.

A method of verifying a product's identity comprises:
providing a product to which a first oligonucleotide has been added,
obtaining the sequence of the first oligonucleotide and calculating a hash value from the sequence, and
comparing the hash value to a predetermined value for the product to verify the product's identity.

Software, when executed by a computer, causes the computer to perform the above method.

An identifiable product comprises:
one or more product constituents: and
a synthesised oligonucleotide sequence added to the one or more product constituents, wherein the synthesised oligonucleotide sequence is associated with a first hash value to allow comparing a second hash value of a result from sequencing the synthesised oligonucleotide sequence to the first hash value to verify the product's identity.

The product may further comprise a package containing the product, wherein the first hash value is incorporated onto the package.

Optional features provided for one of the aspects above equally apply as optional features to other aspects including method, software and product aspects.

### BRIEF DESCRIPTION OF DRAWINGS

An example will now be described with reference to the following drawings:

FIG. 1 illustrates a method for verifying a product's identity.

FIG. 2 illustrates a system for verifying a product's identity.

FIG. 3a illustrates a computer system and key information exchanges for the blockchain-oligonucleotide tag approach disclosed.

FIG. 3b illustrates a computer system and key information exchanges in a second variation of the blockchain-oligonucleotide tag approach disclosed.

FIG. 4 illustrates a computer system for product sampling and the use of error detecting and correcting code to compute a hash of a DNA codeword, H(C_{DNA}), to validate a product.

FIG. 5 illustrates Oligonucleotide Tag Methodology 1 (OTM1) where product or node information is stored in oligonucleotide fragments, and node order is stored remotely.

FIG. 6 illustrates how information may be added to a product with OTM1.

FIG. 7 illustrates one way that supply chain information stored in physical oligonucleotides with OTM1 can be linked to a database or distributed ledger.

FIG. 8 illustrates Oligonucleotide Tag Methodology 2 (OTM2) where oligonucleotide fragments contain both product or product information and node placement/order information.

FIG. 9 illustrates how information may be added to, and recovered from, a product labelled using OTM2.

FIG. 10 illustrates one way that supply chain information stored in physical oligonucleotides with OTM2 can be linked to a database or distributed ledger.

FIG. 11 illustrates Oligonucleotide Tag Methodology 3 (OTM3) where oligonucleotide fragments contain node or product information and are sequentially ligated together to record the order added.

FIG. 12 illustrates how information may be added to, and recovered from, a product using OTM3.

FIG. 13 illustrates one way that supply chain information stored in physical oligonucleotides with OTM3 can be linked to a database or distributed ledger.

FIG. 14 illustrates the processes and inputs for computing hash functions at and between nodes in a hash chain, list or tree.

FIG. 15 illustrates different methodologies for computing genesis hash values with a DNA codeword (C_{DNA}) input.

FIG. 16 illustrates a product labelled with OTM1 methodology and how information contained in the oligonucleotide may be stored using a binary hash tree approach or simple hash list.

FIG. 17 illustrates a product labelled with OTM1 undergoing a fork (i.e. product is split) where data is stored using binary hash tree methodology.

FIG. 18 illustrates an example of two products labelled with OTM1 undergoing a merge (i.e. product is mixed) and data is stored using binary hash tree methodology.

FIG. 19 illustrates an expanded example of two products labelled with OTM1 undergoing a merge and then a fork where data is stored using binary hash tree methodology.

FIG. 20 illustrates an example of a product labelled with OTM1, where a node incorporates alternative information and no oligonucleotide tag added.

FIG. 21 illustrates an expanded example of two products labelled with OTM1 undergoing a merge and then a fork where data is stored using binary hash tree methodology and seven nodes do not incorporate oligonucleotide tag information, H(C_{DNA}).

FIG. 22 illustrates a collapsed version of FIG. 21 that only includes nodes where pC_{DNA} is added, where FIG. 21 must be recreated from a product sample that only contains information in FIG. 21.

FIG. 23 illustrates how pC_{DNA} in a product may be cryptographically linked to a package identifier technology.

FIG. 24 illustrates how a package identifier technology may be updated as new pC_{DNA} are added to a product.

FIG. 25 illustrates an example of two products labelled with OTM1 undergoing a merge to form a final product, where the hash value at the merge point is used as a unique identifier on a package, where the package identifier is used to update chain information are nodes where no pC_{DNA} is added, and where the hash chain or tree is recovered and restored from pC_{DNA} in an unpackaged product.

FIG. 26 illustrates a public key encryption protocol used to transfer information between two parties, where the transaction may be recorded on a distributed ledger and protected by blockchain.

FIG. 27 illustrates a system where oligonucleotide tag information is transferred between digital wallets, stored on a distributed ledger, and protected by blockchain.

FIG. 28 illustrates key information transfers between one or more oligonucleotide labelled products that are mixed, unpackaged, split, and repackaged.

FIG. 29 illustrates the process of product sampling and oligonucleotide tag sequencing.

FIG. 30a illustrates a methodology for encoding Hamming symbols Ham(n, k) in Z₄ using the set of nucleotides {A, C, G, T}.

FIG. 30b illustrates how a set of Hamming DNA symbols may be mapped to elements in a Galois Field (GF).

FIG. 30c illustrates how a Reed Solomon (RS) DNA codeword is assembled from Hamming symbols mapped to a Galois Field (GF).

FIG. 31 illustrates a methodology of decoding Reed Solomon (RS) DNA codewords.

FIG. 32 illustrates an example of how a codeword is encoded into an oligonucleotide, encrypted, manufactured, added to a product, sampled from a product, decoded, and validated against a database.

FIG. 33 illustrates the steps to encode RS[9,5] DNA codeword. The encoding steps shown in this diagram include the construction of (A) Ham[7,4] encoded blocks to form the DNA library of size S_{DNA}=128 symbols (S_{DNA}=Sₛ throughout) , which were (B) mapped to symbols in the finite Galois Field GF(2⁷) =GF(128). These symbols were used to (C) assemble RS[9,5] codewords from S_{DNA} according to established Reed-Solomon encoding methodology.

FIG. 34 illustrates nanopore DNA sequencing error data.

FIGS. 35 and 36 illustrate decoding steps.

FIG. 37 illustrates an analysis of decoding time against database size.

FIG. 38 illustrates decoding time versus sample size for Steps A-C.

### DESCRIPTION OF EMBODIMENTS

This disclosure constraints of existing supply chain monitoring technologies by ‘seeding’ a blockchain with a product-integrated synthetic oligonucleotide (“oligo” herein) marker encoded with a unique identifier. In this approach, a marker/s is added to each individual item (i.e. products or product ingredients) that contain information about a product and/or a product's supply chain. The oligonucleotide tag/s in a product may be cryptographically linked to other package technologies (inks, dyes, holograms, barcodes, QR codes, RFID, silicon dioxide encoded particles, IoT devices, etc.) at a point downstream in a supply chain to permit functionalities such as temperature tracking, geo-tracking, real-time tracking, or barcode scanning. The disclosed approach may be integrated into blockchain architecture to automate and secure information transfers.

It is noted that some steps described herein are steps that are preferably implemented within a computer environment. In that sense, there are provided computer systems with respective processors and program memory to store software code that causes the processor/computer to perform the described steps. The program memory may be a non-transitory computer-readable medium with the software code stored thereon. In one example, there is one computer system for the initial manufacturer (genesis), one computer system for each intermediate entity, which may be further manufactures or quality assurance entities, and one computer system for the final recipient of the product. The computer-implemented steps may be implemented on a distributed computing platform (“cloud”) such as Amazon AWS or others. When reference is made to “secret” data, such as keys, words or sequences, this is to mean that only a select user or number of users are able to access such data, such as by their read access to the respective digital storage location (file, folder, web-drive, etc.) or by their personal decryption key provided through a smart card or a passphrase provided from the users' own recollection that de-crypts the secret data. The secret data is not accessible by/protected from other users.

The approach disclosed here addresses five important considerations of supply chain monitoring:
a) Security: a product-integrated approach is needed to link a product to a distributed database (blockchain), decentralised or centralised database:
b) Coverage: an oligo-trace allows supply chain information to be recorded on a blockchain or centralised database at the point where ingredients are manufactured and additionally traced downstream from the point where a product is unpackaged. This permits unbroken coverage of an entire supply chain.
c) Information transfer: Information encoded into an oligonucleotide tag that is added to a product is transferred ‘automatically’ when a product is mixed (merged) or split (forked). This ingredient traceability in recombined products and unpackaged products.
d) Traceback capability: Traceback capability permits the identification of leaking and fraudulent nodes in a supply chain from a recombined end product alone. This capability is useful for tracing products that are sold into unauthorised markets, stolen, diluted or cut, or misused (e.g. products that may be used as precursors for illicit drugs).
e) Chain repair: Unique identifier information may be recovered from oligo-tagged products where package technologies have been either removed or damaged. This allows broken chains/trees to be repaired. A secondary product may be repackaged with package identifier technologies encoded with information derived from oligo-tags in a product.

In one example Oxford Nanopore DNA sequencing technology is used. Oxford Nanopore is a DNA sequencer that offers portability and low read latency, which permits real-time sample recovery and decoding in the field. In a further example the DNA tag sequence and associated information is stored on a distributed ledger or blockchain, such as Bitcoin, Ethereum or an independent blockchain. Each time the product is tested or transferred, the distributed ledger employs a consensus mechanism to update the ledger in light of the transfer of the product. This creates a secure chain-of-custody log for a particular item or ingredient.

It is noted that the term ‘blockchain’ is used broadly herein to denote a “hash of hashes”. In this sense, the blockchain does not necessarily have to be public, distributed and based on a proof of work or stake, but may be stored on a trusted database that can be authenticated using existing technologies, such as SSL certificates issued by Verisign Inc., for example. Each block in such a blockchain comprises a hash value that is calculated from all the previous blocks leading to the advantage that it becomes practically impossible to tamper with the earlier blocks. Further, the chain of blocks can be verified without disclosing the actual data within the blocks by publishing only the hash values. This will be described in further detail below.

Nucleic acid molecules are used herein as molecular tags (also referred to as “taggants”). It is an advantage that these molecular tags are inherently stable, information dense, non-toxic, and synthesised and sequenced using commercially mature technologies (such as chain termination sequencing, sequencing by synthesis, nanopore sequencing, single molecule real-time sequencing, and combinatorial probe anchor sequencing technologies, for example.) Non-biological information may be encoded in fragments of DNA or RNA using the nucleic acid base (b) ‘alphabet’, where the set of letters available is S={A (adenine), C (cytosine), G (guanine), T (thymine)} for DNA and {A (adenine), C (cytosine), G (guanine), U (uracil)} for RNA, where the size of the set is s=4. This base-four system allows vast amounts of information to be stored in relatively short fragments of DNA, with the number of unique taggant codewords available for a string length n letters being wₙ=sⁿ. This means, a digital code word can be encoded into the nucleotide sequence in the sense that a binary representation of data can be mapped to the quaternary DNA alphabet and encoded into the sequence. The binary code word can be any piece of data that is ordinarily stored on computer memory.

While most examples provided herein relate to the use of four-letters, it is equally possible to use oligonucleotide sequences with less, such as only two letters in a binary way, or more than the four listed above. Additionally, it is also possible to use a five letter system comprised of {A, C, G, T, U}.

The amount of information that can be encoded into an oligonucleotide codeword is defined by the size the oligonucleotide fragment and the arrangement of nucleotides, or subsets of nucleotides, as representative of a binary, ternary, quaternary, . . . , n-ary code. The total set of possible unique codes (codeword space) for each primer pair is essentially limitless for practical purposes for oligonucleotide fragments>100 b. In some instances, direct encoding, where one nucleotide is mapped to one symbol in an alphabet of four letters, may not be feasible because of sequencing and synthesis errors. Therefore, redundancy and error detecting and correcting capability may be incorporated into taggant design to increase decoding reliability. Illustrative examples of encoding systems that have built in redundancy and/or error detecting and correcting capabilities include Hamming, Reed-Solomon and Fountain encoding, for example noting that other error-correcting codes can be used.

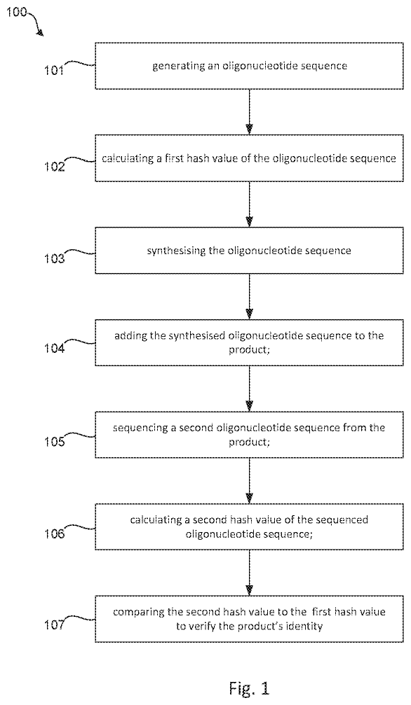

FIG. 1 illustrates a method 100 for verifying a product's identity in the sense of verifying that the product originates from the correct manufacturer. The method first comprises generating 101 an oligonucleotide sequence comprised of the four letters A, T, G and C for a DNA sequence or A, U, G and C for an RNA sequence. The oligonucleotide sequence may be represented as a string or a binary vector with 00 representing ‘A’, 01 representing ‘C’, 10 representing ‘G’ and 11 representing ‘T’. Oligonucleotide sequences may be arranged into sets of strings that represent ASCII symbols. An ASCII codeword may then be assembled from the set of ASCII symbols.

Other representations of the sequence may equally be possible and this applies throughout this disclosure where reference is made to a oligonucleotide sequence. In other words, the term oligonucleotide sequence can have multiple forms, including digital forms of data representing the sequence or chemical forms comprising the actual molecule that includes the chemical bases. If this distinction is not clear from the context, it is clarified by the terms “digital form” and “chemical form”. Throughout this document the following symbols are also used to clarify the context: (i) Cₓ, is an ASCII codeword, (ii) C_{DNA} is an oligonucleotide codeword, (iii) pC_{DNA} is the physical or chemical form of an oligonucleotide tag, and (iv) H(C_{DNA}) is a hash of a DNA codeword C_{DNA}.

In one example, the step of generating 101 the digital form of the sequence comprises an encoding step where a digital value is encoded into the sequence. The digital value may be a product code or manufacturing code or simply a random number that is not associated with any particular identifying functionality. The encoding step will be described in more detail below and essentially ensures that the sequence can meet biological constraints and can be recovered in a way that is robust against sequencing errors.

Method 100 continues by calculating 102 a first hash value of the oligonucleotide sequence. The hash value is calculated by a hash function which can take a range of different forms depending on the security requirements of the overall system. For example, a hash value may be calculated by multiplicative hashing where the overall number of different sequences is limited and therefore collision is unlikely. In other examples, more sophisticated functions, such as MD5 or preferably, SHA-2 or SHA-3 can be used. Since these sophisticated functions are highly optimised, the computational burden is minimal and therefore, there is little downside to using a hash function that is more sophisticated than required by this particular application.

After, before, or during calculating the hash value, the oligonucleotide sequence is synthesised 103 using known techniques and added 104 to the product.

This may involve mixing the synthesised (chemical form) of the sequence into the product. The product may then pass through a supply chain to reach a recipient, such as the end customer or an intermediate manufacturer or quality control agent.

It is now desired that the recipient can verify the identity of the product. Therefore, the recipient sequences 105 a second oligonucleotide sequence from the product, where it is unknown whether that sequence is the same as the sequence added by the original (or ‘upstream’) manufacturer. To verify this, the intermediary can calculate 106 a second hash value of the sequenced oligonucleotide sequence and compare 107 the second hash value to the first hash value to verify the product's identity. If the second hash value is identical to the first hash value, the product's identity is verified. If the hashes are different, the product's identity is not verified.

The hash value may also be calculated based on additional data that may be a product identifier, entity identifier of the handling entity at that point, shared secret, public key, time stamp, counter, or product-unique product identifier that is unique to that particular individual “instance” of the product. This additional data may either be concatenated with the oligonucleotide sequence before the hash is calculated or the hash of the oligonucleotide sequence may be concatenated with the additional information and another hash calculated on the result. The important aspect is that any minor chance in the additional data leads to a completely different hash and it is practically impossible to change the additional data such that the hash stays the same or to determine the additional data from the hash alone.

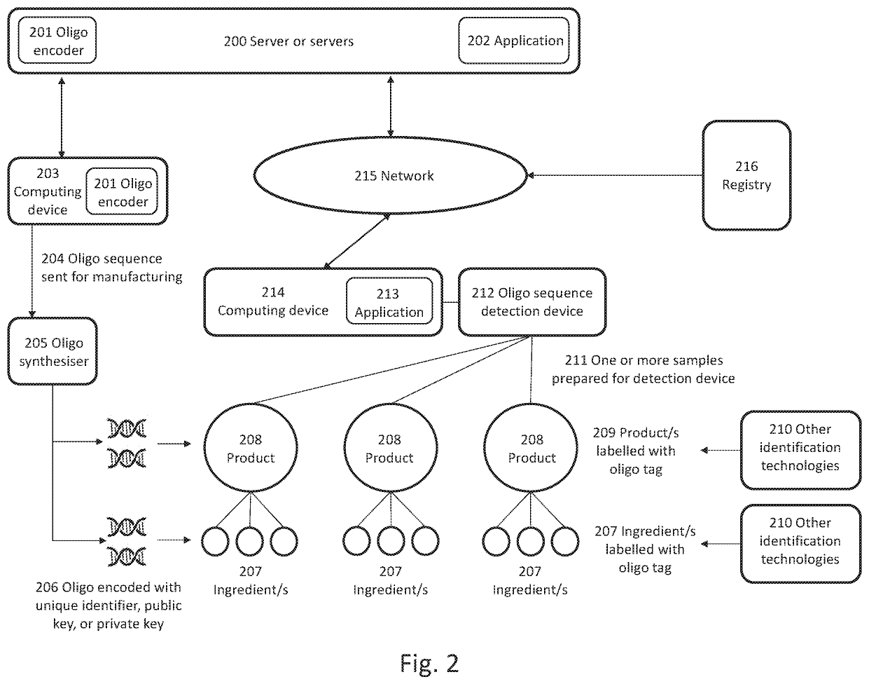

FIG. 2 is a system 200 for verifying the product's identity. The system comprises an oligonucleotide encoder 201 that may be implemented on a remote server or set of servers 202, or on a local computing device 203 . The oligonucleotide code is sent 204 for manufacture to a machine 205 that synthesizers oligonucleotides. One or more different encoded oligonucleotide fragments are then incorporated 206 into the ingredients 207 or end product 208. At a packaging step 209 other package identification (PI) technologies 210 may be included such as barcodes, QR codes, RFID, inks, dyes, encoded silicon dioxide particles, IoT devices, etc. With respect to sampling, one or more samples may be prepared and barcoded, pooled together 211, and sequenced on a sequencing device 212. The sequenced data is transmitted to a decoding application 213 either on a local computing device 214 or over a network 215 connected to the one or more remote servers 200, where it is decoded, optionally hashed, and compared to a local or distributed registry 216 of associated data, such as hashes calculated of the oligonucleotide sequence created by encoder 201.

The following description provides Information transfers and key components of an augmented oligo label—distributed ledger approach.

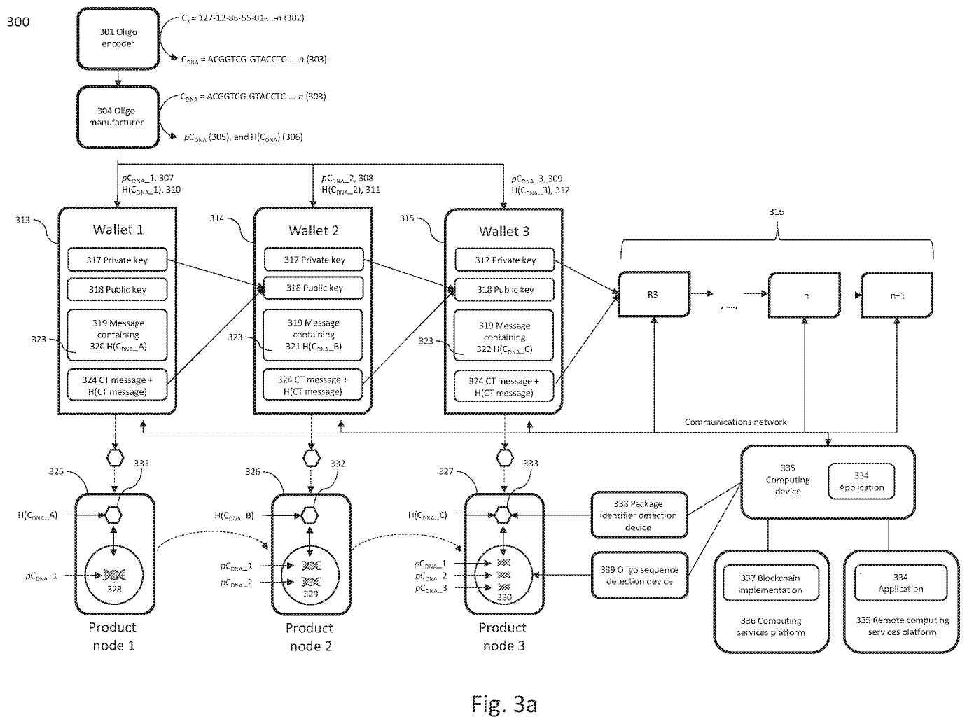

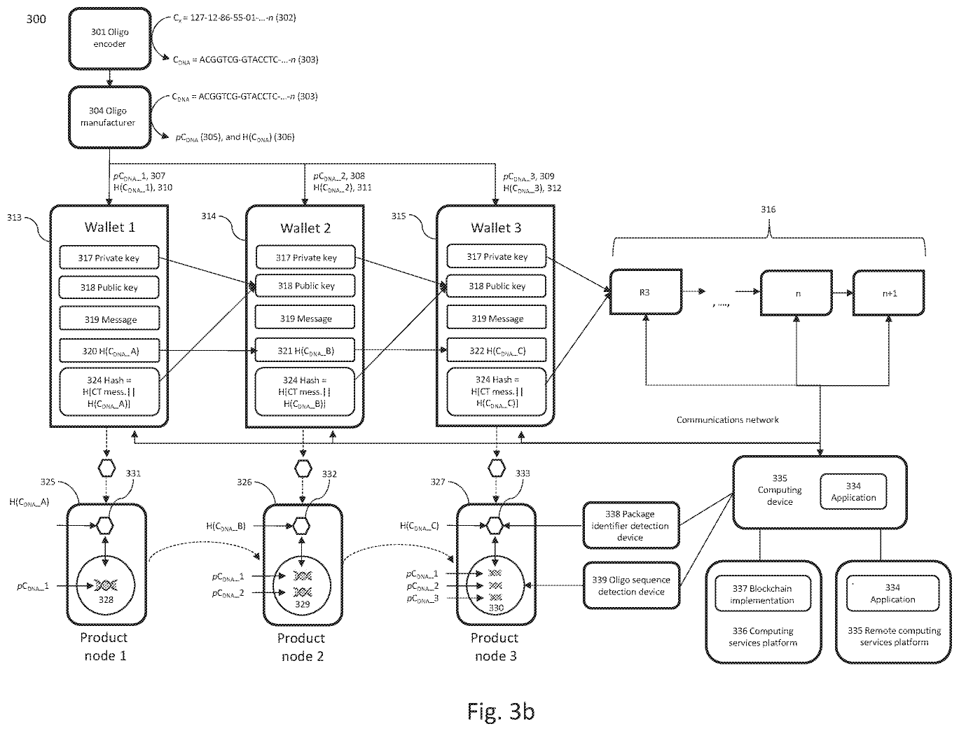

FIG. 3a illustrates a computer system 300 for information exchanges using the blockchain—oligo approach disclosed herein. System 300 comprises an oligo encoder module 301 to encode a codeword of ASCII symbols 302 (Cₓ) into a Z₄ oligonucleotide sequence 303 (C_{DNA}), where for e.g. {A, C, G, T}→{0, 1, 2, 3}→{00, 01, 10, 11}. The oligonucleotide sequence 303 is sent to a oligonucleotide manufacturer 304, where the physical oligonucleotide sequence 305 (i.e. chemical form, pC_{DNA}) with base pairs {A, C, G, T, and possibly U} is manufactured. The physical oligonucleotide sequence 305 and a hash of C_{DNA}, H(C_{DNA}) 306, is sent 307, 308, 309, 310, 311, 312 to and authorized product manufacturer 313, secondary product manufacturer 314, or other members 315, 316. One or optionally more pC_{DNA} encoded with different codewords and/or unique identifier sequences 307, 308, 309 together with a hash of these encoded fragments H(C_{DNA}) 310, 311, 312 may be sent to these members which are represented by the digital wallets 313, 314, 315 and 316.

The process by which a chain or tree is created is shown in the manufacturers wallet 313. In Wallet 1 313 the manufacturer uses a private key 317 and public key 318 to create a genesis hash and/or genesis signature of the transaction to start the chain of identity. The public key can be applied to the genesis signature to verify the manufacturer. The manufacturer's wallets also include a message 319 that may include information such as the batch number, expiry date, manufacturing facility, quality control data, or other. The message 319 in the form shown in FIG. 3a also includes a node hash 320, 321, 322 that contains H(C_{DNA}) 310, 311 and 312, respectively.

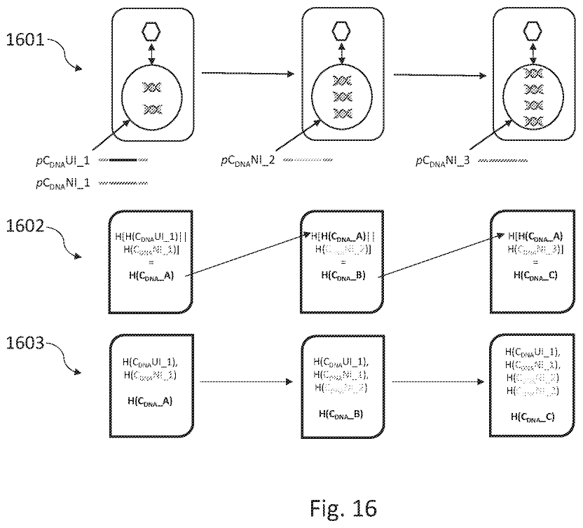

Methodologies for computing hash values 320, 321 and 322 in wallets 313, 314 and 315, respectively, are disclosed in detail below and in FIGS. 16-22. Briefly, the first hash 320 may either be H(C_{DNA}) or a hash of one or more H(C_{DNA}) optionally concatenated to zero or more of X or a hash of X, where X={a second H(C_{DNA}), time stamp, counter, alternative identifier, random number or padding text}. The second hash 321 is a hash of 320 concatenated to one or more of X or a hash of X. The third hash 322 is a hash of 321 concatenated to one or more of X or a hash of X, and so on. If a binary hashing is taken, the hash at a particular node includes a cumulative hash value computed at previous nodes plus some new information added at the node. This structure is analogous to a blockchain and presents a number of key advantages, which are disclosed in detail below.

Information in 323 may also include a message 319. A message may include information such as the product batch number, expiry date, manufacturer, manufacturing facility, timestamp, custody information, or quality control and analysis information, for example. To make a transfer, the information in 323 is encrypted into ciphertext (CT). The CT and a hash of the CT 324 is signed with the sender's private key 317 and sent to the receiver using the receiver's public key 318. A hash of the cipher text is included to ensure information in 323 has not been tampered with. Additional products may be mixed and their hash trees merged, or split and their hash trees forked, in a similar way. Note that the pC_{DNA} in products is automatically transferred to the recombined product upon mixing or splitting. The information transfer processes described here apply to all wallets 313, 314, 315, 316.

As the product is transferred between nodes in a supply chain and new pC_{DNA} are optionally added, the product may be repackaged 325, 326, 327. The addition of pC_{DNA} to a product to mark a particular event, or due to the mixing of a second tagged product, is shown in 328, 329, and 330. The information contained in a product may optionally be encrypted and displayed with a package identifier technology using the node hash value level at the point of packaging, or another node hash value in a chain. For example, in the case of Wallet 3 315, the hash value 322 may be displayed publicly with the package identifier technology 333. Package identifier technologies may include: inks, dyes, barcodes, QR codes, microdots, silicon dioxide tags, RFID or IoT devices. This approach cryptographically links a product, to a package, to a database and permits all product/custody information to be recovered from the pC_{DNA} in a product.

Methodologies to link node hashes 320, 321 and 322 are disclosed below.

To sample a product, an application 334 on a 335 computing device provides a user interface that contains modules that:
(i) connect to a computing services platform 336 that implements blockchain processes 337
(ii) process the data streams coming off a package identifier detection device 338 and a oligo sequence detection device 339 and
(iii) decodes the data streams.

A local or remote computing device 335 executes application 334. The computing device 335 is connected to computing services platform 336 that performs blockchain implementation 337.

FIG. 3b is similar to FIG. 3a except that H(C_{DNA}) in each wallet 320, 321, 322 is treated as a separate ‘message header’ to the messages 319. This approach may improve the efficiency in which message information associated with a pC_{DNA} can be recovered from a decentralised, distributed or centralised database. Methodologies to link node hashes 320, 321 and 322 are disclosed below.

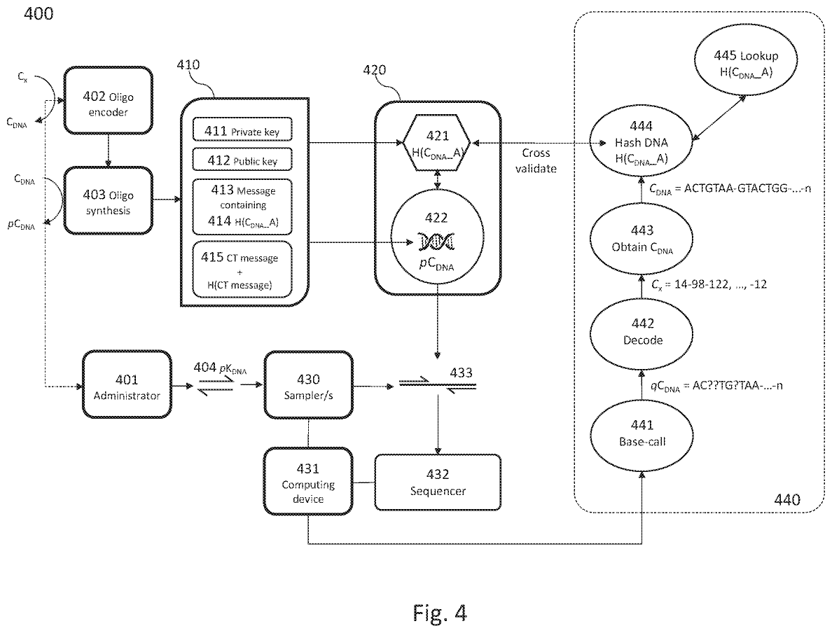

FIG. 4 illustrates a computer system 400 for labelling and sampling a product and illustrates the importance of error detecting and correcting code to compute H(C_{DNA}) to improve security and usability. System 400 uses H(C_{DNA}_A) as a package identifier 421 that can be disclosed to the public (i.e. on a package technology) and as a means to protect the actual DNA code from counterfeiters and also the sampler (which cannot be derived from H(C_{DNA})). H(C_{DNA}_A) is either H(C_{DNA}) or a hash of one or more H(C_{DNA}) optionally concatenated to zero or more of X or a hash of X, where X={a second H(C_{DNA}), time stamp, counter, alternative identifier, random number or padding text}. For illustrative purposes, H(C_{DNA}_A) is a hash of one pC_{DNA} in the sample. The benefits of hashing are covered comprehensively below. One obvious benefit is to protect the actual DNA sequence from all parties for security reasons, and to generate a unique hash value at each node that may also be used as an address to store message data. A brief overview of the sampling, decoding and validation process is now given.

An administrator 401 (or authentication service provider) encodes oligo tags C_{DNA} with an oligo encoder 402. The oligo encoder 402 converts an ASCII codeword Cₓ into a base-4 oligo sequence C_{DNA}. In one example, this involves the use of a 63b RS[9,5]-Ham[7,4] error detecting and correcting codeword flanked by universal primer sites. Error detecting and correcting code is necessary because a single nucleotide error during synthesis or sequencing will completely change the value of H(C_{DNA}) derived from pC_{DNA} in the sample, and give a false-negative product validation.

The physical fragment pC_{DNA} is synthesised by a manufacturer 403 and sent to a product manufacturer 410 who adds the pC_{DNA} to the product 422. The administrator 401 separately sends primer key sequences pK_{DNA} 404 to authorised sampler/s 430. The administrator 401 and/or product manufacturer 410 updates a decentralised, distributed or centralised database with H(C_{DNA}) and associated information.

An oligo manufacturer 403 sends the physical oligo fragment pC_{DNA} together with H(C_{DNA}) to a customer 410. The customer or product manufacturer 410 updates their digital wallet with H(C_{DNA}) information. An example of one process by which a chain is created is shown in the manufacturers wallet 410. Here, the manufacturer uses a private key 411 and public key 412 to create a genesis hash and/or genesis signature of the transaction to start the chain of identity. The public key can be applied to the genesis signature to verify the manufacturer. The manufacturers wallet also includes a message 413 that may contain information such as the batch number, expiry date, manufacturing facility, quality control data, or other. Approaches to transfer the message 413 and H(C_{DNA}) 414 were covered in FIG. 3a and FIG. 3b and are covered in more detail below.

A manufacturer 410 mixes pC_{DNA} into a product 422 which is then packaged 420. The packaged product 420 optionally includes one or more package identifier technologies 421 that contain H(C_{DNA}) information. Methodologies for computing H(C_{DNA}) have been introduced previously and are described in detail below.

To sample, a person 430 tests the product 422 with a computing device 431 connected to a DNA sequencing technology (i.e. DNA sequencer) 432. The computing device 431 may include a computer, laptop or smart phone etc. and has an application downloaded from the administrator as shown in FIGS. 2 and 3a,b.

Before sequencing by the sequencer 432, there may be a polymerase chain reaction (PCR) step 433 where the sampler 430 uses a set of primer keys 404 sent by an administrator 401. In this example, the sequence of the keys is secret, i.e. not known to parties outside the administrator/sampler relationship.

Product validation 440 comprises the following steps. The raw data stream from the sequencer is sent to a server application where it is base-called 441 to obtain a query DNA sequence qC_{DNA}. The query sequence qC_{DNA} will in most cases contain synthesis and sequencing errors. These errors are detected and corrected in the decoding step 442 which gives an ASCII codeword Cₓ. The ASCII codeword is then converted 443 into a corrected DNA codeword, C_{DNA}, and hashed 444 to find a H(C_{DNA}) value. Establishing the correct DNA codeword is of critical importance to the entire system as a single nucleotide error will completely change the value of H(C_{DNA}) and any downstream hashes in a n-ary hash tree. The value of the first level A hash in a hash tree H(C_{DNA}_A) is either H(C_{DNA}) or a hash of one or more H(C_{DNA}) optionally concatenated to zero or more of X or a hash of X, where X={a second H(C_{DNA}), time stamp, counter, alternative identifier, random number or padding text}. For illustrative purposes H(C_{DNA}_A)=H(C_{DNA}) in FIG. 4. The value of H(C_{DNA}_A) derived by sampling 444 is used to validate a product against a hash value store on a database 445 and also used to lookup message information associated with a previously stored values of H(C_{DNA}_A) on a distributed, decentralised, or centralised database. The value of H(C_{DNA}_A) derived by sampling is also used to validate the product by comparing with the value H(C_{DNA}_A) on the package identifier technology 421. The advantage of this system is that the DNA code is secret but can easily be compared to a code on the packaging. The second advantage is that node information contained in a hash tree can be recovered from the product alone.

### Key Properties of Hash(DNA)

The following properties make hash functions useful for this disclosure:

A hash function is deterministic. This means that a hash function applied to any given input string will generate the same output hash value. This property permits product validation by comparing a hash value derived from pC_{DNA} in a product to a hash value stored on a database.

A hash function is irreversible. A hash value is easy to compute for a given input string (ie. DNA sequence), but it is very difficult to find a given input string from a hash value. In other words, for any given hash value it is very difficult to reverse engineer the string of characters that generated it. This quality allows the actual oligo sequence in A, C, G, T to be cryptographically linked to a string of characters (hash). The hash value can be made public, whilst the oligo sequence remains unknown, thereby protecting it against counterfeiters. For example:

Assume a DNA encoding region of length 63 b (RS[9,5]-Ham[7,4] codeword, 7×9=63 b). Also assume a counterfeiter/hacker knows that a DNA codeword is 63 b but does not know the encoding system used, i.e. they know that a DNA codeword C_{DNA} is a Z₄ codeword of length 63 b. Given this information, the hacker knows that the codeword space is 4⁶³=8.5×10³⁷. Also given that the most advanced 8× Nvidia GTX 1080 Hashcat systems can brute force 330 GB hashes s⁻¹ and assuming that on average 50% of the codeword space is brute forced before a solution is found, then the expected time to solve a hash is E(solved)=4.1×10¹⁸ years (˜280 million times longer than the universe has existed). Therefore, it is safe to use H(C_{DNA}) or H(C_{DNA}_A) as a package identifier. In FIG. 4:
Cₓ=14−98−122, . . . , −127: an ASCII codeword, ie. RS[n, k] in GF(128)
C_{DNA}=SEQ ID NO: 1 : a DNA codeword, i.e. RS[n, k]-Ham[n, k]
H(C_{DNA}) =2ced98d5e43a193 . . . (SHA-256)

A single change in the input string generates a completely different hash value. This property stops a potential hacker changing a record in a hash tree. It also prevents counterfeiting by generating a similar oligo sequence.

It is infeasible to find two different strings with the same hash value. This quality ensures that each pC_{DNA} generates a unique hash value, ie. hash values of two different pC_{DNA} are extremely unlikely to collide (ie. be the same). In some examples, where a hash value is of a length that is shorter than the oligo sequence, collisions (two different DNA sequences that generate the same hash value) are possible but extremely unlikely. Nevertheless, it is noted that collisions do not significantly affect the working of the disclosed solution because the obfuscation of the DNA sequence remains the two identical hashes for different DNA sequences can be permitted in the system for look-up purposes. On the other hand, current computer systems are well capable of calculating hashes that are longer than the used DNA sequences and therefore, collisions should not occur in practical implementations. Additionally, the incidence of collisions may be reduced by hashing a concatenation of H(C_{DNA}) with one or more of X or a hash of X, where X={a second H(C_{DNA}), time stamp, counter, alternative identifier, random number or padding text}.

In this disclosure we favour cryptographic hash functions and keyed cryptographic hash functions for the reasons given above. A non-exhaustive list of these functions include: BLAKE-256, BLAKE-512, BLAKE2, BLAKE2s, BLAKE2b, ECOH, FSB, GOST, Grostl, HAS-160, HAVAL, HMAC, JH, MD2, MD4, MD5, MD6, One-key MAC, Poly 1305-AES, PMAC, RadioGatún, RIPEMD, RIPEMD-128, RIPEMD-160, RIPEMD-320, SHA-1, SHA-224, SHA-256, SHA-384, SHA-512, SHA-3, SipHash, Skein, Snefru, Spectral Hash, Streebog, SWIFFT, Tiger, UMAC, VMAC, Whirlpool. In this document, the term ‘hash’ and ‘hashing’ refers to all hash function variants including: cyclic redundancy checks, checksum functions, hash functions, cryptographic hash functions, and keyed and unkeyed cryptographic hash functions.

To those skilled in the art, it is known that the conversion of Z₄ oligonucleotide text into ciphertext can be achieved using a wide variety of encryption methodologies such as: shift ciphers, substitution ciphers, Vigenere ciphers, permutation ciphers, stream ciphers (for e.g. the Lorenz cipher, Linear feedback shift registers, LFSR), block ciphers (Feistel, DES, Rijndael), message authentication codes (e.g. HMAC) public key encryption (e.g. RSA, El Gamal, Rabin, Paillier), and others.

It is noted that the system described above allows the identification of a product that originates from a single manufacturer by the manufacturer creating the DNA sequence adding it to the product and calculating a hash value for it. The recipient recovers and decodes the DNA sequence/s in a product, hashes them, and compares the derived hash value to the hash from the manufacturer/s. FIG. 3 already foreshadowed that there may be multiple parties that each contribute to the supply chain either by further manufacturing the product (refining, mixing, etc.) or checking the product quality. The following description provides further information about the use of the disclosed system to verify products in such a multi-entity supply chains.

### Oligonucleotide Fragment Design Approaches

Here, three main approaches to record supply chain information into oligonucleotide tags are disclosed. Three distinct pieces of information are needed to recover a chain of identification/custody/provenance from a product in a supply chain:
(i) Information that identifies the product
(ii) Information that identifies the nodes in a supply chain,
(iii) Information that gives the order of nodes in a supply chain

Here, three broad approaches to store product, node identification, and node order information in pC_{DNA} tags in a product are disclosed. These methodologies all permit transactions that are recorded in a virtual blockchain to be mirrored or partially mirrored in a physical oligonucleotide ‘blockchain’ that is integrated into a product. It should be appreciated that this disclosure covers all variants of these methodologies.

In a first approach (Oligonucleotide Tag Methodology 1, OTM1) oligonucleotide tags identify the node at which they are added only, and the order is stored on a distributed, de-centralised, or centralised database as a chain or tree of hashes.

In a second approach (Oligonucleotide Tag Methodology 2, OTM2) oligonucleotide tags contain a placement identifier that includes information about a node and the position of a node in a supply chain.

In a third approach (Oligonucleotide Tag Methodology 3, OTM3) oligonucleotide tags contain node information and are sequentially ligated to oligonucleotide tags that already exist in the product using a ligation reaction (for e.g. PCR). The growing oligonucleotide chain stores both order and node information.

Two main classes of oligonucleotide tag are introduced in the descriptions of OTM1-3 below. The first is a product unique identifier denoted by C_{DNA}UI_n. The second is a node unique identifier denoted by C_{DNA}UI_n. Both C_{DNA}UI/NI oligonucleotide tag variants may be hashed together and cryptographically linked to a unique package identifier denoted by PI.

### Oligonucleotide Tag Methodology 1 (OTM1)—Node Identification Information is Stored in the Oligo Tag, Node Order Information is Stored Remotely

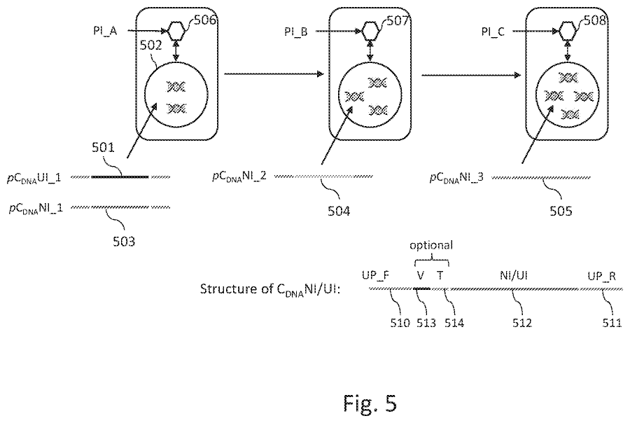

FIG. 5 is a diagram of OTM1 that, for illustrative purposes, uses pC_{DNA} tags only. First an oligonucleotide fragment encoded with a unique product identifier (pC_{DNA}UI_1) 501 is added to the product 502. Optionally a second oligonucleotide fragment with node identification information pC_{DNA}NI_1 503 is also added to the product. A hash that includes a hash of one or both fragment codewords H(C_{DNA}UI/NI) may be displayed on the package using a package identification technology 503 such as those listed previously.

Additional pC_{DNA} tags may be added along the supply chain to record an event that occurs at a node, such as a quality control step 504, 505. These tags identify the node and may be considered as an analogue to a public key in DNA. In FIG. 5 these tags are marked pC_{DNA}NI_1-3 503, 504, 505. The PI code may be updated as new pC_{DNA} are added and the product is repackaged or recombined 507, 508. This capability is particularly important for ingredient tracing, upstream from the point of finished product manufacture.

FIG. 5 also shows a structure of the oligonucleotide tag where UP_F 510 is a universal forward primer site, UP_R 511 is a universal reverse primer site, and UI/NI 512 is a unique sequence that identifies a product or node, respectively. The oligonucleotide tag may optionally include a sub-sequence V 513 that identifies the version of the DNA encoding system used. Furthermore, the oligonucleotide fragment may optionally contain an additional subsequence T 514 that distinguishes a UI tag from a NI tag. These optional sub-sequences may improve testing efficiency, genesis hash validation efficiency, and help identify the oligonucleotide encoding system used.

In OTM1 the order in which the oligo tags are added cannot be derived from the product alone. Therefore, this approach uses an external system to store the order, in this case a series of hash values of the pC_{DNA} added to a product (see below). The order is found by iteratively computing node hash values from the pC_{DNA} in product sample and cross-validating values stored on a distributed, decentralised, or centralised database.

The advantage of OTM1 over OTM2 is that one pC_{DNA}NI code is used per node across different batches and different products. In OTM2 multiple pC_{DNA}NI are used at each node that contain different order information, which may be cumbersome and increase the risk of a node member adding a fragment with incorrect placement information.

The advantage of OTM1 over OTM3 is that OTM1 does not rely on a node member returning ligated oligonucleotide tags backs to a product. The optimal approach is likely dependent on a particular application.

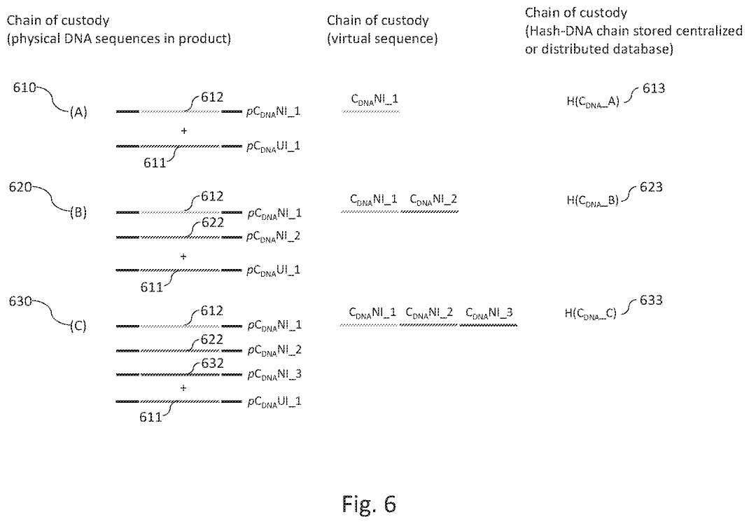

FIG. 6 illustrates the operation of a chain of custody in pC_{DNA}. This figure shows three nodes 610, 620 and 630 in a supply chain. At the first node 610 two tags are added, one that contains a product unique identifier pC_{DNA}UI_1 611 and one that contains a node identifier sequence pC_{DNA}NI_1 612. The manufacturers node identification information is stored in a hash chain or tree H(C_{DNA}_A) 613 that includes a hash of pC_{DNA}UI_ and pC_{DNA}NI_1. At the second and third nodes 620, 630 downstream product manufacturers may want to combine products of a different origin (i.e. blend different cannabis oils to get the correct cannabinoid content) or perform a quality control step (i.e. test the cannabinoids). This step may be certified with their respective node identifiers 622, 632 and a hash of these identifiers can be added to a tree of hashes H(C_{DNA}_B) 623 and H(C_{DNA}_C) 633. Other message information may be appended to the node hash values 613, 623 and 633.

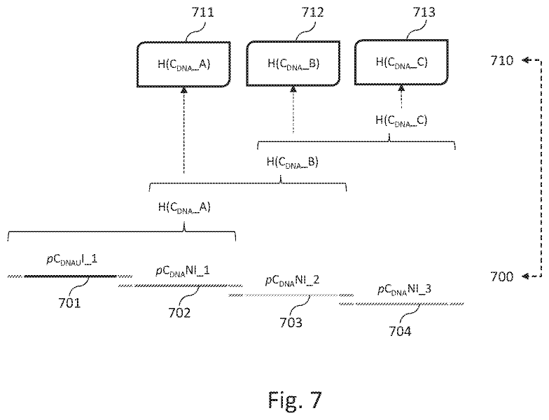

FIG. 7 shows an example of how a physical chain of information stored in oligonucleotide fragments may be linked to a virtual chain of information transacted between digital wallets using OTM1 and a binary tree of hashes. In this example the pC_{DNA} in a product 700 is cryptographically linked to a virtual chain of identification/custody that is stored in a distributed, decentralised, or centralised database 710. A hash of each pC_{DNA} 701, 702, 703, 704 added to the product may be used as input to compute a binary tree of hashes, where each node hash value is a cumulative hash with two inputs. The first input includes a value that identifies previous transactions in a chain, the second input includes new information added at a node. Node hash values are computed and stored in the wallets 711, 712, 713, or in a distributed ledger, as a virtual chain/tree of hashes. These hashes are comprised of a series of hashes of C_{DNA}NI/UI codewords that are encoded into the physical oligonucleotide tags pC_{DNA}NI/UI. This example shows a binary tree approach that sequentially links a cumulative node hash to a hash of the next pC_{DNA} added, although other approaches may be taken (see below). The order of 700 is reconstructed from a sample by brute forcing all possible node hash values from the pC_{DNA} detected in a sample to values stored on a database. This means the final hash value is calculated for each of multiple combinations of the available upstream node hash values and the result is compared to the database noting that only one combination should provide a match.

This brute force calculation of all combinations may become infeasible for a large number of nodes with potentially branching and merging paths. As a computationally more efficient alternative, it is possible to identify an upstream node for which the hash value matches. For example, binary pairs of only two hash values can be computed and they should match to one of the very first nodes. From there, the process can iteratively step downstream so that at each step only combinations between the current chain hash and all individual hashes need to be computed. The result should be linear in complexity compared to exponential complexity of the brute force option above. In examples where the hash values are based on additional data, such as product identifiers, entity identifier, etc., the sampled hash value can be iteratively tested against different combinations of the additional data to validate a match on the database.

Furthermore, as described below, a hash value at any node can either be H(C_{DNA}UI/NI) or a hash of one or more H(C_{DNA}UI/NI) optionally concatenated to zero or more of X or a hash of X, where X={a second H(C_{DNA}UI/NI), time stamp, counter, alternative identifier, random number or padding text}. As also described previously, a node hash may be displayed publicly using a package identifier technology.

In summary, in OTM1, a ‘fingerprint’ of custody (without order) is stored in the product as a set of encoded oligonucleotide fragments pC_{DNA}NI/UI. The order in which fragments are added to a product, is stored as a list of tree of hashes remotely. The order can be iteratively reverse-engineered from the pC_{DNA} fragments detected in a sample through brute forcing and cross validation of generated hash values.

Methodologies for hashing at a discreet node, and between nodes, are described below.

### Oligonucleotide Tag Methodology 2 (OTM2)—Node Identification Information and Order Information is Stored in the Tag

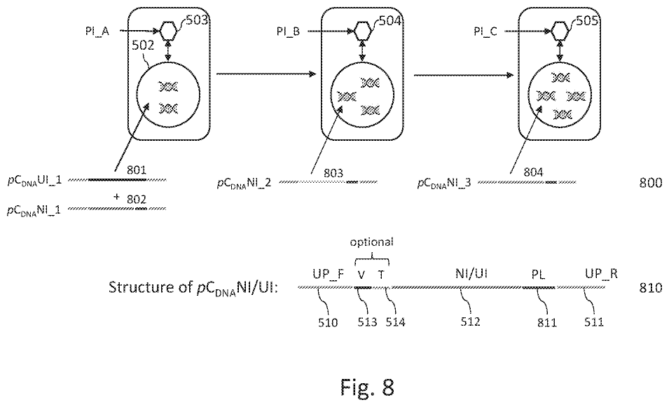

FIG. 8 shows oligonucleotide tag methodology 2 (OTM2). First an oligonucleotide fragment encoded with a unique product identifier (pC_{DNA}UI) 801 is added to a product 502. The subsequent pC_{DNA} added to the product identify the node pC_{DNA}NI and additionally contain a ‘placement identifier’ sub-sequence (PL) 811. The placement identifier 811 is used to reconstruct the order in which the tags are added to a product. This allows a chain of custody/information to be established from the pC_{DNA} fragments in a product alone. A hash that includes a hash of the first identifier H(C_{DNA}UI), or any node hash value in a hash chain/tree, may be displayed on the package using a package identification technology 503 such as those listed previously.

### Notes on OTM2

OTM2 permits supply chain node information and order to be recovered from the product alone. However, each node requires multiple different tags (pC_{DNA}NI) with different placement identifiers, and these must be used correctly.

The advantage of OTM2 over OTM1 is that supply chain node and order information is recoverable from a product alone.

The advantage of OTM1 over OTM3 is that sampled and ligated product does not have to be returned to the product to mark that a particular event has occurred.

FIG. 8 also shows the design of an oligonucleotide tag used in OTM2. Again, UP_F 510 is a universal forward primer site, UP_R 511 is a universal reverse primer site, and UI/NI 512 is a unique codeword that identifies a product or node, respectively. In OTM2 a node placement identifier PL 811 is also required. The exact placement of 811 can be anywhere between 510 and 511. The oligonucleotide tag may optionally include a subsequence V 513 that identifies the version of the DNA encoding system used. Furthermore, the oligonucleotide fragment may optionally contain an additional subsequence T 514 that distinguishes a UI tag from a NI tag. These optional sub-sequences may improve testing efficiency, genesis hash validation efficiency, and identify the oligonucleotide encoding system used.

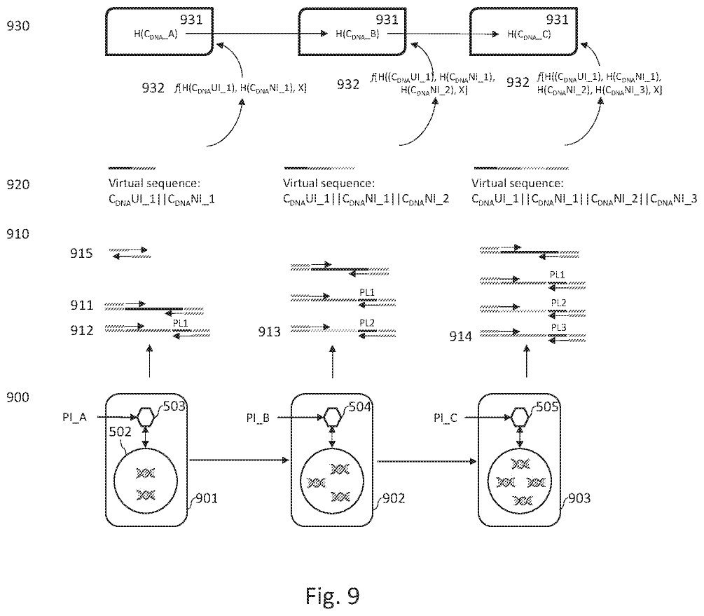

FIG. 9 illustrates how a chain of identification is recovered from the product using OTM2. The concept is similar to OTM1, except that the pC_{DNA}NI contain an additional subsequence that identifies a node's placement in a supply chain. In this example a single product 900 is labelled at three different nodes in a supply chain 901, 902, and 903. The product at node 1 901 contains one product unique identifier sequence pC_{DNA}UI_1 911 and one node identifier sequence pC_{DNA}NI_1 912 that includes the placement identifier PL1. The product at node 2 is labelled with one additional node identifier sequence C_{DNA}NI_2 913 that includes the placement identifier PL2. The product at node 3 is labelled with a third node identifier sequence C_{DNA}NI_3 914 that includes the placement identifier PL3. These sequences may be cryptographically linked to a package identifier (PI) technology 503, 504, 505 and displayed on the packaging. A hash of a node identifier HC_{DNA}NI) may be thought of as a node public key.

Supply chain information is recovered from the product by first reacting a sample of the product with a secret set of primer keys pK_{DNA} 915 in a PCR reaction. The use of universal primer sites and in some cases identical encoding region sub-sequences may cause cross-fragment hybridisation. This problem is addressed using a technique called annealing temperature discrimination PCR (ATD PCR), which was disclosed in PCT/AU2017/050757 filed on 21 Jul. 2017 and entitled “A METHOD FOR AMPLIFICATION OF NUCLEIC ACID SEQUENCES”. ATD PCR allows any set of pC_{DNA} at a node in 910 to be amplified in only one reaction.

A placement identifier subsequence (PL) permits the order in which each separate pC_{DNA}NI is added to be reconstructed from the product alone. In 920, for example, the OTM2 fragment order is shown as a concatenation (∥) of C_{DNA}UI and C_{DNA}NI for illustrative purposes. At node 3 in 920 the order is given as C_{DNA}UI_1∥ C_{DNA}NI_1∥ C_{DNA}NI_2∥ C_{DNA}NI_3. As covered previously, and shown in 930, hashes of the pC_{DNA} in the product and elements of the set X 932 may be used to store node information in a distributed, decentralised or centralised database 331 that is either managed by members of the supply chain or an administrator, or a combination of the two.

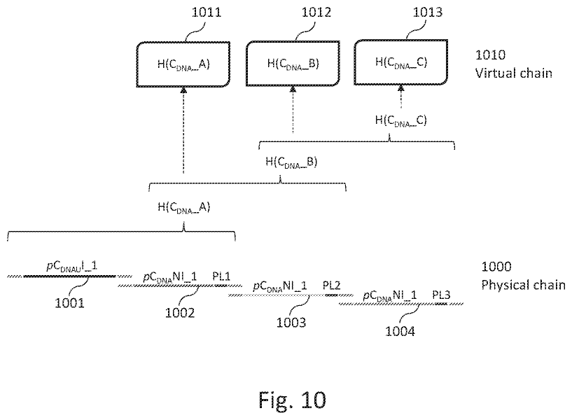

FIG. 10 shows how supply chain information encoded into a physical oligonucleotide chain in OTM2 1000 may be cryptographically linked to supply chain information stored in a distributed, decentralised, or centralised database 1010. In this example, the product is labelled with one pC_{DNA}UI 1001 and one pC_{DNA}NI 1002 that contains a placement identifier subsequence at node 1. At each other node the product is labelled with one pC_{DNA}NI 1003, 1004. The physical chain is reconstructed form the placement identifier sub-sequences PL-13 in the pC_{DNA}NI. In this example the hashes of each pC_{DNA} added to a product are sequentially computed in a cumulative binary tree 1010 stored in a distributed, decentralised or centralised database. Node hash values may be computed from the pC_{DNA} in a product and validated against database that contains node hash values and addition node message information.

As described below, the hash at any node for OTM2 can either be H(C_{DNA}UI/NI) or a hash of one or more H(C_{DNA}UI/NI) optionally concatenated to zero or more of X or a hash of X, where X={a second H(C_{DNA}UI/NI), time stamp, counter, alternative identifier, random number or padding text}. Different methodologies to cryptographically link the nodes together are disclosed below (in FIG. 10 a binary tree structure is shown). Lastly, the hash value at each node may be displayed publicly with a package identifier technology, and used to lookup other associated message information.

The advantage of OTM2 over OTM3 is that purified oligo tags are added rather than ligated to existing product tags from a testing step. The use of ligated product tags may be problematic in some applications. For OTM2 the (1) amount and purification standards of the additional oligo tag can easily be controlled, and (2) the system does not rely on node members performing the more complex steps of OTM3, described below, correctly.

### Oligo Tag Methodology 3 (OTM3)—Oligo Tags Contain Node Information, Order is Stored by Sequentially Ligating of Node Identifier Sequences to the pC_{DNA} in a Product

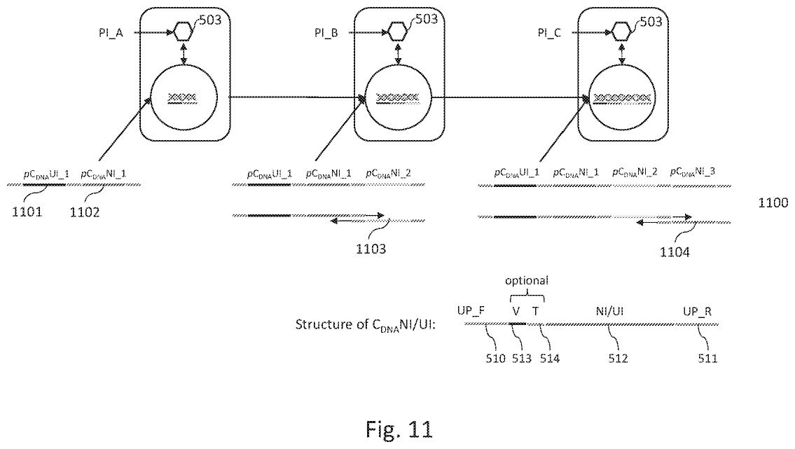

Oligonucleotide tag methodology 3 (OTM3) comprises a physical oligonucleotide ‘blockchain’ that is progressively written into a growing oligonucleotide fragment at each node using a concatenation reaction to ligate additional pC_{DNA}. FIG. 11 illustrates OTM3, where the order in which the node identifier fragments pC_{DNA}NI are added is recorded in a growing DNA strand by sequential ligation (for example, PCR or other ligation reaction). At each step, a node member takes a sample of the product, ligates their own pC_{DNA}NI (1102, 1103, 1104) to the pC_{DNA} already in the product, and returns the ligated oligonucleotide fragment back to the product. In this way, information about a node, as well as the order in which a node identifier tag is added, is written into an oligonucleotide strand that is reincorporated into the product. The ligation steps can be thought of as a series of pC_{DNA} concatenation steps. As described previously the cumulative hash at each node may be displayed as a unique package identifier (503, 504, 505).

The structure of the oligo tags used in OTM3 is similar to that disclosed in OTM1 TMI and comprises 510, 511, 512, 513, 514, except that one of the primer keys pK_{DNA} contains pC_{DNA}NI 1102, 1103, 1104 or the reverse complement sequence of the pC_{DNA}NI. The second pK_{DNA} is a universal primer sequence that permits an exponential polymerase chain reaction when used in combination with the first pK_{DNA} that contains pC_{DNA}NI.

### Notes on OTM3

In OTM3 supply chain information is stored in the oligonucleotide tags by physically concatenating the pC_{DNA} tags together. This approach requires a node member to sample an incoming product, perform a ligation reaction with their pC_{DNA}NI, and return the product of the ligation reaction back to the product.

The advantage of OTM3 over OTM1 is that all supply chain information is recoverable from the product (order+node information).

The advantage of OTM3 over OTM2 is that there is no need to issue multiple public keys to each node that contain different placement identifiers.

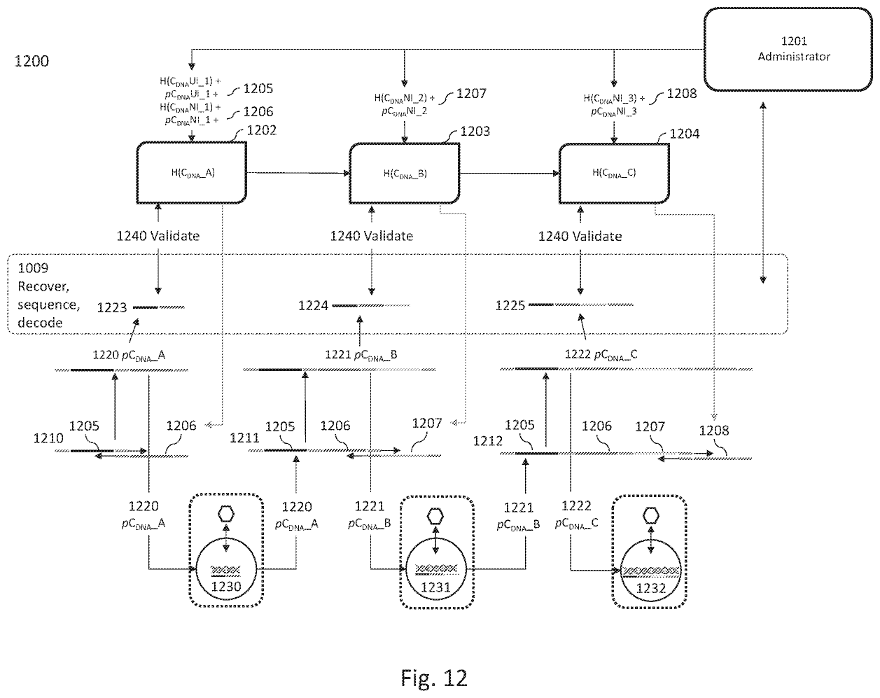

FIG. 12 illustrates how supply chain information is encoded into oligonucleotide fragments and used to label a product with OTM3. The node identifiers C_{DNA}NI may be viewed as analogous to a public key.

In the example in FIG. 12, the system 1200 comprises an administrator 1201 that sends a set of primer keys to node members 1202, 1203, 1204 that comprise a first universal primer sequence and second primer sequence that contains node information C_{DNA}NI) 1206, 1207, 1208. The members and their digital wallets are represented at 1002, 1003, 1004. The genesis node in this case is node 1202. The administrator also sends a hash of C_{DNA}NI/UI to the node members so that the actual oligonucleotide sequence is not disclosed. A product unique identifier pC_{DNA}UI_1 1205 is also sent to 1202.

The digital wallets include node hash values H(C_{DNA}_A-C) derived from the pC_{DNA} added at each node, and optionally additional information from the set X, as described previously. In this example the genesis hash at 1002 is H(C_{DNA}_A), and the node hash at 1003 is H(C_{DNA}_B) and at 1004 is H(C_{DNA}_C). Node hashes link the chain of information stored in the physical oligonucleotide fragments pC_{DNA}UI/NI to the virtual chain of information stored in a distributed ledger or other database. Thus, a virtual chain of custody is mirrored by a physical chain of custody, which is integrated into a product.

In the example in FIG. 12, the first member 1202 ligates their node identifier pC_{DNA}NI_1 1206 to a product unique identifier pC_{DNA}UI_1 1205 in the reaction 1210. Note that other genesis hash variants described below are also possible. The resulting concatenated oligonucleotide fragment pC_{DNA}_A 1220 is used to label the product 1230. The equivalent concatenated sequence is 1223. A hash of a hash of the fragments 1205 and 1206 may be used to compute the genesis hash at node 1002 H(C_{DNA}_A), optionally in place of, or together with, zero or more elements in the set X. To sample, a concatenated oligonucleotide fragment pC_{DNA}_A 1220 is recovered from a product, optionally amplified by PCR, and used to recover supply chain information by computing and cross-validating H(C_{DNA}_A) 1240 derived from the sample with H(C_{DNA}_A) stored in a virtual environment such as a distributed, decentralised, or centralised database.

In the second step, a member at node 2 1203, recovers a sample of the concatenated pC_{DNA}_A oligonucleotide from the product 1230, and ligates their own node identifier sequence C_{DNA}NI_2 1207 in reaction 1211. The resulting oligonucleotide strand pC_{DNA_}B 1221 now contains node/custody information about node 2, and is used to label the product 1231 at node 2. The resulting oligonucleotide may also be used to validate 1240 the received product by computing a hash of the previous C_{DNA}_UI/NI in the sample.

Similarly, in the third step a member at node 3 1204, recovers a sample 1221 of the concatenated pC_{DNA}_B oligonucleotide from the product 1231, and ligates their own node identifier sequence C_{DNA}NI_2 1208 in reaction 1212. The resulting oligonucleotide strand pC_{DNA}_C 1222 now contains node/custody information about node 3, and is used to label a product 1222 at node 3. To sample, the resulting oligonucleotide may also be used to validate 1240 the received product by computing a hash of the previous C_{DNA}_UI/NI in the sample.

As in OTM1 and OTM2, process described above for OTM3 at nodes 1202, 1203, 1204 may continue for an unlimited number of nodes.

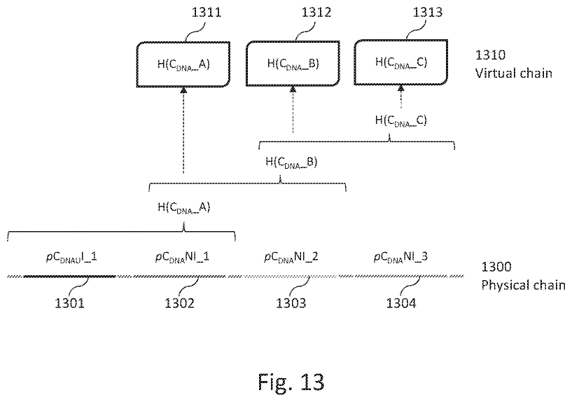

FIG. 13 shows how the physical oligonucleotide fragment/s in methodology 3 (OTM3) 1300 are cryptographically linked to a virtual chain of identification/custody that is stored in a distributed, decentralised, or centralised database 1310. Note that the physical sequence is one fragment 1300 that is comprised of a series of ligated pC_{DNA}UI/NI sub-sequences 1301, 1302, 1303, and 1304. In this example, the hashes at each node 1311, 1312, 1313 are similarly comprised of a series of hashes of the C_{DNA}NI/UI physical tags pC_{DNA}NI/UI 1301, 1302, 1303, and 1304. As described below, the hash at any node can either be H(C_{DNA}UI/NI) or a hash of one or more H(C_{DNA}UI/NI) optionally concatenated to zero or more of X or a hash of X, where X={a second H(C_{DNA}UI/NI), time stamp, counter, alternative identifier, random number or padding text}. Different methodologies to cryptographically link the nodes together are disclosed below. In the example in FIG. 13 a binary tree structure is shown. Lastly, the hash value at each node may be displayed publicly with a package identifier technology.

The steps above result in an immutable chain identification/custody that is written into a physical growing DNA strand 1300 returned to the product. Note that when a chain of custody is written into the growing oligo fragments, the order matters. ATD PCR (disclosed in PCT/AU2017/050757 filed on 21Jul. 2017 and entitled “A METHOD FOR AMPLIFICATION OF NUCLEIC ACID SEQUENCES”) may be used to minimize cross-hybridization between multiple different fragments containing common primer sites or common sub-sequences. Due to the property that hash functions are deterministic, an entire supply chain may be validated by comparing the hash of the final concatenated pC_{DNA} fragment 1300 to the hash of the supply chain.

To those skilled in the art, it will be appreciated that a two-step reaction may be used to sample and label a product with OTM3. In the first step, an oligonucleotide fragment is amplified in a PCR reaction, and the amplified PCR product is both used (1) to validate the sample, and (2) as a substrate in a second ligation reaction where subsequent node/chain of custody information is concatenated. It will also be appreciated that a ligation reaction refers to any reaction that results in a concatenated oligonucleotide fragment.

### Methodologies to Cryptographically Link C_{DNA}UI/NI at and Between Nodes in a Network

In this section, different methodologies to encrypt DNA codewords C_{DNA}, and cryptographically link pC_{DNA} at nodes and between nodes is disclosed. These approaches are used to: (1) protect an oligonucleotide codeword C_{DNA}, (2) protect against data hacking/tampering, (3) generate a unique cumulative cryptographic signature at each virtual node that can be computed from the physical pC_{DNA} in a product for validation purposes, (4) generate a unique cumulative cryptographic signature at each virtual node that can be used append and lookup other message information, and (5) generate a unique cumulative cryptographic signature at each virtual node for the purpose of reverse-engineering the order in which the oligonucleotide tags are added along a supply chain (for OTM1).

### Key Capabilities and Characteristics of a Secure Oligo Tracing System

First, the key capabilities and properties of a secure oligo-encryption system are summarised:

The oligo tag sequence, C_{DNA}, is protected.
The order in which C_{DNA} is added is stored or derived from C_{DNA}.
The C_{DNA} are stored securely and in a structure that protects against tampering
The order and node information associated with each pC_{DNA} added to a product is derivable from information contained in an unpackaged product
The order and node information associated with each C_{DNA} added to multiple products that are mixed in unknown ways can be recovered from an unpackaged product (i.e. an address within a hash chain/tree is recoverable from the product):
Supply chain should be computable from the pC_{DNA} in a sample, together with a value that identifies each node in a supply chain.
A value that identifies each node in a supply chain may be used as an identifier to attach additional node information (for e.g. batch number, expiry date, manufacturing facility, manufacturer, time stamp, product safety information, quality control information, etc.).
Supply chain information should be recoverable from an unpackaged product, with the correct permissions.
As few C_{DNA} tags as possible should be added to a product.

### Why Hash C_{DNA}?

Hashing is often described as the work horse of cryptography. For this disclosure, hashing offers the following:
Protects a oligonucleotide codeword C_{DNA} against counterfeiters
A way to link the C_{DNA} codewords together, in a similar way to a block chain, to protect against attacks on data stored in distributed, decentralised or centralized database
Permits a record of the order in which pC_{DNA} tags are added to a product (for OTM1)
Links a chain of two or more C_{DNA} that only one unique C_{DNA} is required to compute a tree of unique hash values for each product
Links two or more C_{DNA} so that a combined hash node value is unique and searchable: and
Generates a unique hash record of a supply chain that may be reconstructed from the set of pC_{DNA} in an unpackaged product, even if tagged products are split or mixed.

### Hash Methodologies at and Between Nodes

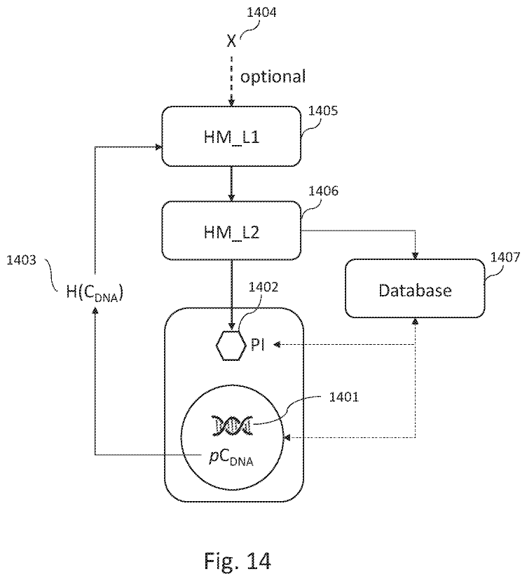

Methodologies for hashing C_{DNA} codewords at discreet nodes and between nodes are now disclosed. FIG. 14 illustrates a pC_{DNA} tagged product 1401 that is packaged with a unique package identifier 1402. Hash values at each node 1405 (hash methodology level 1, HM_L1) and between nodes 1406 (hash methodology level 1, HM_L2) are comprised of, or derived from, one or more oligonucleotide codes C_{DNA} 1403 and optionally zero or more of the set X 1404.

The set X 1304 includes: {a second H(C_{DNA}), alternative identifier or H(alternative identifier), time stamp or H(times tamp), counter or H(counter), random number or H(random number), or padding text or H(padding text)}. The terms in the set X are defined as follows:
C_{DNA}=a Z₄ codeword comprised of {A, C, G, T, and/or optionally U} that represents either a node identifier C_{DNA}NI) or unique product identifier (C_{DNA}UI) codeword.
Alternative identifier (Alt_ID)=a string of ASCII text that is analogous to a public key (PbK) or hash of a private key H(PvK) that is not directly associated with any C_{DNA}.
Time stamp=a record of the time and date, updated at a defined interval.
Counter=a counter that is arbitrary and updated at a defined interval.
Padding text (Pₙ)=ASCII text optionally used to pad a C_{DNA} codeword. Padding text lengthens the codeword and may thereby reduces the incidence of collisions and improve security.

Concatenated text (∥) is text linked together in a series or a chain, and a hash function applied to an input is denoted by as H(input) throughout this document. A package identifier is denoted by PI and may be cryptographically linked to the pC_{DNA} in a product through a hash value computed at a node in tree of hashes that represent events in a products supply chain. A package identifier may alternatively be liked to a hash tree via a proxy identifier that points to a hash value at a node in a hash tree.

The different hash methodologies used at the level of the node (HM_L1) 1405 and between nodes (HM_L2) 1406 are now disclosed with reference to FIG. 14. In. FIG. 14 HM_L1 1405 includes hash methodologies at each individual node (HM_L1) and HM_L2 1406 includes methodologies in which node hashes are linked together.

Hash methodologies at each node (HS_L1, level 1) 1405 can be nested, and can take the form of any concatenation of a C_{DNA} with X in any order. The following non-exhaustive list gives examples of HM_L1 hashes. The examples in FIGS. 7, 10 and 13 used the a binary genesis hash with two C_{DNA} inputs: H[H(C_{DNA}_1)∥H(C_{DNA}_2)]. Note that a node hash may not contain a H(C_{DNA}), but a cumulative hash must derive from at least one H(C_{DNA}). Hash methodologies at each node (HS_L1, level 1) include:
H(C_{DNA})
H(X)
H[H(C_{DNA}1)∥H(C_{DNA}2)]
H(X1∥X2)
H[X∥H(C_{DNA})]
H[X₁∥X₂∥ . . . Xₙ∥H(C_{DNA})]
Or any combination of the above.

Hash methodologies at each node (HM_L1) may be linked together with level 2 hash methodologies HM_L2. Note that all HM_L2 hashes derive from or include one or more H(C_{DNA}) incorporated at a previous node. Level 2 hash methodologies include:
A list of previous hashes in the chain/tree that are not linked: e.g. H(C_{DNA}_1), H(X_1), H(C_{DNA}_2), H(X_2), . . .
A list of previous identifiers that are concatenated and then hashed: H(C_{DNA_}1∥X_1∥C_{DNA_}2∥X_2, . . . )
A binary tree of hashes where a new node hash value is a hash of the previous node hash value concatenated one element of the set X. For example:
   Node 1: H(A)=H[X₁∥H(C_{DNA}_1)]
   Node 2: H(B)=H[H(A)∥H(X₂)], no pC_{DNA} added to product
   Node 3: H(C)=H[H(B)∥H(C_{DNA}_2), an additional pC_{DNA} added to product
An-ary tree of hashes where a new node hash value is a hash of the previous node hash value concatenated one or more elements of the set X.
A Merkle tree.

For illustrative purposes, the following sections mostly refer to Oligo Tag Methodology 1 OTM1 in combination with a binary tree hash approach as shown in FIG. 7.

### Genesis Hash

A genesis hash is the first hash in a chain or tree of hashes. If hashes are linked in a tree, a change in one input hash value will change the value of all downstream node hash values. This means that a change in one input C_{DNA} value is transferred to all downstream nodes in a product's supply chain. The implication is that if one element of a genesis hash is unique to a supply chain, all downstream node hash values will also be unique.

The propagation of different node hash values down a chain or tree of hashes from a single changed input permits (1) node identifier codewords to be re-used (pC_{DNA}NI), and (2) other product information to be to be attached to a distinct node hash value (for e.g. quality control, custody, timestamp et.) and stored in database. This means that fewer unique pC_{DNA} need to be issued to mark that a particular event has occurred. Rather than changing all of the tags in a product, nested hashing allows only one element in the tree to be changed, such that this change is transferred to all downstream nodes in the tree. The following disclosure provides six examples for creating a unique genesis hash.

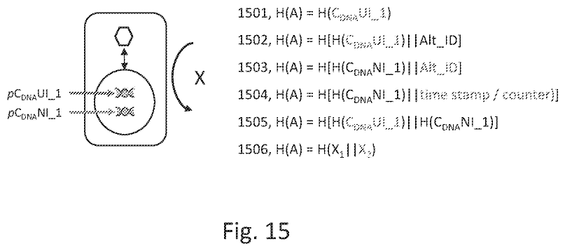

FIG. 15 illustrates six different approaches to generating a genesis hash, H(A). To create a unique genesis hash, at least one element of the hash must be unique to a particular item/batch/product. In the examples 1501, 1502, 1503, 1504, 1505 and 1506 the unique element is shown in red and may include an element of the set X (see above). A hash of an individual element is only required if that element should be kept secret. In the examples disclosed herein only hashes of each C_{DNA} element are shown. Note that n-ary nested tree hashing approaches allow the same node identifier C_{DNA}NI to be re-used across different batches and, therefore, C_{DNA}NI is not used as a unique element (for OTM1). The ‘1’ indicates the first node, where a genesis hash is computed, H(A).

In the first example 1501 a genesis hash H(A) is simply a hash of a unique oligonucleotide product identifier, H(C_{DNA}UI_1).

In a second approach 1502 a genesis hash H(A) is a hashed concatenation of a hashed unique product identifier and alternative identifier, H[H(C_{DNA}UI_1)∥X]. Here, X=Alt_ID may be a fixed value that can be thought of as a ‘public key’ that identifies the node. The advantage of this approach is that only one C_{DNA} is used to generate H(A), and H(A) contains node information. The genesis hash is identified from a product sample by finding the hash of each C_{DNA}UI in a sample and computing all possible H(A) against a database of Alt_ID/public keys until a match is found, ie. H(A)sample=H(A)database.

In a third approach 1503 a genesis hash H(A) is a hashed concatenation of a hashed node identifier and X where X=alternative identifier, H[H(C_{DNA}NI_1)∥X]. Here the value of H(C_{DNA}NI_1) can be thought of as a ‘public key’ that is fixed and reused across different products/batches/items/transactions at same node. Unique information about the product or batch is stored in the alternative identifier that changes with each product/batch. The genesis hash is recovered from a sample by finding each HC_{DNA}NI) in the sample and computing each H(A) with a database of X alternative identifiers until a match is found, ie. H(A)ₛₐₘₚₗₑ=H(A)_{database}.

In a fourth approach 1504, a genesis hash H(A) is a hashed concatenation of a hashed node identifier and X where X=time stamp, counter or random number: H[H(C_{DNA}NI_1)∥X=TimeStamp/counter/random number]. In this approach a time interval should be set so that it is sufficiently short to capture a single transaction, but sufficiently long so that a suitable number of hashes is generated over a specified time period to permit decoding. For example, if the TimeStamp is set to an interval of one minute, and assuming a time period of 10 years, 5,256,000 genesis hash values are possible. Given a hash mining rate of 330 B hashes s⁻¹, and assuming there are 10 pC_{DNA}NI in a sample, the expected time to compute and validate the genesis hash from a sample is <0.0001 seconds.

In a fifth approach 1505, a genesis hash H(A) is a hashed concatenation of a hashed C_{DNA} product unique identifier and a hashed C_{DNA} node identifier, H[H(C_{DNA}UI_1)∥H(C_{DNA}NI_1)]. In this approach two C_{DNA} tags are added to the product to generate H(A). The genesis hash is recovered from a sample by computing every combination of possible genesis hashes in the sample, i.e. every combination of H(C_{DNA}UI) with each H(C_{DNA}NI) detected, and cross validating the resulting values against a database of genesis hash values.

Lastly, in a sixth approach 1506, a genesis hash is a hashed concatenation of X₁ and X₂ and does not contain a H(C_{DNA}). In this approach X₁ is variable and identifies a product or batch number and X₂ is constant and identifies a node. At downstream nodes where a pC_{DNA} is added to a product a node hash value is computed with the H(C_{DNA}) of the added oligonucleotide. This approach, however, is not favoured as it does not offer the security benefit of adding a pC_{DNA} tag to the product at the earliest possible point in a supply chain.

### Reconstructing Supply Chain Information from Products Labelled with Oligonucleotide Tag Methodology 1 (OTM1) Where Order is Stored in Binary Tree of Hashes

For the genesis hash methodologies 1501-1506, the efficiency with which genesis hash is computed and validated from a product sample is improved by first restricting the database search field to a package identifier that is cryptographically linked to the pC_{DNA} in a product (as disclosed previously). If the product is unpackaged, then genesis hash identification from a product sample alone requires computing all possible H(A) given the pC_{DNA} in a sample, and comparing these values against a database of H(A). The efficiency of computing all H(A) depends on which of the above approaches is taken 1501-1506, but in none of these approaches is computational efficiency prohibitive.

To reconstruct the full tree of identification/custody after a genesis hash is found the order in which the other pC_{DNA} are added in OTM1 must be iteratively reverse engineered. This is achieved by computing all possible node 2 hash values and cross-validating these values against the set of chains that contain the already validated genesis hash. The process of reverse engineering is required in case there are forks in the chain/tree of identification/custody, which may occur when a tagged product ingredient is split and/or recombined to produce two or more different finished products (for example). The probability of a collision between two different combinations of H(C_{DNA}UI) and HC_{DNA}NI) in a product is essentially zero for practical applications.

Methods to link nodes together were disclosed above in Hash Methodologies L1 and L2. Level 1 methodologies (HM_L1) disclosed ways to hash information at each discreet node. Level 2 methodologies (HM_L2) disclosed ways to link hashed information at nodes to form a list or a n-ary tree of hashes.

FIG. 16 shows a diagram 1601 of pC_{DNA} oligo tags sequentially added to a product at three nodes and two methodologies for computing and recording node hash values. The first methodology 1602 is a binary tree of hashes and the second methodology 1603 is a simple hash list. Note that the first methodology 1602 may take a binary or n-ary structure. Other hash structures disclosed above include a Merkle trees.

In first methodology 1602, a hash of each C_{DNA} (and optionally elements of the set X) are sequentially hashed together in a binary tree of hashes, and this information is stored on a distributed, decentralised, or centralised database. In the example 1602 each node hash value is hash of a previous node hash value (a history) concatenated with information about a new node (from set X).

Methodology 1602 permits unpackaged samples to be easily identified by computing different binary permutations of hashes derived from information in a product sample (see Section below and above) until a match is found. This approach presents a number of advantages over a simple list:
A hash chain/tree prevents record tampering, since all hashes are hashed together.
A hash chain/tree allows the order in which oligo tags are added to a product to be recovered.
Unique hash values generated at each node may be used an identifier to append and store other product information (i.e. time stamp, transaction and custody data, quality control information, batch umber, expiry date, manufacturing facility etc.)
The use of H(C_{DNA}) as part of a genesis hash allows full supply chain coverage and improves the security of the transaction of a physical good.

The second methodology 1603 simply stores a list of H(C_{DNA}UI/NI) in a distributed, decentralised, or centralised database. The list of hashes at each node may also be stored in a distributed transaction ledger. The hash records in a distributed blockchain ledger are protected from tampering by established blockchain methods. To find a H(C_{DNA}UI/NI) the transaction list, in each block is crawled. In this sense, methodology 1602 may not be considered a chain or tree.

### The Use of a Binary Tree of Hashes to Store Node Order Information in Combination with OTM1

This section gives a detailed review of implementing the binary tree of hashes methodology in combination with Oligonucleotide Tag Methodology 1, OTM1. It is to be appreciated that this disclosure covers all combinations of OTM1-3 and hashing methodologies disclosed above.

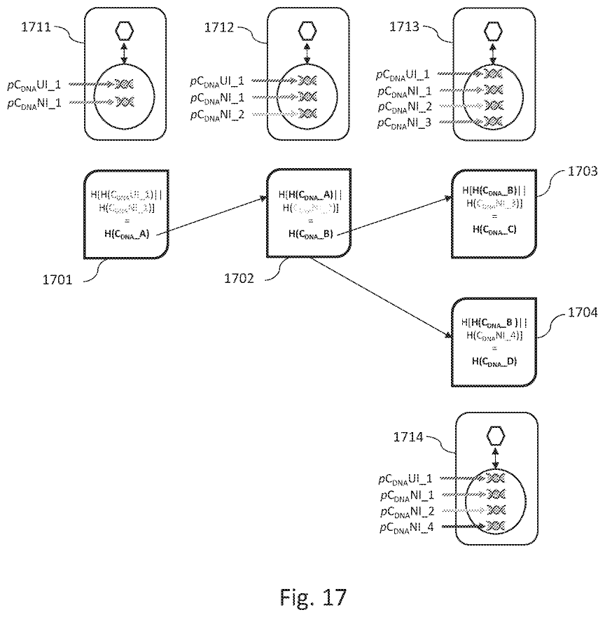

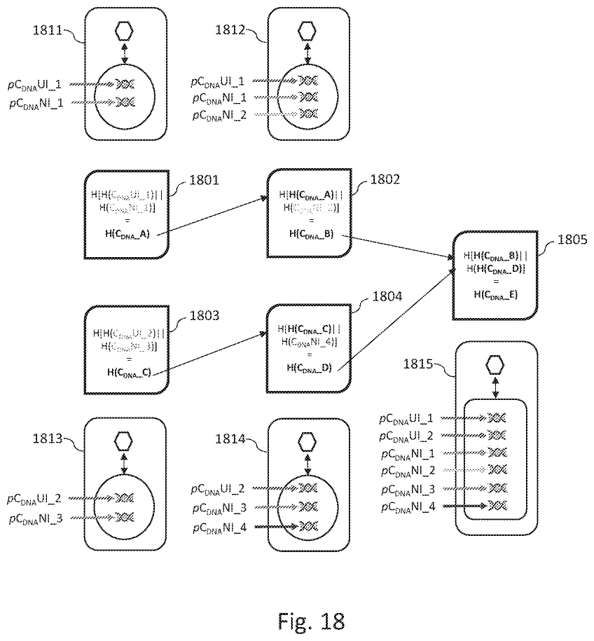

FIG. 17 illustrates the implementation of a fork with binary tree of hashes methodology 1701-1704 and OTM1 1711-1714 and FIG. 18 illustrates how a merge is implemented with binary tree of hashes methodology 1801-1805 and OTM1 1811-1815. For illustrative purposes, these examples only show instances where a pC_{DNA} is added at each node. As descried previously, other identifiers from the set X may also be used.

In FIG. 17 the genesis hash 1701 is a hashed concatenation of two hashed C_{DNA}, H(C_{DNA}_A)=H[H(C_{DNA}UI_1)∥H(C_{DNA}NI_1)]. The physical tags pC_{DNA}UI_1 and pC_{DNA}NI_1 are shown in the packaged product 1711. At node 2 1712 a third tag is added pC_{DNA}NI_2 and the cumulative hash 1702 is computed, H(C_{DNA}_B)=H[H(C_{DNA}_A)| H(C_{DNA}NI_2)]. At this point a fork is performed. In the ‘physical world’ this may occur when a product ingredient is split and sent to two or more different finished product manufacturers (for example). The pC_{DNA} in the product at 1712 are automatically transferred to the split products at 1713 and 1714. The node hash values for products 1713 and 1714 are computed in 1703 and 1704, respectively, and use the same methodology as in 1701 and 1702. Note that in FIG. 17 the pC_{DNA} added at 1713 and 1714 may certify a quality control step or chain custody. In these cases, although the product is the same, the chain of custody is different and therefore the final hash values are different.

In FIG. 18 the hashes at 1801, 1802, 1803, and 1804 are computed using the same binary hash tree methodology as in the fork example in FIG. 17. In FIG. 18, however, a merge is performed between the nodes 1802 and 1804 at node 1805. At the merge point 1805, no pC_{DNA} are added and a ‘virtual’ binary hash is implemented between the node hash values at 1802 and 1804, H(C_{DNA}_E)=H[H(C_{DNA}_B)∥H(C_{DNA}_D)]. Additional pC_{DNA} may be added and hashed downstream from a merge point.

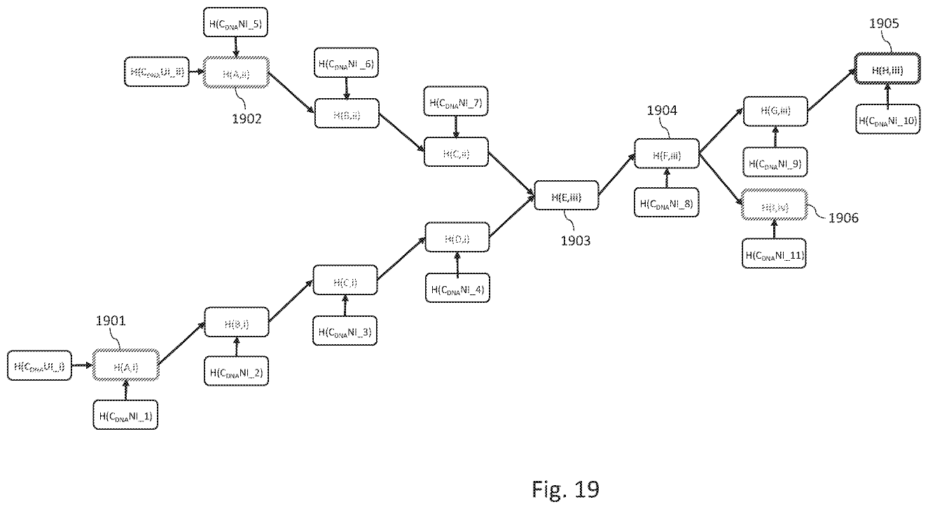

FIG. 19 shows an example of a binary hash tree that includes a merge at node 1903 between the branches 1901 and 1902, and a fork at node 1904. Note that downstream from 1903 and 1904 additional pC_{DNA} are added and recorded in the tree. The final hash values 1905 and 1906 are unique even though both end products share a similar history. Information encoded into pC_{DNA} and added to a product is automatically transferred to new products during fork and merge operations.

### Hashing When No Oligo Tags are Added at a Node

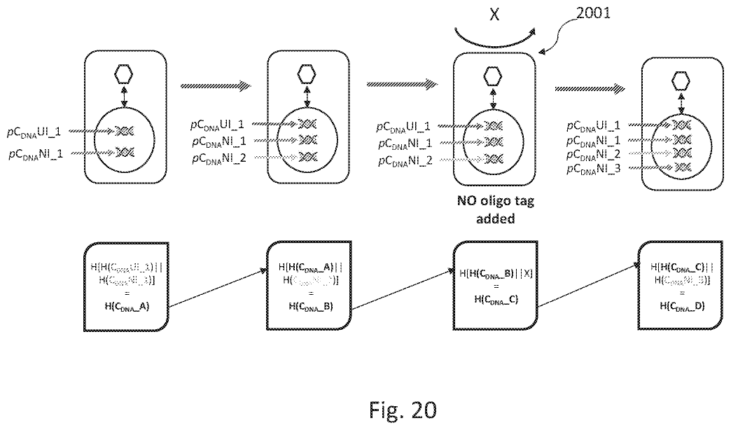

FIG. 20 illustrates how hashing is performed when no oligo pC_{DNA} tags are added to the product. At node 2001 the previous node hash is hashed with one or more of the set X excluding a C_{DNA}, H(C_{DNA}_C)=H[H(C_{DNA}_B)∥X].

If a time stamp or arbitrary counter is used in the operation at 2001, then:
The time/counter interval should be set so that it is sufficiently short to capture a single transaction, but sufficiently long so that a suitable number of hashes is generated over a specified time period to permit decoding.
For example, if the time stamp interval is set to one minute, and assuming a time period of 10 years, 5,256,000 possible node hash values at 2001 may need to be computed and cross-validated to find a valid value for H(C_{DNA}_C). Given a hash mining rate of 330 B hashes s⁻¹, and assuming there are 10 pC_{DNA} in a product, all possible hash values at a timestamped node may be computed<0.0001 seconds.
When a node hash value is validated, then additional information that was appended at the time of the hash value creation (ie. custody information, quality control information, import information, etc.) can be obtained.

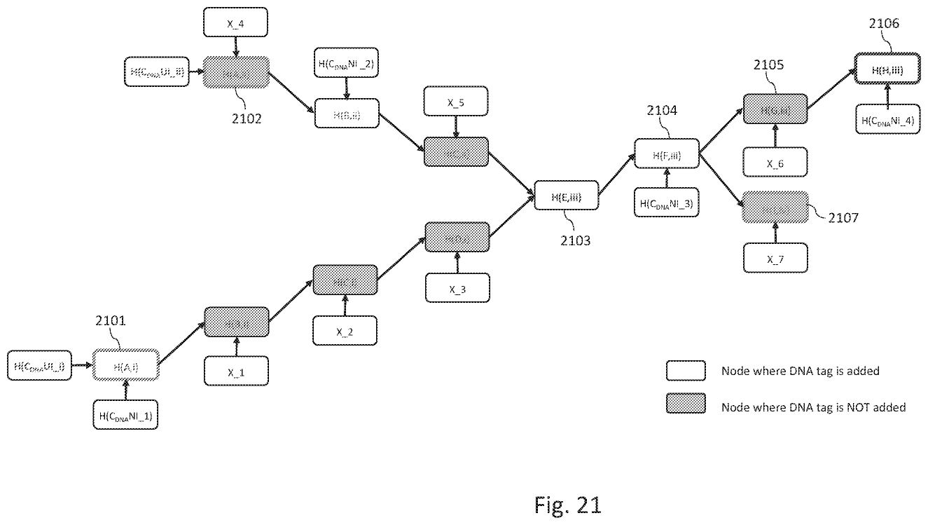

FIG. 21 illustrates a binary tree of hashes with a merge and a fork, and includes node hashes with elements from the set X where no pC_{DNA} tag is added. In this example, the genesis hash of the first chain 2101 is a hashed concatenation of two C_{DNA}, H[H(C_{DNA}UI_i)∥H(C_{DNA}NI_1)]. The genesis hash of the second chain 2102 is a hashed concatenation of one C_{DNA} and X, H[H(C_{DNA}UI_ii)∥H(X_4)]. The two chains merge at 2103 with a virtual hash, H[H(D_i)∥H(C_ii)]. At 2104 a fork is performed and node 2105 and 2107 are both hashed with an element of the set X. If X is a time stamp with an interval of 1 min, a unique hash value will be generated only if operations at 2105 and 2107 are performed >1 min apart. The time interval should be set so that collisions are sufficiently improbable. The final hash values 2106 and 2107 are computed from the combined histories at each node and new information added at 2106 and 2107.

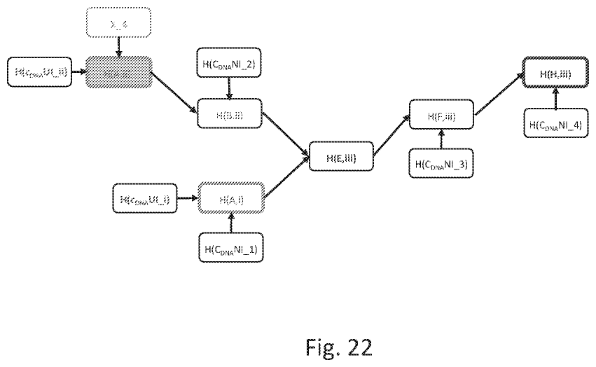

FIG. 22 is a representation of FIG. 21 that only shows nodes where a pC_{DNA} was added. The section below discloses how to reconstruct a full hash chain/tree from the pC_{DNA} detected in an unpackaged product labelled using OTM1 methodology, for e.g. how to reconstruct FIG. 21 from FIG. 22.

### Recovering and Decoding Supply Chain Information from an OTM1 Labelled Product

Here, two main approaches for re-constructing supply chain information from a product sample labelled with OTM1 where order is stored in a binary tree of hashes are disclosed

### Reconstruct Hash Tree Sequentially from the Genesis Hash

Step 1. Find all H(C_{DNA}UI) in a product sample
Step 2. Compute all possible genesis ‘A’ level hashes by iteratively hashing each C_{DNA}UI against each C_{DNA}NI in the sample. The number of possible genesis hash combination is small, n(C_{DNA}UI)×n(C_{DNA}NI).

OR, depending on the method used:

Compute all possible genesis ‘A’ level hashes by iteratively hashing each C_{DNA}UI against all possible values that each element in the set X can take.
Step 3. Compare hash values generated in Step 2 to a database of genesis hash values until a match is found.
Step 4. If the chain stops before all H(C_{DNA}UI/NI) have been account for, try hashing the two chains together - there could have been a ‘virtual’ merge, as shown in FIG. 21.
Step 4. Restrict the search field to ‘B’ level node 2 hashes, in tree associated with the validated genesis hash in Step 3.
Step 5. Compute all possible ‘B’ level hashes using the methodology in Step 2.
Step 6. Compare hash values generated in Step 5 with the restricted node search field in Step 4.
Step 7. Repeat Steps 2-6 until all pC_{DNA} in the sample are accounted for and a terminal hash is found.

If two pC_{DNA} are used to generate the genesis hash and one additional pC_{DNA} is added at each node, the total number of possible hashes c is:

$$c = u \cdot ( ( {n}^{2} ) / 2 )$$

where n is the number of node identifiers in the sample and u is the number of product unique identifiers in the sample.

### Reverse Engineering a Hash Chain/Tree from a Package Identifier Value that is Cryptographically Linked to the pC_{DNA} in Product

This approach may be performed when a package identifier technology includes a node hash value computed at the point of completed product manufacture and packaging:
Step 1. Look up all genesis hashes that are associated with the node hash value displayed on the package identifier technology.
Step 2. Compute all possible genesis ‘A’ level hashes by iteratively hashing each C_{DNA}UI against each C_{DNA}NI in the sample. The number of possible genesis hash combination is small, n(C_{DNA}UI)×n(C_{DNA}NI).

OR, if X is used:

Compute all possible genesis ‘A’ level hashes by iteratively hashing each C_{DNA}UI against all possible values that each element in the set X can take.
Step 3. Compare hash values generated in Step 2 against those in Step 1 until a match is found.
Step 4. If a match is found, iteratively validate all other nodes using the methodology in Step 2 against appropriately restricted search fields.

It is also possible to reverse engineer a chain/tree through brute force from the top-down (ie terminal hash to genesis hash), although this approach is more computationally expensive than those described above. For example, consider the following two scenarios:

Scenario 1. Ten nodes are labelled with pC_{DNA}, and 10 pC_{DNA}NI are detected in a sample This means that the C_{DNA} space is n=10 and the node space is t=10 . In this scenario there are n!−(n−t)!=n!=10!˜ 3.63×10⁶ possible terminal hash values, which is a number that may be easily brute forced. Given a hash computation rate of 330×10⁹ hashes s⁻¹, this would take ˜0.00001 seconds to compute.

Scenario 2. The genesis hash incorporates one pC_{DNA}, t=10 nodes are hashed with a timestamp and there are n=5,256,000 possible time stamp intervals (10 year search field with an time interval of 1 min). In this scenario, there are n!−(n−t)!=1.6×10⁶⁷ hash computations required to cover the terminal hash space. This number is too big to brute force from the ‘top down’. Given a hash computation rate of 330×10⁹ hashes s⁻¹, this would take ˜1.54×10⁴⁸ years, or 1.04×10³⁸ times longer than the universe has existed to generate all possible hash trees.

However, the terminal hash value in this scenario may be brute forced from the ‘bottom up’ (ie. computing and sequentially validating hash values at each node from the genesis hash to the terminal hash). With bottom-up methodology n+(n−1)+(n−2)+. . . +(n−t)˜52.56×10⁶ different hash permutations cover the possible terminal hash space. Given a hash computation rate of 330×10⁹ hashes s⁻¹, this would take ˜0.0002 seconds to compute.

### The Use of a Package Identifier to Display Node Hashes

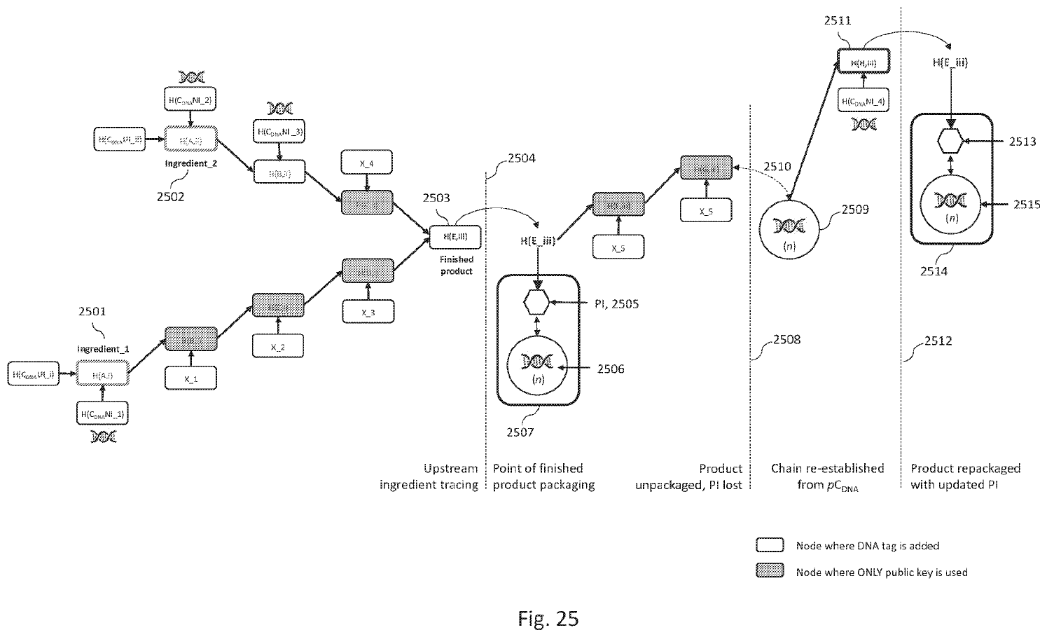

This section discloses methodologies to cryptographically link the pC_{DNA} in a product to a code displayed with a package identifier technology (PI). The PI-C_{DNA} code serves three main purposes: (1) it provides a link between a product and a package, (2) it improves the computational efficiency of reconstructing a hash chain/tree from a product sample through restricting the search field used for cross validating node hashes, and (3) it provides an identifier code that can easily be used to extend a chain of custody/information at downstream nodes where no pC_{DNA} tag is added. With respect to point (3) the identifier code may be used to extend the chain by hashing with elements of the set X. The resulting new virtual nodes may be stored on a distributed, decentralised, or centralised database. This virtual chain extension may be hashed again with a H(C_{DNA}) at any downstream node where a pC_{DNA} is added (as shown in FIG. 25).

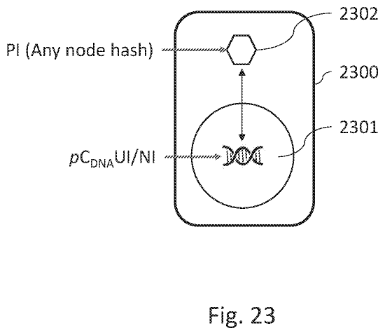

A package identification technology (PI) is any technology that is displayed on a package for the purpose of identifying a product. Package identification technologies may include, but are not limited to: inks, dyes, holograms, bar codes, QR codes, RFID, silicon dioxide encoded particles, product spectral image data, and IoT devices. FIG. 23 shows a packaged product 2300 that is labelled with one or more pC_{DNA}UI/NI 2301 which are cryptographically linked to a package identifier (PI) technology 2302. The PI may display a hash value at any node. The PI code, therefore, incorporates at least one H(C_{DNA}UI/NI) and zero or more elements of the set X.

The use of hashing functions permits a safe and secure link between the pC_{DNA} tags in the product, and the product packaging.
PI is displayed publicly on the package
H(C_{DNA}) provides a cryptographic link to the pC_{DNA}, whilst keeping the C_{DNA} codeword secret.
PI incorporates at least one H(C_{DNA}) of a pC_{DNA} in a product.
The PI code may be a genesis hash, the most recent node hash at packaging, or any other node hash in a product's hash chain/tree.
The PI may be an alternative identifier that points to a node hash value.

### Product Validation for OTM1

As described previously, product validation involves reconstructing a tree of hashes from the pC_{DNA} in a product sample and cross validating this tree against a tree stored in a database. Briefly:
If a product is unpackaged, the hash chain/tree may be reconstructed by brute forcing and sequentially cross-validating permutations of possible node hashes from the genesis node to the most recent terminal node (see above).
If a product is packaged the PI code is first used to restrict the search field to cross validate reconstructed node hashes from pC_{DNA} in a product.

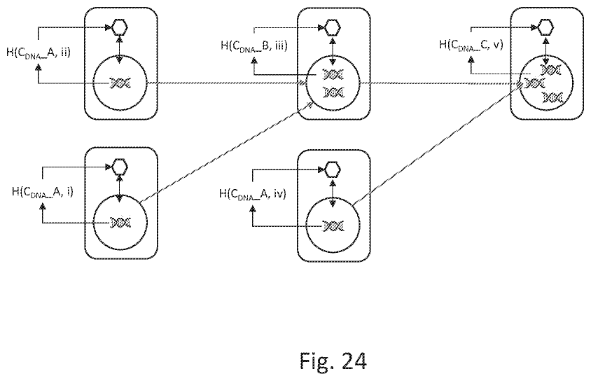

FIG. 24 illustrates a cumulative H(C_{DNA}UI/NI) linked to package identifier technology. The advantage of this approach is that the C_{DNA}UI/NI fragments in the product are explicitly linked to a package marker and can aid in validation. The cumulative node hash value may include elements of the set X as described.

### Repairing a Hash Tree from a Mixed Unpackaged Product

A hash tree may be repaired from a mixed unpackaged product. After a product sample is recovered and decoded, a hash tree may be repaired by hashing the two terminal node hashes together in a ‘virtual’ binary hash. This operation is essentially identical to the merge described in FIG. 18 but should be restricted to persons with the correct permissions (i.e. persons who are authorised to repackage and sell the product).

### Example of a Chain that is Merged, Forked, Broken and Repaired

FIG. 25 is a diagram of an example hash chain/tree that is merged, forked, broken, and then repaired. The chain/tree commences at two different genesis hashes that show a first pC_{DNA} labelled ingredient 2501 and a second pC_{DNA} labelled ingredient 2502. The two ingredients are mixed together to produce a finished product at merge point 2503. Before merging, three operations/transactions are performed on ingredient 2501 which are recorded by hashing with an element of the set X. Two operations are performed on ingredient 2502 which are recorded by hashing with a third H(C_{DNA}) and one element of the set X.

At the merge point, the finished product hash value 2503 is transferred to a package identifier technology 2505 at the point of finished product packaging 2504. The package identifier 2505 is encoded with the hash value at 2503 which is displayed publicly on the package of the oligonucleotide tagged product 2506. In this example, the packaged product 2507 then undergoes two further operations that are recorded by hashing with an element of the set X. These operations may represent custody transactions in a supply chain or a quality control step, for example.

At point 2508 the packaged product 2507 is unpackaged 2509 and the package identifier technology 2505 is lost. The hash tree is reconstructed 2510 from the pC_{DNA} in the unpackaged product 2509 according to methodologies described previously. In this example an additional pC_{DNA} label is added to the unpackaged product to repair the hash chain/tree at node 2511. The product is repackaged at 2512 and a hash value computed at 2511 is transferred to a second package identifier technology 2513. The second package identifier 2513 is displayed on the re-packaged oligo-tagged product 2514, 2515.

### Notes on Security and Reverse Engineering C_{DNA} from H(C_{DNA})

Here, the security of the disclosed invention is investigated from the point of view of an administrator, a sampler and a counterfeiter. The following scenario considers the computational resources required to brute force a hash chain of 10 nodes that are each labelled with one pC_{DNA}.

Administrator. Assume the administrator supplies 1,000,000 pC_{DNA} to customers assume that 10 are added to a product along its supply chain. In this example, therefore, the C_{DNA} codeword space is n=1,000,000 and the node space is t=10. If the administrator knows the cumulative hash value of each node in the chain and tries to brute force the final terminal hash value, the number of hash computations required is: n+(n−1)+(n−2)+. . . +(n−t)=9,999,955. Given a mining rate of 330 B hashes s⁻¹, it would take ˜0.0001 seconds to cover the hash space. If the administrator only knows the final hash value, the number of brute force computations required is: n!−(n−t)!˜1060. Given a mining rate of 330×10⁹ hashes s⁻¹, it would take 9.6×10⁴⁰ years to cover the entire hash space by brute force which is clearly not feasible.

Sampler. The same scenario is now considered from the sampler's perspective (or more accurately sampling software's perspective). The sampler obtains the hash value of each of the 10 pC_{DNA} in the product but does not know the order in which the tags were added. The sampler, therefore, must derive this order by comparing the hash of each combination of H(C_{DNA}) obtained from a product. In this example the codeword space n=10 and the node space t=10. If the sampler knows the cumulative hash values at each node then the number of final node hash values that need to be brute forced to cover the hash space is: n+(n−1)+(n−2)+. . . +(n−t)=55. This number can easily be brute forced. It would take 1.1×10⁻¹⁰ seconds. If the sampler only knows the final hash value of the chain the number of hashes that need to be computed to cover the space of all final hash values is n!−(n−t)!=10!=3,628,800. This number can also be easily brute forced. It would take 1.1×10⁻⁵ seconds.

Counterfeiter: The same scenario is now considered from the counterfeiter's perspective. Assume that a counterfeiter does not have any knowledge about the pC_{DNA} supplied, and does not know the encoding system used. This means the counterfeiter has to test all combinations of possible Z₄ encoded oligonucleotide fragments. For the purpose of this exercise, assume the counterfeiter knows the encoding region of a fragment is 60 nucleotides long and that 10 fragments have been added to a product. Here, the possible C_{DNA} fragment codeword space is n=4⁶⁰=1.33×10³⁶ and the node space is t=10. If the counterfeiter knows the cumulative hash values at each node, then the space of possible final hash values is: n+(n−1)+(n−2)+. . . +(n−t)=1.33×10³⁷. Given a mining rate of 330×10⁹ hashes s⁻¹, it would take 1.40×10¹⁸ years to compute all possible final node hashes (or ˜97×10⁶ times longer than the universe has existed). Similarly, if the counterfeiter only knows the final node hash, the number of computations required to cover all possibilities is n!−(n−t)!=(1.33×10³⁶)!−(1.33×10³⁶−10)!˜1.33×10³⁴¹ years. It is therefore infeasible for a counterfeiter to reverse engineer the C_{DNA} codes in a product by brute force.

The scenarios above show that the proposed system is vistually impossible to hack, but may be used by an authorized person with the right permissions.

### Storing Hash(DNA) Data in a Block Chain

Here, a brief review of block chain technology is given and then a description of different approaches to storing H(C_{DNA}) in blockchain architecture is discussed.

### Overview of Blockchain—Key Processes

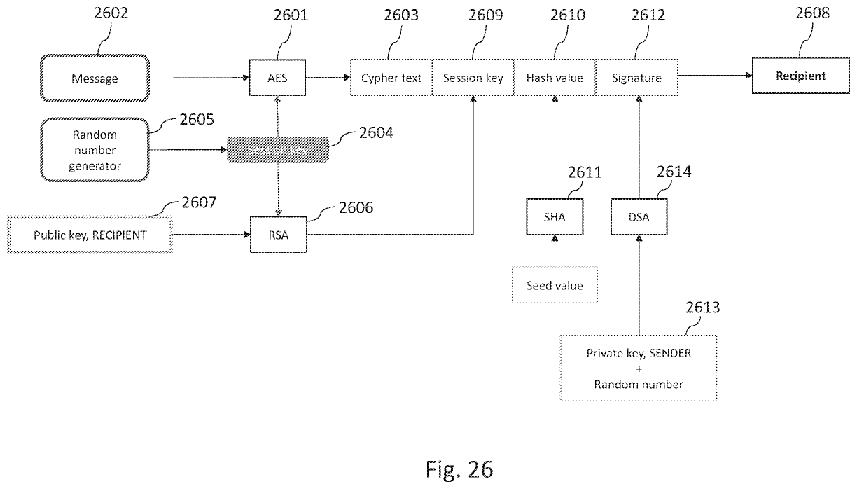

FIG. 26 illustrates a public key encryption protocol used transfer information between two parties, where the transaction may be recorded on a distributed ledger and protected by blockchain. In FIG. 26 AES 2601 is an Advanced Encryption Algorithm that is used to convert plain message text 2602 into cypher text 2603. In the disclosed invention H(C_{DNA}) information may be stored inside the plain message text. The AES 2601 uses a session key 2604 that is generated by a random number generator 2605 or trusted key infrastructure. An RSA (Rivest, Shamir, Adleman) 2606 algorithm uses a public key 2607 of a recipient 2608 to encrypt a session key 2609, which is appended to a cyphertext 2603.

The appended Cypher text 2603 and Session key 2609 are then hashed to give a Hash value 2610 of the Cypher text 2603 plus Session key 2609 block. The hash 2601 may be calculated by SHA, secure hash algorithm, 2611 or similar. The Hash value 2610 is unique to a particular Cypher text 2603 plus Session key block 2609 in the sense that a single bit change in those inputs radically change the hash 2610 and is used to ensure that the data are not modified by a hacker.

A Sender (not shown) then signs the entire block by providing a signature 2612, which is based on the Sender's private key and a random number 2613 encrypted with a signature algorithm 2614 such as DSA (digital signature algorithm). On the recipient side, these four algorithms are carried out in reverse to get the original plain text message. First the sender's signature is used to verify the sender. Then the receiver checks the hash value of the message.

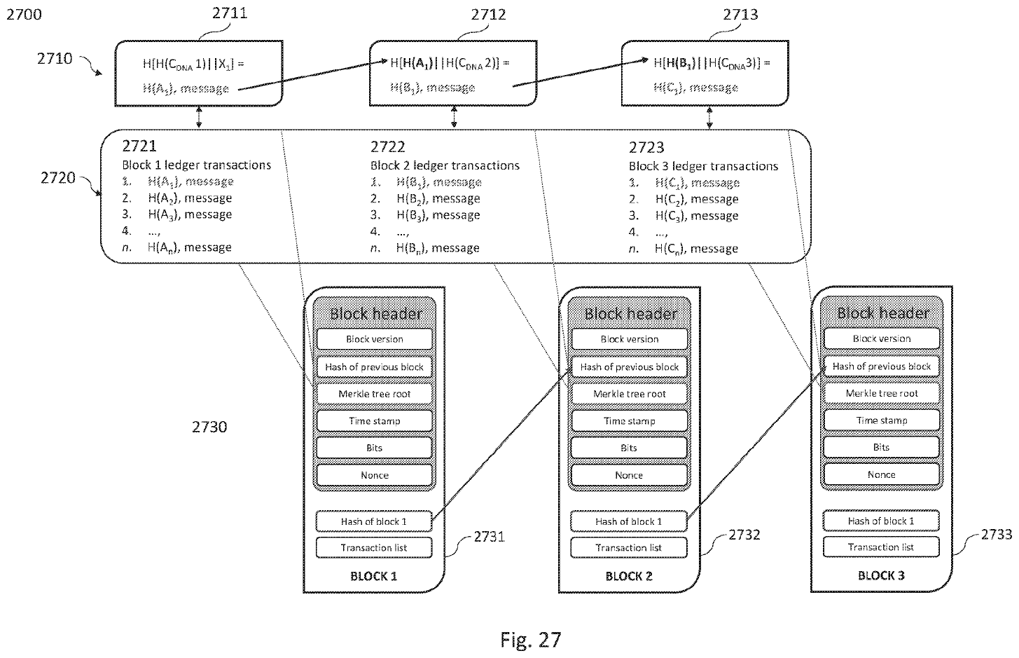

FIG. 27 illustrates a system for product tracing and verification where supply chain information is stored in physical oligonucleotide tags that are integrated into a product and backed up on an immutable blockchain. In FIG. 27 H(C_{DNA}) information is transacted between the digital wallets of members 2710, stored in a distributed ledger 2720, and protected by blockchain architecture 2730. When at transaction between two members occurs, a node hash value derived from one or more H(C_{DNA}) is computed and used as an identifier for associated message information. The node hash and associated message information is stored in a distributed ledger 2720. The data stored in a distributed ledger is processed in blocks that are protected by a blockchain. In the example, the transactions between wallets 2711, 2712, 2713 are stored in a different ledger block 2721, 2722, 2723 although this does not need to be the case.

In this example, a block in a block chain 2730 is comprised of:
The Block header is 80 bytes
The Block version (4 bytes) specifies the software version and is changed when software is upgraded.
The Hash previous block (32 bytes) is a hash of the previous block header and is update when a new block is set
The Hash of Merkle root (32 bytes) is a binary hash tree of all of the transactions stored in a block, and is updated until new transactions stop being added.
The Timestamp (4 bytes), and is updated every few seconds
The Bits (4 bytes) are used to set the difficulty of mining a block and are updated when the mining difficulty needs to be adjusted
The Nonce (4 bytes) is a number only used once, whose value is such that the hash of the block contains a run of leading 0's.

Consensus on each block hash value is achieved between participants through a process called mining. A block is ‘mined’ when a nonce value is found such that Hash(Hash block header (including the nonce))=hash with a defined number of 0's. The number of 0's sets the difficulty. Typically, the nonce value is located on the left most leaf of a Merkle tree representation of a block data in a distributed ledger. Any change in the nonce value will result in a change in the Merkle root value.

Mining is the process of iteratively trying different nonce values, and testing these values against a generated Merkle root value. When a miner finds a solution such that Merkle root value =a string that contains a pre-defined leading run of 0's, the miner advertises their solution to the network. Other members in the network check the solution, and if verified, the block is added to the block chain. A hash of the mined block is then passed to the next block. In this way, each block 3031, 3032, 3033 is connected together in an immutable chain.

### Key Advantages of the Disclosed Invention

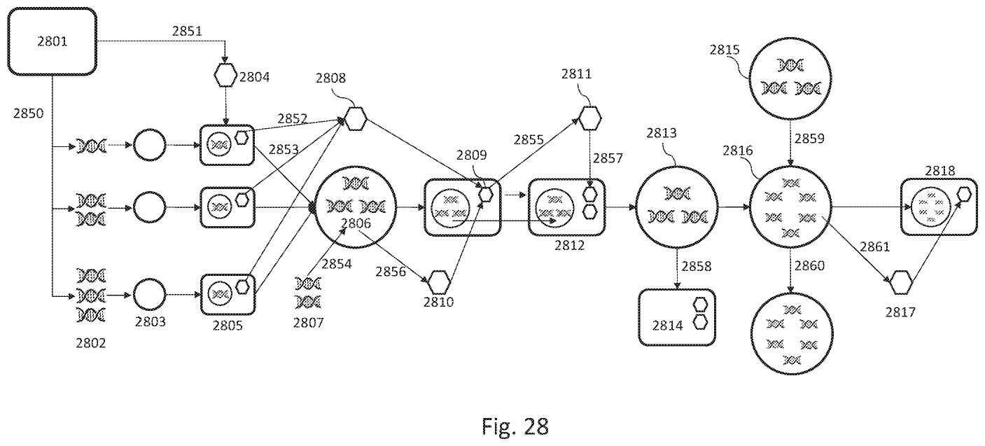

FIG. 28 illustrates key information transfers between one or more oligonucleotide labelled products that are mixed, unpackaged, split, and repackaged.

A unique identifier is encoded into an oligonucleotide tag that is added to an item. The unique identifier may be optionally linked one or more package technologies that are attached to the item downstream in the supply chain. The unique identifier may be recovered from either the oligonucleotide tag or the package technology. Information associated with the unique identifier may be stored on a distributed ledger, decentralised database, or centralised database. The key advantages of the proposed oligo tag—blockchain system is that (1) the oligo tags are product integrated and protected by a molecular ‘lock and key’ which makes counterfeiting virtually impossible, (2) the oligo tags secure the supply chain upstream from the point of finished product manufacture, and downstream from the point of unpackaging. (3) the oligo tags are ‘automatically’ transferred upon mixing which permits full traceability of composite goods, and (4) that chain of supply/provenance may be re-established if an item is unpackaged or the package identifier technology is damaged (for example). In FIG. 28 there is shown an oligonucleotide encoder 2801 that encodes one or more oligonucleotides 2802 with unique identification information using the set of four base pairs, {A, C, G, T} and possibly U. There are also one or more product ingredients 2803 labelled with the one or more oligonucleotide tags. A package unique identifier technology (inks, dyes, barcodes, IoT device, etc) 2804 contains information linked to oligonucleotide tag/s in the product. One or more package devices 2805 are optionally attached to packaged oligo-labelled ingredients. Oligo-labelled ingredients are recombined into a final product 2806, containing multiple oligo-labelled ingredients. One or more oligonucleotide tags 2807 may optionally be added at the point of completed product manufacture. A package unique identifier technology 2808 may be linked to oligo tag/s in a completed product (QR codes, barcodes, IoT, etc) using information from package identifier technologies linked to oligo-tags in a product's ingredients.

A packaged finished product 2809 with oligo-integrated tag/s is linked to package identifier technology 2810 that is attached to finished product packaging 2809. There may be a second, third or more ‘layered’ package identification or security device (e.g. IoT device) 2811 and a packaged finished oligo-labelled product 2812 with one or more package identifier technologies 2811 attached to it.

FIG. 28 then shows an unpackaged finished product 2813 and a discarded finished product packaging 2814 (package identification and security technologies also discarded) as well as a second, third or more oligo-tagged finished product 2815 tagged with one or more oligos encoded with unique identifiers. There is also a second finished product 2816 comprised of one or more recombined finished products containing oligo-labels encoded with one or more unique identifiers.

Accordingly, there may be one or more unique package identifier/s 2817 with information recovered from oligo tag/s in product 2816 and a recombined, repackaged, product 2818 with chain of provenance restored from the oligo tags in the product.

The following description provides a method for verifying a product's identity including information transfers between different entities and modules. Unique identifier/s are encoded 2850 into oligonucleotide fragment/s and mixed/labelled into ingredients. A unique identifier in 2801 is encoded 2851 into one or more package technologies 2804 attached to ingredient package 2805. Information from unique package identifier/s in 2805 is transferred 2852 to a second package technology attached to a finished product package. Additional information may be added to a package unique identifier in 2808. Additional information optionally encoded into another unique oligo identifier/s 2854 and added to the finished product 2806. Information from unique oligo identifiers in 2806 is optionally transferred 2856 to package unique identifier 2810 (2nd route). One or more additional package technologies (ie. barcode, QR code, IoT, etc.) are optionally attached 2857 to/included in finished product packaging. Information from package technologies is discarded 2858 upon unpackaging. Information from one or more different finished products is transferred 2859 via oligo-tag to a new re-combined finished product 2816. If a new recombined product is split 2860 the information in the pC_{DNA} tags is transferred. A chain of provenance is restored 2861 from the oligo-tag/s in an unpackaged recombined product, and this information is incorporated into a new package unique identifier technology 2817 that is displayed on a repackaged product 2818.

### Oligo-Tag Sample Preparation, Encoding and Decoding

This section gives a background of oligo nucleotide encoding, oligonucleotide decoding, and sample preparation noting that error detection and correction code may be employed by the systems and methods of this disclosure. This is because even a single nucleotide error in any oligonucleotide fragment in a product may result in a hash value error that propagates to all downstream nodes in a hash tree. This type of error may render product validation from the pC_{DNA} tags in a product impossible. Errors mostly occur during oligonucleotide synthesis or oligonucleotide sequencing.

Error detection and correction code is particularly important for the compatibility of the disclosed technology with Oxford Nanopore technology. Ocfor nanopore offers portability and low read latency, but has a significantly higher sequencing error rate compared to other platforms (˜10% for short fragments).

### Oligonucleotide Sample Preparation

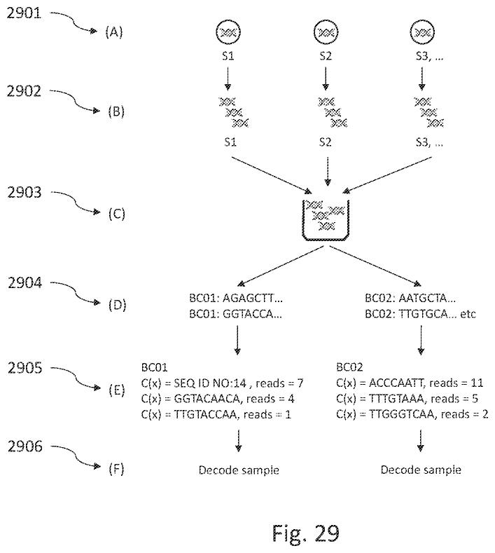

FIG. 29 illustrates oligonucleotide tag sampling and sample preparation.

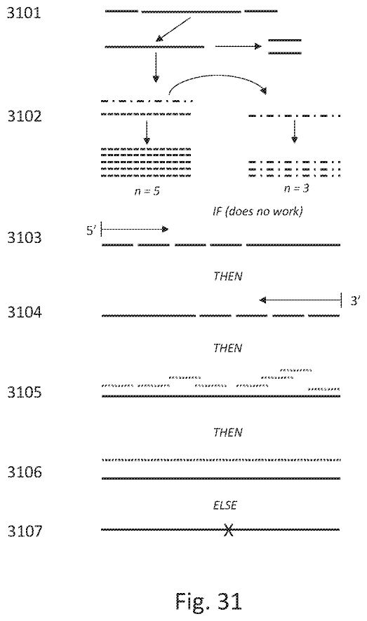

In 2901 samples of products are shown that contain one or more oligonucleotide tags each. The oligo tags are encoded with a unique identifier. The samples are amplified 2902 with primers comprised of a site that is complementary to a primer site in the target sequence, and a barcode sequence (BC) that identifies a sample. This may involve locked nucleic acids (LNA) as described in PCT/AU2017/050757 filed on 21 Jul. 2017 and entitled “A METHOD FOR AMPLIFICATION OF NUCLEIC ACID SEQUENCES”. The amplified and barcoded samples are pooled together 2903 and prepared for sequencing according to standard protocols, and then sequenced. The sequenced fragments are partitioned 2904 according to their respective barcode sequence that identifies the sample. Each sample may optionally be further partitioned into similar sets of codewords 2905 based on a semi-global sequence alignment with the strands previously sequenced in the sample and the read count recorded. The base-called data for each sample are then decoded 2906 (See FIG. 31).

### Oligonucleotide Error Detecting and Correcting Encoding Approaches

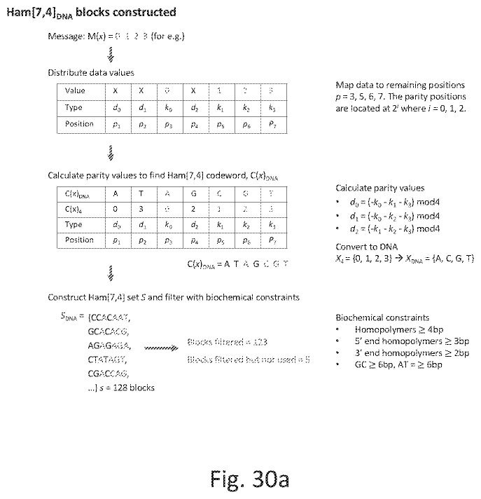

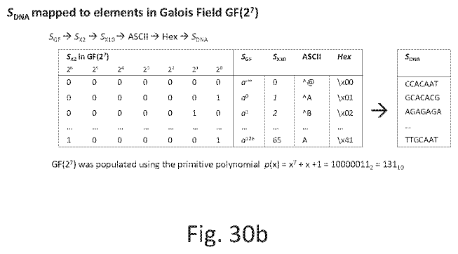

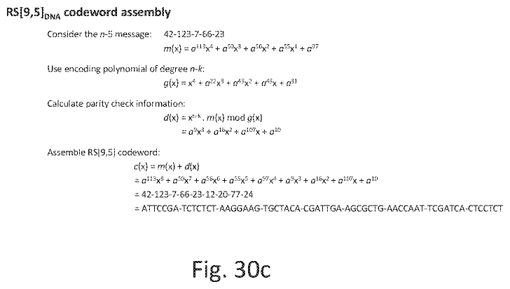

FIGS. 30a, 30b and 30c illustrate an example of an oligonucleotide encoding system optimized for nanopore sequencing. As previously explained, the set of nucleotides Sₙ={A, C, G, T} is of size sₙ=4. In FIG. 30a a set of DNA symbols is encoded in Z₄ using Hamming Ham[nᵢ, kᵢ] code, where the symbol length is nᵢ=7 is comprised of kᵢ=4 data nucleotides and dᵢ=nᵢ−kᵢ=3 parity nucleotides. This design ensures that each block is separated by a mutual minimum distance of dₘᵢₙ=3 nucleotides. Ham[7,4] also permits error detection of 2 b (bases or nucleotides) and error correction of 1b per symbol. The size of the set of possible Ham[7,4] blocks is sₛ=sₙ^{ki}=256 symbols. After filtering for biochemical constraints, the set of possible symbols was reduced to 133 symbols. This number of symbols was sufficient to cover the elements of a Galois Field GF(2⁷) =GF(128) used to encode Reed Solomon (RS) codewords. The size of the set of Ham[7,4] symbols S_{DNA} needed to encode a RS codeword in GF(128) is S_{DNA} (or sₛ)=128 symbols.

In some instances a terminal sequence may be added to each symbol in S_{DNA}. This approach aids decoding in circumstances where large insertion and deletion errors result in a catastrophic frameshift error that cannot be decoded by conventional Hamming and Reed-Solomon decoding approaches.

In FIG. 30b the set of Ham[7,4] symbols S_{DNA} is mapped to the elements in a GF(128). The example in FIG. 30c shows standard procedures to encode a Reed Solomon codeword. In this example, RS[9,5] code is used where n=9 symbols, comprised of k=5 data symbols and d=n−k=4 parity symbols. This system permits burst error detection and correction capability of d/2=2 symbols or 14 nucleotides, and a codeword library size of >34 billion codewords. This approach was found to be compatible with Oxford Nanopore technology given the error rate and type of this device.

It should be appreciate that any combination of Ham[n, k] and RS[n, k] inner or outer codeword combinations may be used. The example in FIG. 30a,b,c shows a RS[9,5]-Ham[7,4] design.

### Oligo Tag Decoding Algorithm

FIG. 31 illustrates a methodology for oligonucleotide decoding that comprises the following steps:

First, base-called data are partitioned into samples according to the barcode sequence attached via PCR ligation at sample recovery. Primer site sequences are used to detect complementary strands which are optionally converted into equivalent template strands. The primer sites are then cleaved off 3101 to obtain a query sequence codeword, qC_{DNA}. A set of qC_{DNA} in each sample may optionally be partitioned into codeword sets 3102 based on the similarity of a qC_{DNA} to previously partitioned and decoded qC_{DNA} in a sample. This step involves full fragment length semi-global sequence alignment. In 3103 codeword query sequences are first string split from 5′ end 3103 into blocks of symbol length n nucleotides. A string split sequence is decoded by first correcting symbols using Hamming decoding approaches and then applying RS decoding procedures. This approach is likely to be successful if symbols towards the 3′ end of a fragment are un-decodable with Hamming methodology due to insertion and deletion errors. If decoding is unsuccessful, then a query sequence is string split from the 3′ end 3104 into blocks of symbol length n nucleotides. A string split sequence is decoded by first correcting symbols using Hamming decoding approaches and then applying RS decoding procedures. This approach is likely to be successful if symbols towards the 5′ end of a fragment are un-decodable with Hamming methodology due to insertion and deletion errors. If step 3104 is unsuccessful local sequence alignment is optionally performed 3105 against the set of symbol sequences used to encode the fragment. The best alignment for at least n−d/2 symbols is found and then standard RS decoding is performed. If n−d/2 symbols do not meet a defined alignment threshold, then full fragment length semi-global sequence alignment analysis 3106 against previously decoded sequences in the sample, or all codeword sequences in a database of issued codewords, may optionally be performed. If a defined threshold is not met with full fragment length semi-global sequence alignment, then a query sequence is discarded 3107.

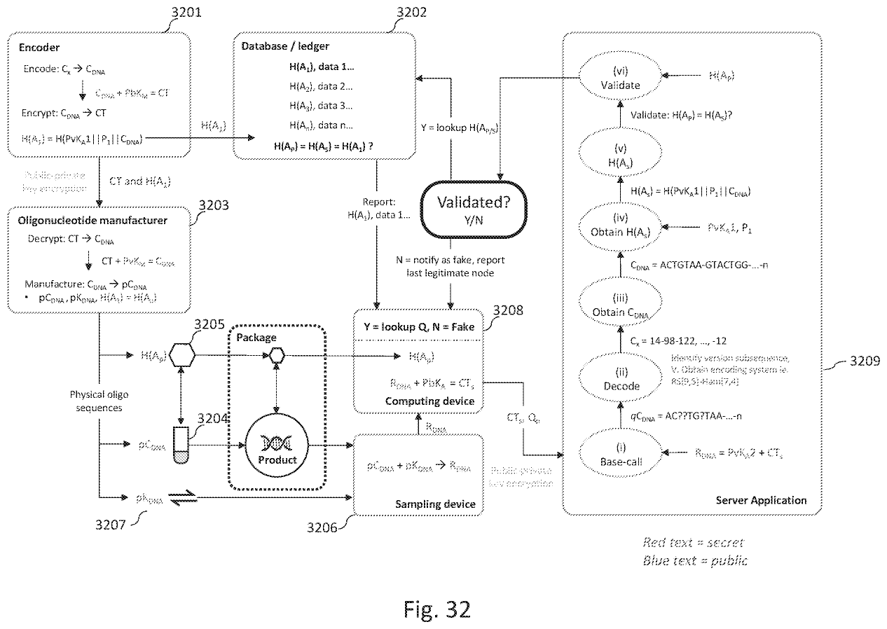

FIG. 32 is another example that shows how a codeword is encoded into an oligonucleotide 3201, encrypted, hashed, sent to a database 3202 (distributed, decentralised or centralised), manufactured 3203, added to a product 3204, included in a package identifier technology 3205, sampled from a product using a sampling device 3206 and oligonucleotide keys 3207 in combination with a local computing device with a sequencing application 3208, decoded from a product sample with an application on a server 3209 and validated against a database of hash values 3202.

The symbols in FIG. 32 include:
PbK_{A}: Public key administrator (public)
PvK_{A}1: Private key administrator 1 (secret)
PvK_{A}2: Private key administrator 2 (secret)
PbK_{M}: Public key manufacturer (public)
PvK_{M}: Private key manufacturer (secret)
PvK_{S}: Private key sampler (secret)
CT: ciphertext (public)
H(A₁): A hash that includes C_{DNA} (public)
H(A_{P}): A package identifier code that is H(A₁) (public)
H(A_{S}): A hash value that is derived from pC_{DNA} in a sample (public)
Cₓ=Alphanumeric message text (secret)
C_{DNA}=an oligonucleotide codeword (secret)
pK_{DNA}=Physical oligonucleotide key (secret)
pC_{DNA}=Physical oligonucleotide fragment encoded with codeword (secret)
qC_{DNA}=Query oligonucleotide codeword (secret)
P₁=padding text 1 (public)
P₂=padding text 2 (public)
H( )=hash function (public)
∥=concatenated text
R_{DNA}=Raw oligonucleotide sequence data, not base-called (secret)

In FIG. 32 there is shown an encoder 3210 that encodes a codeword into an oligonucleotide sequence, calculates a hash value of the codeword and stores the hash value in a database 3202. In this example the hash value is a concatenation of an administrators private key, padding text, and an oligonucleotide codeword, H(PvK_{A}1∥P₁∥C_{DNA}) although many other variants are possible. A manufacturing machine 3203 synthesises a physical oligonucleotide sequence 3204 that is added to a product and a hash value of the sequence is incorporated into a package identifier technology and displayed on a package 3205. A sampling device 3206 is used to recover an oligonucleotide sequence/s from a product using an oligonucleotide key sequence 3207, and provides raw sequence data to a computing device 3208. The steps performed by the computing device 3208 are provided in a method as described above.

The raw data is encrypted by the computing device 3208 and set to an application on a server 3209. The server application base-calls the raw data, decodes the base-called sequence/s to derive corrected oligonucleotide codeword/s, calculates query hash value/s for the corrected codeword/s and compares the query hash value/s against hash values stored in database 3202. In this example note that padding text and an administrators private key is applied to calculate sample hash values. In other words, the computing device uses a product identifier as a look-up key in the database to retrieve the correct/expected hash for that product. If the hashes match, the product's identity is verified. This may also be referred to as product authentication.

The following description provides further information on the decoding steps. In particular, the sequencing on some platforms may comprises a significant amount of errors that lead to a misalignment with the codewords and code symbols of the code. Therefore, computing device 214 may perform an alignment step to align the sequenced oligonucleotide sequence from the product against a stored oligonucleotide sequence. Then, the computing device 214 can calculate the hash value based on the aligned nucleotide sequence in the sense that the computing device 214 uses the aligned sequence in the decoding step and then calculates the hash after decoding. The alignment step provides a further mechanism to increase the robustness of the system. In particular, the alignment step is useful where individual bases or parts of the sequence have been deleted.

In cases where the oligonucleotide sequence is generated using multiple code symbols, such as the Hamming symbols described above, computing device 213 can align the sequenced second oligonucleotide sequence against the multiple code symbols. Further, where generating the oligonucleotide sequence is based on generated codewords, such as the RS codewords described above, computing device 214 can align the sequenced second oligonucleotide sequence against previously decoded codewords or a database of codewords.

With these different options available, it is possible to selectively choose one of the alignment options. This may be based on a sequencing error so that the alignment is performed against multiple code symbols for relatively low error rates as the computational complexity for code symbol alignment is relatively low. As an alternative, on in addition, the alignment can be performed against multiple codewords for relatively high error rates as the computational complexity for this codeword alignment is relatively high.

The following description provides further details starting again from the encoding steps for DNA fragment encoding.

The relatively high error rate of ON technology required sufficient redundancy for reliable decoding. This section describes the RS[9,5]-Ham[7,4] encoding system used to reliably recover information from the encoded DNA fragments.

### Hamming Encoded DNA Symbols

Codeword symbols were constructed with Hamming[nᵢ, kᵢ, dᵢ] code, where nᵢ is the block length in nucleotides, kᵢ is the number of data nucleotides, and dᵢ is the number of parity nucleotides (1, 2). The minimum Hamming distance between symbols is also given by dᵢ and the rate is given by r=kᵢ/nᵢ. Herein we use the shorthand specification Ham[nᵢ, kᵢ], where dᵢ=nᵢ−kᵢ. In this example we used Ham[7,4] blocks. The inner symbol code (denoted by subscript i) specification used to generate the Ham[7,4] blocks, was:

nᵢ=7, is the total number of nucleotides
kᵢ=4, is the number of ‘data’ nucleotides
dᵢ=nᵢ−kᵢ=3, is the number of parity nucleotides
dₘᵢₙ=dᵢ=3, is the minimum distance between each block
rᵢ=kᵢ/nᵢ=0.571=1.14 bits b⁻¹ is the rate, or data density of a symbol

As defined by Hamming code, parity (dᵢ) nucleotides were located every 2ⁿⁱ positions in the quaternary symbol (Table 1). In the case of the Ham[7,4] code the parity nucleotides d₀, d₁, d₂ are located at positions 1, 2, 4 and the data nucleotides, k₀, k₁, k₂, k₃ at positions 3, 5, 6, 7. Symbols were constructed by mapping the quaternary set of nucleotides Qₙ={A, C, G, T} of size sₙ=4 to the quaternary numeral set Q₄={0, 1, 2, 3} and binary set Q₂={00, 01, 10, 11}.

In Table 1 the parity nucleotides cover the positions marked ‘x’, such that the encoded block satisfies:

$$( {d}_{0} + {k}_{0} + {k}_{1} + {k}_{3} ) mod 4 = 0$$

$$( {d}_{1} + {k}_{0} + {k}_{2} + {k}_{3} ) mod 4 = 0$$

$$( {d}_{2} + {k}_{1} + {k}_{2} + {k}_{3} ) mod 4 = 0$$

$$( {d}_{0} + {d}_{1} + {k}_{0} + {d}_{2} + {k}_{1} + {k}_{2} + {k}_{3} ) mod 4 = 0 ( included for Ham [ 8 , 4 ] )$$

The value of the parity nucleotides was calculated by:

$${d}_{0} = ( - {k}_{0} - {k}_{1} - {k}_{3} ) mod 4$$

$${d}_{1} = ( - {k}_{0} - {k}_{2} - {k}_{3} ) mod 4$$

$${d}_{2} = ( - {k}_{1} - {k}_{2} - {k}_{3} ) mod 4$$

$${d}_{3} = ( {d}_{0} + {d}_{1} + {k}_{0} + {d}_{2} + {k}_{1} + {k}_{2} + {k}_{3} ) mod 4 ( included for Ham [ 8 , 4 ] )$$

The size of the set of Ham[7,4] symbols in the library Sₛ is sₛ=4⁴=256. Each symbol in Sₛ (S_{DNA} is Sₛ throughout) is separated by a minimum mutual distance of d=3 b (b is base or nucleotide). The full set of Ham[7,4] symbols in Sₛ is given in Table 2.

To final set of symbols was obtained by filtering the candidate set of 256 Ham[7,4] symbols with biochemical constraints to avoid GC-rich and homopolymer sub-regions upon codeword assembly. The following constraints eliminated homopolymer sub-sequences>4b in a codeword:
Internal homopolymers≥4b
5′ end homopolymers≥3b
3′ end homopolymers≥2b
AT and GC content≥6b
3b GC at 5′ or 3′ end
Internal GC sequence of ≥5b

These constraints filtered out 123 symbol sequences leaving 133 candidate symbols which was sufficient to cover the 128 elements in Galois Field GF(2⁷). Five symbols passed biochemical filtering but were not needed and discarded.

Reed-Solomon Codeword Assembly, RS[9,5]-Ham[7,4]

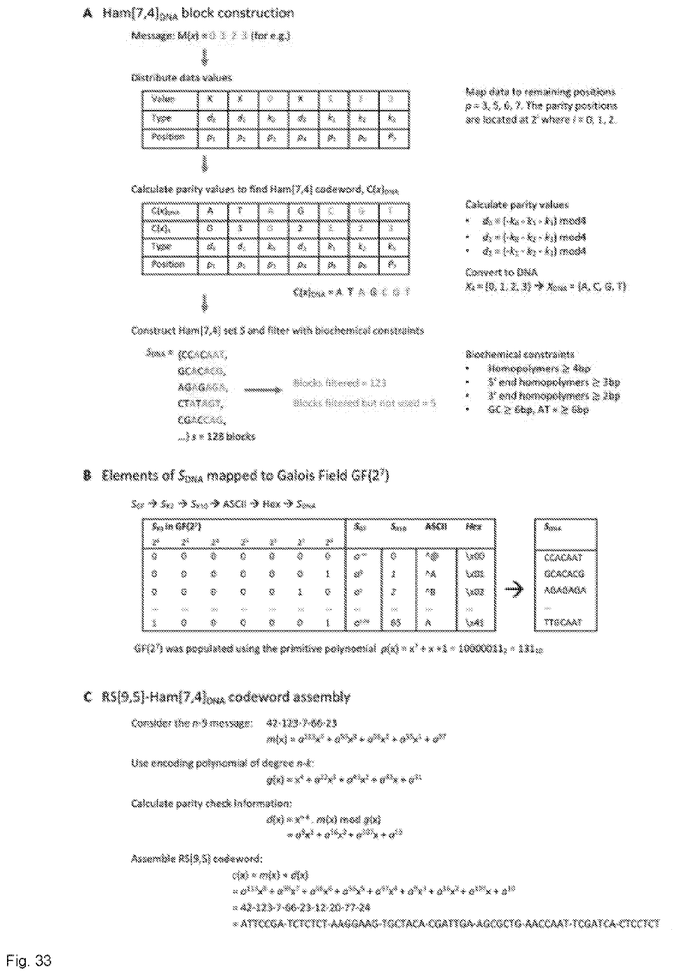

Table 2 and FIG. 33 show how Reed-Solomon codewords, c(x) were constructed. Codewords were assembled by mapping the set of 128 Ham[7,4] blocks to the set of 128 symbols of degree m=7 over Galois Field GF(2^{m})=GF(2⁷)=GF(128). Symbols in GF(128) were generated using the irreducible polynomial p(x)=x⁷+x+1=100000112=13110, setting the first element to a⁰=0x^{m-1}+0x^{m-2}+. . . +1x+0=x, and recursively multiplying a. The element values were taken as the binary m-tuple vector coefficient of each element polynomial generated by p(x) according to Galois Field theory, and labelled with GF(128) symbols {a^{-∞}, a⁰, a¹, . . . , a¹²⁶}. The full set of GF(128) elements and Ham[7,4] DNA encoded blocks is given in Table 2.

The full specification of the Reed-Solomon codewords used was RS[n, k] 2t, where:
n=the number of Ham [7,4] symbols in the codeword=9,
k=the number of message symbols in the codeword=5,
n−k=the number of parity check symbols=4
t=(n−k)/2=the forward error detection and correction capability=2 symbols
nᵢ=number of nucleotides in each symbol=7

The RS[9,5] codewords c(x) contained five message symbols m(x) and four parity check symbols d(x). This design permitted a codeword space of w=s_{GF}^{k}=1285>34 billion unique codewords. Parity check information d(x) was obtained from Equation S1 according to Reed-Solomon theory:

$$c ( x ) = m ( x ) + d ( x ) \qquad Equation S1$$

$$d ( x ) = {x}^{n - k} \cdot m ( x ) \cdot mod g ( x )$$

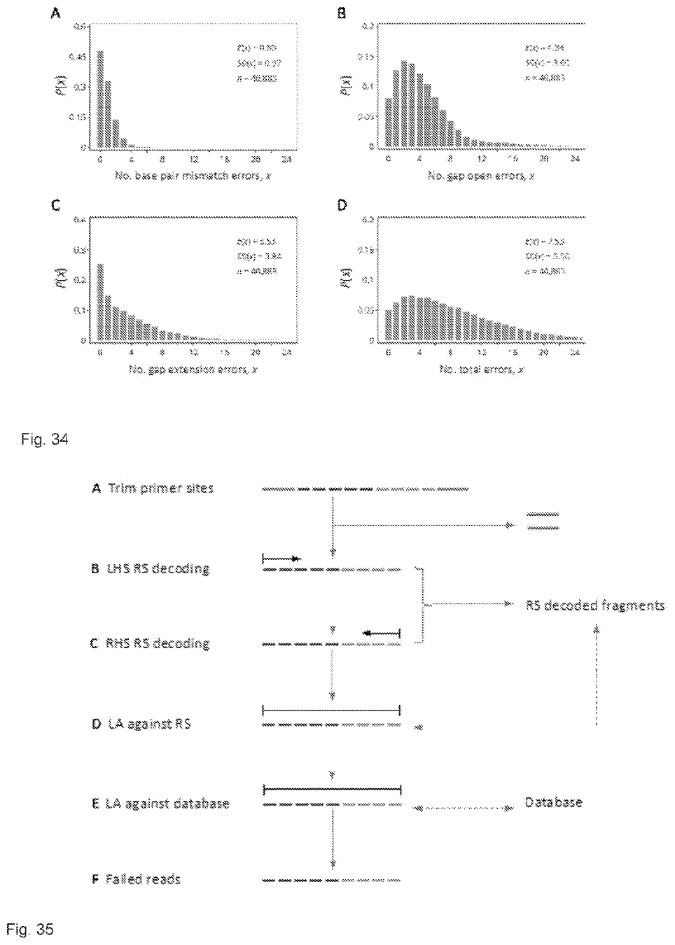

Although the density of our RS[9,5]-Ham[7,4] encoding system is 0.63 bits b⁻¹, significantly less than the theoretical maximum of 2 bits b⁻¹, this design allowed us to detect and correct 2t=4, t=2 symbol errors or burst errors of ≤14 nucleotides. This level of redundancy was required given the relatively high error rate of ON technology for short fragment length sequencing (See FIG. 34).

The sequence and design specifications of the fragments used in the experiments are given in Table 6.

TABLE 1
Ham[7,4] encoding system.
Ham[7,4] encoding design

| | Position in n | | | | | | |
|---|---|---|---|---|---|---|---|
| | 1 | 2 | 3 | 4 | 5 | 6 | 7 |
| Type | d₀ | d₁ | k₀ | d₂ | k₁ | k₂ | K₃ |
| d₀ | x | | x | | x | | x |
| d₁ | | x | x | | | x | x |
| d₂ | | | | x | x | x | x |
| d₃ | x | x | x | x | x | x | x |
| (for Ham[8,4]) | | | | | | | |

In this table, k₀₋₃ are data bits and d₀₋₂ are the parity bits. The ‘x’ marks the positions covered by the parity nucleotides.

TABLE 2
The alphabet set of GF(128) elements mapped to Ham(7,4) DNA symbols

| S_{GF} | X₂ | X₁₀ | ASCII | Hex | S_{DNA} (Ss) | S_{GF} | X₂ | X₁₀ | ASCII | Hex | S_{DNA} (Ss) |
|---|---|---|---|---|---|---|---|---|---|---|---|
| a⁻∞ | 0000000 | 0 | ^{∧@} | \x00 | CCACAAT | a⁶³ | 0001001 | 9 | ^{∧}I | \x09 | GTATATG |
| a⁰ | 0000001 | 1 | ^{∧}A | \x01 | GCACACG | A⁶⁴ | 0010010 | 18 | ^{∧}R | \x12 | GACCAGC |
| a¹ | 0000010 | 2 | ^{∧}B | \x02 | AGAGAGA | a⁶⁵ | 0100100 | 36 | $ | \x24 | GACTGAT |
| a² | 0000100 | 4 | ^{∧}D | \x04 | CGACCAG | a⁶⁶ | 1001000 | 72 | H | \x48 | ATCGGTC |
| a³ | 0001000 | 8 | ^{∧}H | \x08 | TCACAGC | a⁶⁷ | 0010011 | 19 | ^{∧}S | \x13 | TCGCGAC |
| a⁴ | 0010000 | 16 | ^{∧}P | \x10 | TTAGCCA | a⁶⁸ | 0100110 | 38 | & | \x26 | TCGGCTG |
| a⁵ | 0100000 | 32 | [space] | \x20 | TGACCGA | a⁶⁹ | 1001100 | 76 | L | \x4c | AATGCCA |
| a⁶ | 1000000 | 64 | > | \x3e | ACAACAT | a⁷⁰ | 0011011 | 27 | ^{∧}[ | \x1b | CGTCATA |
| a⁷ | 0000011 | 3 | ^{∧}C | \x03 | CTATAGT | a⁷¹ | 0110110 | 54 | 6 | \x36 | GAGAGAG |
| a⁸ | 0000110 | 6 | ^{∧}F | \x06 | GTAATGT | a⁷² | 1101100 | 108 | 1 | \x6c | CTGACTA |
| a⁹ | 0001100 | 12 | ^{∧}L | \x0c | AGCGCTG | a⁷³ | 1011011 | 91 | [ | \x5b | TCAACTA |
| a¹⁰ | 0011000 | 24 | ^{∧}X | \x18 | CTCCTCT | a⁷⁴ | 0110101 | 53 | 5 | \x35 | CGTACAT |
| a¹¹ | 0110000 | 48 | 0 | \x30 | ACAGTGC | a⁷⁵ | 1101010 | 106 | j | \x6a | AGTTGAT |
| a¹² | 1100000 | 96 | ′ | \x60 | CACCACG | a⁷⁶ | 1010111 | 87 | W | \x57 | AGGCTCT |
| a¹³ | 1000011 | 67 | C | \x43 | ACGTATG | a⁷⁷ | 0101101 | 45 | - | \x2d | CTGTGAT |
| a¹⁴ | 0000101 | 5 | ^{∧}E | \x05 | CTACGAC | a⁷⁸ | 1011010 | 90 | Z | \x5a | GTGCATA |
| a¹⁵ | 0001010 | 10 | ^{∧}J | \x0a | ATAGCGT | a⁷⁹ | 0110111 | 55 | 7 | \x37 | CCAGTTA |
| a¹⁶ | 0010100 | 20 | ^{∧}T | \x14 | AACCAAT | a⁸⁰ | 1101110 | 110 | n | \x6e | TCCAGAG |
| a¹⁷ | 0101000 | 40 | ( | \x28 | AGCATCA | a⁸¹ | 1011111 | 95 | _ | \x5f | AAGAGGA |
| a¹⁸ | 1010000 | 80 | P | \x50 | TGCCGTG | a⁸² | 0111101 | 61 | = | \x3d | GATCGAC |
| a¹⁹ | 0100011 | 35 | # | \x23 | CTGGTCG | a⁸³ | 1111010 | 122 | z | \x7a | GGTGTTA |
| a²⁰ | 1000110 | 70 | F | \x46 | CAGCCGA | a⁸⁴ | 1110111 | 119 | w | \x77 | GGTCAAT |
| a²¹ | 0001111 | 15 | ^{∧}O | \xof | ACACATA | a⁸⁵ | 1101101 | 109 | m | \x6d | TGATTAG |
| a²² | 0011110 | 30 | ^{∧∧} | \xle | GGCTAAC | a⁸⁶ | 1011001 | 89 | Y | \x59 | CTTGAGA |
| a²³ | 0111100 | 60 | < | \x3c | TGCATAC | a⁸⁷ | 0110001 | 49 | l | \x31 | TACGTGC |
| a²⁴ | 1111000 | 120 | x | \x78 | AACGTTA | a⁸⁸ | 1100010 | 98 | b | \x62 | CAAGGTC |
| a²⁵ | 1110011 | 115 | s | \x73 | ATGTGTA | a⁸⁹ | 1000111 | 71 | G | \x47 | CTAATCA |
| a²⁶ | 1100101 | 101 | e | \x65 | GACGTCG | a⁹⁰ | 0001101 | 13 | ^{∧}M | \x0d | TGTCACG |
| a²⁷ | 1001001 | 73 | I | \x49 | CATGCGT | a⁹¹ | 0011010 | 26 | ^{∧}Z | \xla | TATCGCA |
| a²⁸ | 0010001 | 17 | ^{∧}Q | \x11 | TAAGGCT | a⁹² | 0110100 | 52 | 4 | \x34 | AATCGGT |
| a²⁹ | 0100010 | 34 | ″ | \x22 | TACACAT | a⁹³ | 1101000 | 104 | h | \x68 | TTGGTTA |
| a³⁰ | 1000100 | 68 | D | \x44 | GTACGCA | a⁹⁴ | 1010011 | 83 | S | \x53 | ACCTTGA |
| a³¹ | 0001011 | 11 | ^{∧}K | \x0b | ATACGTG | a⁹⁵ | 0100101 | 37 | % | \x25 | ACTAATC |
| a³² | 0010110 | 22 | ^{∧}V | \x16 | CTAGCTG | a⁹⁶ | 1001010 | 74 | J | \x4a | GTGGTGC |
| a³³ | 0101100 | 44 | , | \x2c | AGCTAGT | a⁹⁷ | 0010111 | 23 | ^{∧}W | \x17 | CGATTGA |
| a³⁴ | 1011000 | 88 | X | \x58 | AGCCGAC | a⁹⁸ | 0101110 | 46 | . | \x2e | TCCGAGA |
| a³⁵ | 0110011 | 51 | 3 | \x33 | GATATCA | a⁹⁹ | 1011100 | 92 | \ | \x5c | GCAGTAT |
| a³⁶ | 1100110 | 102 | f | \x66 | TGTTGTA | a¹⁰⁰ | 0111011 | 59 | ; | \x3b | TCAGTCG |
| a³⁷ | 1001111 | 79 | O | \x4f | TAGTTGA | a¹⁰¹ | 1110110 | 118 | v | \x76 | CTCTCTC |
| a³⁸ | 0011101 | 29 | ^{∧}] | \xld | AGAAGAG | a¹⁰² | 1101111 | 111 | o | \x6f | GATTAGT |
| a³⁹ | 0111010 | 58 | : | \x3a | GAATCTC | a¹⁰³ | 1011101 | 93 | ] | \x5d | GTCGGCT |
| a⁴⁰ | 1110100 | 116 | t | \x74 | GTCAATC | a¹⁰⁴ | 0111001 | 57 | 9 | \x39 | GTAGCAC |
| a⁴¹ | 1101011 | 107 | k | \x6b | TATGCAC | a¹⁰⁵ | 1110010 | 114 | r | \x72 | TACCATA |
| a⁴² | 1010101 | 85 | U | \x55 | GCTGGTC | a¹⁰⁶ | 1100111 | 103 | g | \x67 | GATGCTG |
| a⁴³ | 0101001 | 41 | ) | \x29 | ACTGGCT | a¹⁰⁷ | 1001101 | 77 | M | \x4d | TCGATCA |
| a⁴⁴ | 1010010 | 82 | R | \x52 | CATTACA | a¹⁰⁸ | 0011001 | 25 | ^{∧}Y | \x19 | CATCGTG |
| a⁴⁵ | 0100111 | 39 | ′ | \x27 | GAACTCT | a¹⁰⁹ | 0110010 | 50 | 2 | \x32 | AGTGTCG |
| a⁴⁶ | 1001110 | 78 | N | \x4e | TTCTCCT | a¹¹⁰ | 1100100 | 100 | d | \x64 | ACGATGT |
| a⁴⁷ | 0011111 | 31 | ^{∧}_ | \x1f | GAGGAGA | a¹¹¹ | 1001011 | 75 | K | \x4b | CACTGTA |
| a⁴⁸ | 0111110 | 62 | > | \x3e | AGTCAGC | a¹¹² | 0010101 | 21 | ^{∧}U | \x15 | TCGTAGT |
| a⁴⁹ | 1111100 | 124 | \| | \x7c | CATATAC | a¹¹³ | 0101010 | 42 | * | \x2a | ATTCCGA |
| a⁵⁰ | 1111011 | 123 | { | \x7b | TCTCTCT | a¹¹⁴ | 1010100 | 84 | T | \x54 | TGTGTAT |
| a⁵¹ | 1110101 | 117 | u | \x75 | ATGGTAT | a¹¹⁵ | 0101011 | 43 | + | \x2b | CAGTTAG |
| a⁵² | 1101001 | 105 | i | \x69 | CACGTAT | a¹¹⁶ | 1010110 | 86 | V | \x56 | AGTACTA |
| a⁵³ | 1010001 | 81 | Q | \x51 | TCTTCTC | a¹¹⁷ | 0101111 | 47 | / | \x2f | AGGTCTC |
| a⁵⁴ | 0100001 | 33 | ! | \x21 | GCATGTA | a¹¹⁸ | 1011110 | 94 | ^{∧} | \x5e | GTTCCAG |
| a⁵⁵ | 1000010 | 66 | B | \x42 | TGCTACA | a¹¹⁹ | 0111111 | 63 | ? | \x3f | CTGCAGC |
| a⁵⁶ | 0000111 | 7 | ^{∧}G | \x07 | AAGGAAG | a¹²⁰ | 1111110 | 126 | ~ | ix7e | GCTAACT |
| a⁵⁷ | 0001110 | 14 | ^{∧}N | \x0e | TGGAACT | a¹²¹ | 1111111 | 127 | | \x7f | TTGCAAT |
| a⁵⁸ | 0011100 | 28 | ^{∧}\ | \x1c | ACGGCAC | a¹²² | 1111101 | 125 | } | \x7d | CTTAGAG |
| a⁵⁹ | 0111000 | 56 | 8 | \x38 | ATGCACG | a¹²³ | 1111001 | 121 | y | \x79 | CGTGTGC |
| a⁶⁰ | 1110000 | 112 | p | \x70 | GTGACAT | a¹²⁴ | 1110001 | 113 | q | \x71 | TTACGGT |
| a⁶¹ | 1100011 | 99 | c | \x63 | GACACTA | a¹²⁵ | 1100001 | 97 | a | \x61 | TAGCCAG |
| a⁶² | 1000101 | 69 | E | \x45 | ATCAACT | a¹²⁶ | 1000001 | 65 | A | \x41 | TCATGAT |

### Decoding Steps

### DNA Fragment Decoding

The relatively high error rate of ON technology required sufficient redundancy for reliable decoding. For example, the RS[9,5]-Ham[7,4] system developed has a density of 0.63 bit b⁻¹ (where b is base or nucleotide) which is significantly lower than the maximum of 2 bits b⁻¹. An analysis of DNA sequencing error is given in FIG. 35. Across all RS[9,5]-Ham[7,4] records (n=24,487) and Ham[8,4] records (n =16,396) the expected total error rate was E(x)±SD(x)=7.53±5.56 b. ˜7.5±5.5% (weighted for length). No errors of any type P(x=0) were detected in only 5.1% of reads. The query fragment length was 92-107 b. including forward and reverse primer sites of 22 b each.

The expected error for base mismatches was E(x)±SD(x)=0.80±0.97 b=0.79±0.96%. The expected gap open and gap extension error was 4.34±3.60=4.29±3.56% and 3.53±3.84=3.50±3.80%, respectively. These analyses do not include oligonucleotide synthesis error which may contribute 1%.

### RS[9.5]-Ham[7.4] Fragment Decoding

Due to the relatively high error rate of ON technology, the decoding system developed used a combination of RS decoding, local symbol sequence alignment, and full fragment length sequence alignment. Symbol local sequence alignment compares the similarity of a codeword subsequence against the set symbol sequences used to construct the codeword (S_{DNA} in Table 2). Full fragment length sequence alignment compares the similarity of a codeword against either the set of previously decoded codewords in a sample, or against a database of codeword sequences. In all cases, the Smith-Waterman algorithm for local sequence alignment was used¹ from the software package BioPython2².

The steps described here are illustrated in FIG. 34 and FIG. 35. Note that steps A-C in FIG. 35 are first performed on all query sequences in a sample, step D is performed against the set of sequences successfully decoded in A-C, and step E is performed against a database of fragments.

### A. Primer Trimming

DNA codewords were isolated by trimming nucleotide upstream and downstream of the forward and reverse primer site in a query sequence. Primer site sequences were identified by string searching for the n=7 primer site nucleotides that directly flank a codeword. If no matches were found, the search was reperformed with the corresponding n=7 forward and reverse primer site nucleotides of the complementary strand. If no primer sites were detected, the query sequence was forwarded to step B regardless.

### B. Left-Hand Side Reed-Solomon (LHS RS) Decoding

LHS RS decoding was performed with a sliding window of length nᵢ=7 symbol nucleotides (ie. Ham[7,4]) from the 5′ end (left) of the fragment as shown in FIG. 35. LHS RS decoding is likely to be more successful than RHS RS decoding if there is a higher density of errors at the RHS of the query sequence.

The following steps were taken to decode symbol sub-sequences in a query sequence, and are illustrated in FIG. 35:
i. First, a match score, f, was initialised to 0 for each query sequence.
ii. If a Hamming codeword was detected without error (E=0) in a sliding window, the score was updated according to f=fₚ+7pₘ where fₚ is the previous score and pₘ the base pair match alignment parameter.
iii. If the distance of the window substring was dₕ=1 from a valid hamming symbol (0<E<(nᵢ−kᵢ)/2), the symbol was repaired and the score updated by f=fₚ+6pₘ, where fₚ is the previous score. For both cases (i, ii) the sliding window was subsequently moved forward by nᵢ=7 b.
iv. If the distance of the window substring was dₕ=2 ((nᵢ−kᵢ)/2<E<(nᵢ−kᵢ)/2+1) from a valid hamming symbol, the window was expanded by 1 (nᵢ=8), and local sequence alignment against the alphabet of all Ham[7,4] symbols was performed using the alignment parameters specified below (vi). If the alignment score was >5pₘ the score was updated by f=fₚ+5pₘ. The sliding window was then moved forward by i+1 where i is the index of the last matching nucleotide, and its size was reset.
v. If no matches were found, the sliding window was moved forward by 2 b, and steps (i-iv) repeated.
vi. The alignment parameters used were: pₘ (base pair match)=5.0, pₘₘ (base pair mismatch)=−4.5, p_{go} (gap open)=−2.5, p_{ge} (gap extension)=−2.0. Note that if (nᵢ−kᵢ)/2 is a non-integer value, it is rounded down to the nearest integer.

The following steps were taken to decode codewords from a string of query symbols found in (i-v) above:
i. If the number of symbols detected was less than n−(d/2)=7, the query codeword is not RS decodable, and was forwarded to D.
ii. If 7 symbols were detected, 2 erasure symbols were added so that the query codeword was n=9 symbols long. The n-9 query codeword was converted into an integer polynomial according to the GF(128) element field in Table 2 and RS decoding was performed to repair errors.
iii. If 8 symbols were detected 1 erasure symbol was added so that the query codeword was n=9 symbols long. The n-9 query codeword was converted into an integer polynomial according to the GF(128) element field in Table 2 and RS decoding was performed to repair errors.
iv. A parity check was then performed to ensure the codeword is a valid Reed-Solomon polynomial.
v. Steps (i-iv) were repeated for the complement of the query sequence, and the sequence with the maximum of the two scores was kept.
vi. If no valid Reed-Solomon polynomials were found, the query sequence was forwarded to C.

### C. Right-Hand Side Reed-Solomon (RHS RS) Decoding

The steps of RHS RS decoding are similar to LHS RS decoding. In RHS-RS decoding the sliding window was started at the opposite end of the query sequence and moved from right to left (opposite to that shown in FIG. 2.3). As such the first symbol detected is the last element of the RS polynomial. RHS RS decoding is likely to be successful if there are a higher density of errors at the 3′ end (LHS) of the query sequence.
i. If RHS RS decoding failed, LHS RS decoding was performed according to the steps in B (with relevant adjustments made for a 3′→5′ sliding window).
ii. If LHS RS decoding failed, the query sequence was forwarded to D.

### D. Local Sequence Alignment Against Previously Decoded Fragments (from B, D)

For query sequences not decoded in steps B and C, local sequence alignment was performed against the pool of successfully decoded sequences in B and C.
i. If the alignment score was f>0.7fₘₐₓ (>220), the query sequence was accepted.
ii. If the alignment score was f<0.7fₘₐₓ (<220), the query sequence forwarded to E.

The local sequence alignment parameters used were: pₘ (base pair match)=5.0, pₘₘ (base pair mismatch)=−4.5, p_{go} (gap open)=−2.5, p_{ge} (gap extension)=−2.0. Local sequence alignment was performed with the BioPython package Pairwise2^{2.}

### E. Local Sequence Alignment Against Database Fragments

If a query sequence was not successfully decoded in B, C and D, local sequence alignment was performed as in D against a database of issued fragments.
i. If the alignment score was f>0.7fₘₐₓ (>220), the query sequence was accepted.
ii. If the alignment score was f<0.7fₘₐₓ (<220), the query sequence was rejected.

The same sequence alignment parameters were used as in D.

### DNA Sequencing Error

FIG. 34 shows an nanopore DNA sequencing error analysis. The probability distribution of different error types for RS[9,5]-Ham[7,4] (n=40,883 query sequences) including: (A) base-pair mismatch, (B) gap open, (C) gap extension and (D) total errors. The total expected error rate for the 92-107 b. encoding region was E(x)±SD(x)=7.53±5.56 b˜7.5±5.5% (weighted for length). No errors of any type P(x=0) were detected in only 5.1% of reads. The relatively high error rate of the Oxford Nanopore platform for short read lengths (<120 b) required and encoding with sufficient redundancy for successful decoding (>50%).

### Decoding Steps

FIG. 35 illustrates decoding steps. This figure is a diagram of the decoding steps described above. Briefly, decoding steps include (A) primer site trimming, (B) left-hand side Reed Solomon (LHS RS) decoding, (C) right-hand side Reed Solomon (RHS RS) decoding, (D) local sequence alignment (LA) against successfully Reed-Solomon decoded fragments, (E) LA against a database of issued sequences, and (F) failed reads. Steps B-F are hierarchical in that if B is not successful then C is tried and so on. The fraction of query sequences decoded at each step are given in Table 3.

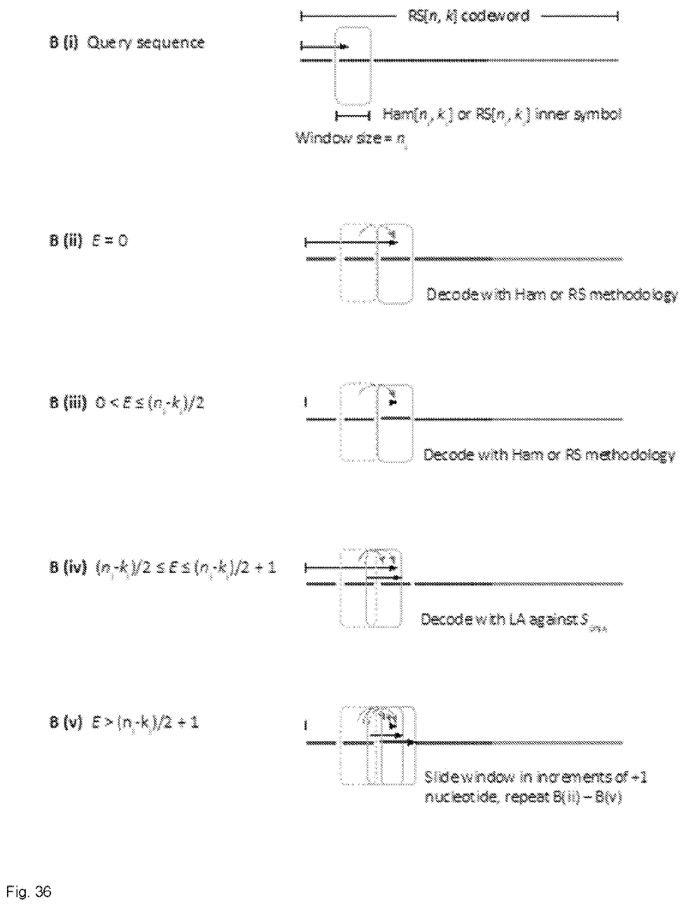

FIG. 36 illustrates the decoding steps graphically particular: B (i) shows a RS codeword comprised of either Ham[nᵢ, kᵢ] or RS [nᵢ, kᵢ] symbols. Decoding is performed with a sliding window of size nᵢ. If the number of errors (E) detected in the window, E=0 B (ii) standard Ham or RS decoding is performed (depending on how the symbol is encoded). If 0≤E≤(nᵢ−kᵢ)/2 B (iii) standard Ham or RS decoding is performed. If (nᵢ−kᵢ)/2≤(nᵢ−kᵢ)/2+1 B (iv) local alignment (LA) against the set of DNA symbols S_{DNA} is performed. If E≤(nᵢ−kᵢ)/2+1 B (v) the sliding window is moved forward by +1 nucleotide and steps B(ii)-B(v) are repeated. When a symbol is successfully decoded, the sliding window is moved forward by i+1, where i is the index of the last matching nucleotide. Not that if (nᵢ−kᵢ)/2 is a non-integer value, the value is rounded down to the nearest integer. Although LHS decoding is shown, these steps also apply to RHS decoding, where the sliding window is moved from right to left as described in the text.

### Analysis

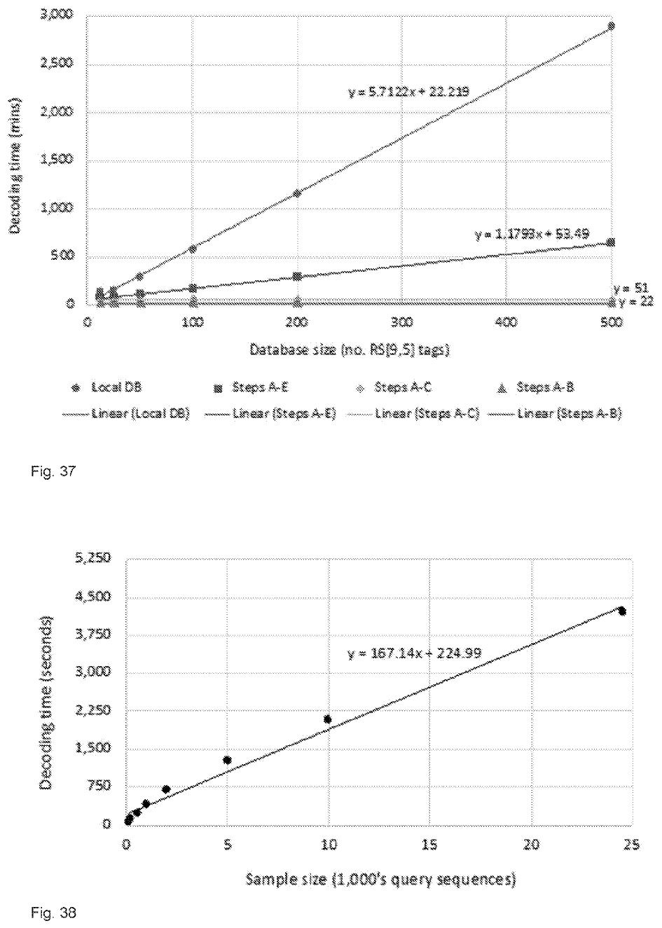

In this section an analysis of the decoding algorithm disclosed above is given for a sample containing 24,487 query sequences. FIG. 37 and Table 3 shows the total number of sequences decoded at each step (A-E) of the decoding algorithm disclosed herein. The RS steps (A and B) decoded a total 18.71% of the query sequences. A further 53.93% of query sequences were decoded at Step C, which compared query sequences to sequences that were successfully decoded in Steps A and B. The decoding efficiency in sequences per second (seqs s⁻¹) was 3.47 and 5.81 seqs s⁻¹ for RS decoding (Steps A and B) and Step C respectively. The final Step D, local sequence alignment against a database of sequences, decoded an additional 10.57% of sequences but resulted in a ˜10-fold reduction in decoding efficiency to 0.53 seqs s⁻¹. In total, 16.79% of query sequences were not decodable. For comparison, if only full fragment length local alignment against a database is used, the decoding efficiency is reduced further to 0.14 seqs s⁻¹.

FIG. 37 is a graphical representation of the data in Table 4 and shows that the decoding tie for Steps A-C, which successfully decoded 72.64% of query sequences was independent of database size. For Steps A-E and for local alignment against a database only, the relationship between decoding time and database size increased linearly, which is not suitable for scaling for use in practical applications. Lastly, FIG. 38 and Table 5 show that the decoding time for Steps A-C vary linearly with sample size, with an average decoding efficiency of 5.81seqs s⁻¹.

The sequence and design specifications of the fragments used in the sequencing experiments are given in Table 6.

### Decoding Results

TABLE 3
Fraction of query sequences decoded at each step in
the decoding algorithm and decoding efficiency.

| | | | Decoding |
|---|---|---|---|
| | Total | Cumulative total | efficiency |
| | (%) | (%) | (seqs s−1) |
| A. LHS RS | 13.31 | 13.31 | NA |
| B. RHS RS | 5.4 | 18.71 | 3.47 |
| C. LA against RS | 53.93 | 72.64 | 5.81 |
| D. LA against DB | 10.57 | 83.20 | 0.53 |
| E. Failed reads | 16.79 | 100 | NA |
| Local against DB | 100 | 100 | 0.14 |

Data show the number of query sequences successfully decoded at each step (n=24,487 query sequences) and the decoding efficiency in seqs s⁻¹. Note that Steps A-C are independent of the database size. Acronyms include: left hand side Reed Solomon (LHS RS), right hand side Reed Solomon (RHS RS), local alignment (LA) and database (DB).

FIG. 37 illustrates an analysis of decoding time against database size. This figure is a graphical representation of the data given in Table 4. Data show the time taken to decode query sequences in a sample (n=24,487 query sequences) as a function of database size. Decoding by local sequence alignment only varied linearly with database size, with an average decoding time efficiency of 0.14 seqs s⁻¹. Steps A-E also varied linearly with database size but with an average decoding time efficiency of 0.53 seqs s⁻¹. The decoding time for steps A-B and A-C was independent of database size with efficiency of 3.47 seqs s⁻¹ and 5.81 seqs s^{-1,} respectively. These data show that codeword length local sequence alignment against sequences successfully decoded by RS significantly increase the number of sequences decoded and the decoding time efficiency. Alignment against a database scales linearly and is not suitable for practical applications.

TABLE 4
Analysis of decoding time against database size.

Decoding time (min)

| DB size | 12 | 25 | 50 | 100 | 200 | 500 |
|---|---|---|---|---|---|---|
| Local DB | 142 | 145 | 294 | 579 | 1153 | 2887 |
| Steps A-E | 79 | 80 | 109 | 169 | 284 | 646 |
| Steps A-C | 51 | 51 | 51 | 51 | 51 | 51 |
| Steps A-B | 22 | 22 | 22 | 22 | 22 | 22 |

Data show the time taken to decode all query sequences in the samples (n=24,487 query sequences), as a function of database size. The database included the 12 Rs[9,5] sequences used in the experiments padded with randomly generated RS[9,5] sequences. These data show that the decoding time varies linearly with database size for local sequence alignment only and for Steps A-E. The decoding time is independent of database size for steps A-C.

FIG. 38 illustrates decoding time versus sample size for Steps A-C. These data show that for Steps A-C the decoding time varies linearly with sample size, with an average decoding efficiency of 5.81 seqs s-1. Note that Steps A-C capture 72.64% of query sequences. (see Table 5)

TABLE 5
Decoding time versus sample size for Steps A-C.

| | Sample size (1,000's query sequences) | | | | | | | |
|---|---|---|---|---|---|---|---|---|
| | 0.1 | 0.2 | 0.5 | 1 | 2 | 5 | 10 | 24 |
| Steps A-C | 54 | 103 | 234 | 412 | 694 | 1268 | 2076 | 4196 |
| (secs) | | | | | | | | |

RS[9.5]-Ham[7,4] sequence specifications used in experiments

TABLE 6
Sequence specifications of RS[9,5]-Ham[7,4]
fragments used in Series 1 experiments (9 mm
and 0.300 calibre firearms).

Tag_RS_1
Codeword:42-123-7-66-23-12-20-77-24
SEQ ID NO: 2
TTTCTGTTGGTGCTGATATTGC-[ATTCCGA-TCTCTCT-AAGGAAG-
TGCTACA-CGATTGA-AGCGCTG-AACCAAT-TCCATCA-CTCCTCT]-
GAAGATAGAGCGACAGGCAAGT
Tag_RS_2
Codeword: 94-99-91-66-41-96-34-86-119
SEQ ID NO: 3
TTTCTGTTGGTGCTGATATTGC-[GTTCCAG-GACACTA-TCAACTA-
TGCTACA-ACTGGCT-CACCACG-TACACAT-AGTACTA-GGTCAAT]-
GAAGATAGAGCGACAGGCAAGT
Tag_RS_3
Codeword: 24-9-108-29-7-84-61-53-99
SEQ ID NO: 4
TTTCTGTTGGTGCTGATATTGC-[CTCCTCT-GTATATG-CTGACTA-
AGAAGAG-AAGGAAG-TGTGTAT-GATCGAC-CGTACAT-GACACTA]-
GAAGATAGAGCGACAGGCAAGT
Tag_RS_4
Codeword: 117-21-27-96-57-86-19-78-17
SEQ ID NO: 5
TTTCTGTTGGTGCTGATATTGC-[ATGGTAT-TCGTAGT-CGTCATA-
CACCACG-GTAGCAC-AGTACTA-TCGCGAC-TTCTCCT-TAAGGCT]-
GAAGATAGAGCGACAGGCAAGT
Tag_RS_5
Codeword: 111-35-14-110-108-3-108-64-79
SEQ IDNO: 6
TTTCTGTTGGTGCTGATATTGC-[GATTAGT-CTGGTCG-TGGAACT-
TCCAGAG-CTGACTA-CTATAGT-CTGACTA-ACAACAT-TAGTTGA]-
GAAGATAGAGCGACAGGCAAGT
Tag_RS_6
Codeword: 104-71-8-38-69-20-73-40-18
SEQ ID NO: 7
TTTCTGTTGGTGCTGATATTGC-[TTGGTTA-CTAATCA-TCACAGC-
TCGGCTG-ATCAACT-AACCAAT-CATGCGT-AGCATCA-GACCAGC]-
GAAGATAGAGCGACAGGCAAGT
Tag_RS_7
Codeword: 22-36-46-3-123-109-47-81-126
SEQ ID NO: 8
TTTCTGTTGGTGCTGATATTGC-[CTAGCTG-GACTGAT-TCCGAGA-
CTATAGT-TCTCTCT-TGATTAG-AGGTCTC-TCTTCTC-GCTAACT]-
GAAGATAGAGCGACAGGCAAGT
Tag_RS_8
Codeword: 39-121-34-87-121-15-9-9-39
SEQ ID NO: 9
TTTCTGTTGGTGCTGATATTGC-[GAACTCT-CGTGTGC-TACACAT-
AGGCTCT-CGTGTGC-ACACATA-GTATATG-GTATATG-CAACTCT]-
GAAGATAGAGCGACAGGCAAGT
Tag_RS_9
Codeword: 44-81-109-8-111-36-117-74-5
SEQ ID NO: 10
TTTCTGTTGGTGCTGATATTGC-[AGCTAGT-TCTTCTC-TGATTAG-
TCACAGC-GATTAGT-GACTGAT-ATGGTAT-GTGGTGC-CTACGAC]-
GAAGATAGAGCGACAGGCAAGT
Tag_RS_10
Codeword: 71-37-4-45-15-21-30-39-114
SEQ ID NO: 11
TTTCTGTTGGTGCTGATATTGC-[CTAATCA-ACTAATC-CGACCAG-
CTGTGAT-ACACATA-TCGTACT-GGCTAAC-GAACTCT TACCATA]-
GAAGATAGAGCGACAGGCAAGT
Tag_RS_11
Codeword: 47-37-127-95-5-26-65-71-51
SEQ ID NO: 12
TTTCTGTTGGTGCTGATATTGC-[AGGTCTC-ACTAATC-TTGCAAT-
AAGAGGA-CTACGAC-TATCGCA-TCATCAT-CTAATCA-GATATCA]-
GAAGATAGAGCGACAGGCAAGT
Tag_RS_12
Codeword: 85-81-31-114-95-24-121-56-45
SEQ ID NO: 13
TTTCTGTTGGTGCTGATATTGC-[GCTGGTC-TCTTCTC-GAGGAGA-
TACCATA-AAGAGGA-CTCCTCT-CGTGTGC-ATGCACG-CTGTGAT]-
GAAGATAGAGCGACAGGCAAGT

Only the template strand for each tag is given in the 5′→3′ direction. The codeword is shown in bold in square brackets, with each Ham[7,4] symbol delimited by a ‘-’. Parity symbols are shown in grey. Universal primer site sequences that flank the codeword are in plain text.

### Key Advantages of the Disclosed Invention

The disclosed invention is a system for product tracing and verification where supply chain information is stored in physical oligonucleotide tags that are integrated into a product and backed up on an immutable blockchain. Core capabilities of the disclosed invention include full unbroken supply chain coverage, high resolution tracing (at the level of an ingredient and product unit), automatic transfer of chain information upon product mixing (no requirement to authenticate each transaction), last legitimate node traceback capabilities, protection against counterfeiting, and product authentication.

Full supply chain coverage. The use of oligonucleotide fragment as a product integrated storage media in combination with blockchain technology offers several clear advantages over previous tracing systems. Firstly, the incorporation of encoded oligonucleotide fragments into a product creates an immutable link between the physical product and data stored on a virtual blockchain. This represents a step change in security. All previous blockchain-based approaches use a package technology that only represents a proxy for whatever physical good change hands. Secondly, the property that the oligonucleotide tags are transferred automatically upon mixing means that a tag added at one node can be traced to all nodes downstream in a supply chain. Previous systems require each transaction in a supply chain to be authenticated, and are therefore more labour intensive to execute. Thirdly, the use of unique node hashes computed from the oligonucleotide tags in a product, combined with blockchain technology, permit additional information to be directly appended the tags in a product. Fourthly, because the oligonucleotide markers are incorporated into the product, traceback capabilities or chain repair can be performed on an unpackaged product (for e.g. a product altered by an end-user or consumer). Lastly, full supply chain coverage offers may advantages for certification schemes, for example ingredients that are verified as fair trade, sustainable, or kosher/halal may be traced to a certified producer from a finished product alone.

Anti-counterfeiting and security. The disclosed invention virtually eliminates the possibility of counterfeiting because it creates an unbreakable link between the ingredients in a product, the finished product, the packaging, and product data stored in a distributed immutable blockchain. This permits, for example, the detection of counterfeit products that are: (1) cut or swapped in upstream from the point of finished product packaging (2) packaged in fake packaging (3) packaged in recycled legitimate packaging, (4) exchanged into a consignment of products where legitimate products are swapped out, and (5) out of date and re-stamped with false expiry information.

High resolution tracing capability (product, not package). The disclosed invention permits product ingredient tracking to the resolution of the individual product unit (for. e.g. tablet, infant milk formula, blended cannabis products) and not just a package or consignment of packages. Current supply chain monitoring technologies require the transaction of goods to be authenticated at each node in a supply chain or else custody is lost. This is not feasible at the resolution of a product unit or packaged product, and so node authentication is performed at the consignment level which undermines system security. For example, it is not feasible to scan individual tablets or packages of pharmaceutical products in a consignment of 10,000 packages at each node in a supply chain. The disclosed technology allows supply chain information to be recovered from each unpackaged tablet if desired.

Fraudulent/leaking node identification. In cases where counterfeit or substandard products are detected, the disclosed technology provides traceback capabilities to the last legitimate node in a supply chain from the unpackaged product alone. These capabilities allow leaking or fraudulent nodes to be detected so that targeted action can be taken. For example, the point at which products are mis-used (e.g. products illegally used a precursors in illicit drugs), counterfeited by dilution (e.g. pharmaceutical products cut with cheap excipients), or sold into unauthorised markets (parallel importing) can be detected.

Recalled products. The disclosed technology permits supply chain information to be recovered from the unpackaged end-product alone. This capability permits the detection of nodes where substandard products enter a supply chain. It also offers a rapid and definitive test to dissociate a brand from substandard and/or counterfeit products.

### Examples of Practical Use Cases for the Disclosed Technology

Palm oil. Palm oil is used is a wide range of products including food products, cosmetics, cleaning products and pharmaceuticals. Palm oil production is also linked to deforestation, biodiversity loss and poor work conditions. The disclosed technology may be integrated with existing certification schemes (for e.g RSPO) so that the origin of palm oil can be traced back to a sustainably certified manufacturer from the end product alone.

Pharmaceuticals. Counterfeit pharmaceuticals are responsible for one million deaths and cost the industry $100B each year. Incidents of drug counterfeiting are increasing with the rise of online pharmacies. Additionally, in many developing and transition economies, medications are sold as unpackaged individual tablets or doses. The capacity to recover supply chain information from an individual tablet alone could address the massive human and economic cost of fake pharmaceuticals.

Cannabis products. The cosmetic and medicinal cannabis industry is highly exposed to counterfeiting from backyard and recreational growers. Fake products present serious concerns as the active compound content in cannabis (THC, CBD) may vary widely in plants that are grown under different conditions and across different plant strains. Fake medicinal products that have not be subjected to stringent quality control steps, and contain sub-therapeutic cannabinoid levels, may lack therapeutic efficacy. Additionally, in some countries such as the USA, products must be grown, manufactured, and sold within state boundaries for tax purposes. The ease with which products may cross state boundaries could result in the loss in billions of dollars in tax revenue. The disclosed invention offers a means to track material from the ‘plant to product’, as well as mark various mixing and quality control steps along the manufacturing/supply chain. This information can be recovered from the unpackaged end product alone, and thereby address the problems highlighted above.

Illicit drug precursors (e.g. methamphetamine). The disclosed technology may be used to traceback the chain of custody of products that are misused. For example, legal ingredients used as precursors for the manufacture of illicit drugs, such as methamphetamine, may be traced to the last legitimate node in a supply chain from a drug sample alone. This capability may be useful for pinpointing fraudulent or leaking nodes in a supply chain, and gathering intelligence on how narcotics networks operate.

Kosher and Halal. Kosher and Halal products cannot be identified by the end product alone (there is no test of Kosher and Halal). The disclosed technology may be used to verify and track products from certified Kosher and Halal producers, and thereby address widespread counterfeiting problems in the industry.

Milk products. Counterfeit milk products are frequently detected in Asian markets, and have resulted in the hospitalisation of more than 50,000 infants from melamine poisoning since 2008. The capacity to recover and verify all supply chain information, from the milk product alone, could address this problem.

Ammunition. Recent advances in firearms technology have exacerbated the already difficult task of detecting illicit arms and ammunition transfers. In 2012, firearms were responsible for 41% of non-conflict homicides worldwide, with approximately 57% of these incidents remaining unsolved. In 2016, President Obama and the American Medical Association declared gun violence a public health concern, which is estimated to cost the US economy $229 billion each year - even more than the cost of obesity. The advent of modular, polymer, and 3D printed guns have also brought new challenges for firearms tracing and registration. The capacity to label and trace oligonucleotide tagged ammunition to the bullet entry wound has been demonstrated previously. The innovation disclosed offers a way to trace and trace crime via labelled ammunition.

Other applications. The disclosed technology may be used to track and trace many other products including, but not limited to: wine, cosmetics, precious stones, chemicals, fertilizers, bank notes, casino chips, and luxury items.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method for verifying a product's identity, the method comprising:
generating a first oligonucleotide sequence;
calculating a first hash value of the first oligonucleotide sequence, the first hash value being associated with the product;
synthesising the first oligonucleotide sequence;
adding the synthesised oligonucleotide sequence to the product;
sequencing a second oligonucleotide sequence from the product;
calculating a second hash value of the sequenced oligonucleotide sequence; and
comparing the second hash value to the first hash value associated with the product to verify the product's identity.

2. The method of claim 1, wherein the first hash value is incorporated onto a package containing the product.

3. The method of claim 1, wherein the first hash value is stored in a block chain.

4. The method of claim 3, wherein the block chain is part of a distributed ledger.

5. The method of claim 1, wherein calculating the first hash value and the second hash value is based on additional data and the additional data comprises one or more of:
product identifier;
entity identifier:
shared secret;
padding data;
public key;
time stamp;
counter; and
product-unique product identifier.

6. (canceled)

7. The method of claim 1, further comprising generating the first oligonucleotide sequence by encoding a digital word into the first oligonucleotide sequence.

8. The method of claim 7, wherein encoding the digital word is based on an error-correcting code.

9. The method of claim 8, wherein encoding the digital word comprises:
generating Hamming code words;
mapping sets of the Hamming code words to a Galois field, and
generating a Reed Solomon (RS) code word to thereby generate a robust code word that is robust against sequencing and synthesis errors.

10. The method of claim 7 , wherein the digital word is private to an entity performing the method.

11. (canceled)

12. The method of claim 1, wherein the method further comprises amplifying the second oligonucleotide sequence by a polymerase chain reaction (PCR) using a secret set of primers which hybridise to primer sites on the second oligonucleotide sequence.

13. The method of claim 1, wherein an entity downstream in a supply chain adds a third oligonucleotide sequence to the product.

14. The method of claim 13, wherein adding the third oligonucleotide sequence to the product comprises calculating a third hash value associated with the product.

15. The method of claim 14, wherein the third hash value is calculated based on one or more upstream hash values to thereby represent an order of added oligonucleotide sequences forming a chain of hash values.

16. (canceled)

17. The method of claim 15, further comprising
sequencing the third oligonucleotide sequence;
calculating a fourth hash value for each of multiple combinations of the second hash value and the fourth hash value; and
comparing the fourth hash value for each of the multiple combinations to the third hash value to identify the product's identity where one of the multiple combinations provides a match.

18. The method of claim 17, wherein the method comprises:
identifying an upstream node for which the fourth hash value for one of the multiple combinations matches; and
calculating hash values only for combinations that relate to nodes downstream from the identified upstream node.

19. The method of claim 18, wherein adding the third oligonucleotide sequence to the product comprises facilitating ligation of the third oligonucleotide sequence to the first oligonucleotide sequence.

20. The method of claim 19, wherein the third oligonucleotide sequence added by the entity downstream in the supply chain is indicative of a position of the entity within the supply chain.

21. (canceled)

22. The method of claim 1, wherein calculating the second hash value comprises decoding the sequenced oligonucleotide sequence in one direction and upon unsuccessful decoding, decoding the sequenced oligonucleotide sequence in an opposite direction.

23. The method of claim 1, further comprising aligning the sequenced second oligonucleotide sequence against a stored oligonucleotide sequence, wherein calculating the second hash value is based on the aligned nucleotide sequence.

24. The method of claim 23, wherein generating the first oligonucleotide sequence is based on multiple code symbols and the method comprises aligning the sequenced second oligonucleotide sequence against the multiple code symbols.

25. The method of claim 23, wherein generating the first oligonucleotide sequence comprises generating multiple codewords and the method comprises aligning the sequenced second oligonucleotide sequence against previously decoded codewords or a database of codewords.

26. The method of claim 25, wherein the method further comprises determining a sequencing error and selectively, based on the sequencing error, performing alignment against multiple code symbols or against multiple codewords.

27. A method for manufacturing an identifiable product, the method comprising:
manufacturing the product;
generating a first oligonucleotide sequence;
calculating a first hash value of the first oligonucleotide sequence, the first hash value being associated with the product;
synthesising the first oligonucleotide sequence; and
adding the synthesised oligonucleotide sequence to the product to allow sequencing and comparing a second hash value of the sequencing result to the first hash value to verify the product's identity.

28. A method of verifying a product's identity, the method comprising:
a) providing a product to which a first oligonucleotide has been added,
b) obtaining the sequence of the first oligonucleotide and calculating a hash value from the sequence, and
c) comparing the hash value to a predetermined value for the product to verify the product's identity.

29. Software that, when executed by a computer, causes the computer to perform the method of claim 1.

30. An identifiable product comprising:
one or more product constituents; and
a synthesised oligonucleotide sequence added to the one or more product constituents, wherein the synthesised oligonucleotide sequence is associated with a first hash value to allow comparing a second hash value of a result from sequencing the synthesised oligonucleotide sequence to the first hash value to verify the product's identity.

31. The product of claim 30, further comprising a package containing the product, wherein the first hash value is incorporated onto the package.
